(19) United States
(12) Patent Application Publication
(10) Pub. No.: US 2023/0384677 A1
(43) Pub. Date: Nov. 30, 2023

(54) ONIUM SALT COMPOUND, POLYMER, RESIST COMPOSITION, AND PATTERNING PROCESS

(71) Applicant: Shin-Etsu Chemical Co., Ltd., Tokyo (JP)

(72) Inventors: Tomomi Watanabe, Joetsu-shi (JP); Takayuki Fujiwara, Joetsu-shi (JP); Tomonari Noguchi, Joetsu-shi (JP)

(73) Assignee: Shin-Etsu Chemical Co., Ltd., Tokyo (JP)

(21) Appl. No.: 18/197,234

(22) Filed: May 15, 2023

(30) Foreign Application Priority Data
May 24, 2022 (JP) 2022-084705

(51) Int. Cl. G03F 7/039; C08F 220/22; C08F 212/14; C08F 220/28; G03F 7/038; C07C 309/12

(52) U.S. Cl. CPC G03F 7/0397; C08F 220/22; C08F 212/24; C08F 220/281; G03F 7/0382; C07C 309/12; C08F 2800/10

(57) ABSTRACT

An onium salt compound consisting of a sulfonate anion having the structure that a polymerizable unsaturated bond is linked to an iodized aromatic group via a carbon chain of two or more carbon atoms and a sulfonium or iodonium cation is provided. A resist composition comprising a polymer comprising repeat units derived from the onium salt has a high sensitivity and forms a pattern with improved LWR or CDU, independent of whether it is of positive or negative tone.

### CROSS-REFERENCE TO RELATED APPLICATIONS

This non-provisional application claims priority under 35 U.S.C. § 119(a) on Patent Application No. 2022-084705 filed in Japan on May 24, 2022, the entire contents of which are hereby incorporated by reference.

### TECHNICAL FIELD

This invention relates to an onium salt compound, a polymer, a resist composition, and a pattern forming process.

### BACKGROUND ART

To meet the demand for higher integration density and operating speed of LSIs, the effort to reduce the pattern rule is in rapid progress. Logic devices are manufactured in a large scale using a multi-patterning lithography process based on ArF lithography. To form patterns of smaller size, studies are made on resist compositions adapted for lithography of shorter wavelength, typically EB or EUV lithography. As the miniaturization technology proceeds toward smaller pattern feature size, it is regarded more important to improve lithography properties including pattern profile, contrast, line width roughness (LWR) of line patterns, and critical dimension uniformity (CDU) of hole patterns.

With the rapid progress of reducing the pattern feature size, LWR and CDU become more noticeable. It is pointed out that lithography properties are largely affected by the segregation and agglomeration of a base polymer and a photoacid generator and acid diffusion. There is a propensity that LWR is degraded as the resist film becomes thinner. The degradation of LWR caused by resist film thinning to comply with further miniaturization becomes a serious problem.

For the EUV resist composition, it is necessary to achieve a high sensitivity, high resolution and low LWR at the same time. As the acid diffusion distance is shortened, the outcome is a smaller LWR, but a lower sensitivity. For example, at a lower PEB temperature, LWR becomes smaller, but sensitivity becomes lower. Also, when the amount of a quencher added is increased, LWR becomes smaller, but sensitivity becomes lower. It is necessary to overcome the tradeoff relationship between sensitivity and LWR.

For suppressing acid diffusion, various attempts have been made to introduce a bulky substituent or polar group into a photoacid generator. Patent Document 1 describes a photoacid generator having 2-acyloxy-1,1,3,3,3-pentafluoropropane-1-sulfonic acid which has solubility in resist solvent and stability and allows for broad molecular design. In particular, a photoacid generator having 2-(1-adamantyloxy)-1,1,3,3,3-pentafluoropropane-1-sulfonic acid, i.e., having a bulky substituent introduced therein, shows reduced acid diffusion. Resist compositions comprising such photoacid generators are still insufficient in high-level control of acid diffusion, and fail to meet the lithography performance when lithography properties such as mask error factor (MEF), pattern profile, and sensitivity are comprehensively considered.

Patent Document 2 discloses a polymer containing repeat units derived from a sulfonic acid onium salt having a polymerizable unsaturated bond, i.e., so-called polymer-bound photoacid generator. Patent Document 3 describes a polymer obtained from polymerization of an acryloyloxyphenyldiphenylsulfonium salt. For the purpose of improving the LWR of a polyhydroxystyrene resin-based resist composition, Patent Document 4 describes to introduce the acryloyloxyphenyldiphenylsulfonium salt into the base polymer via polymerization. Since the acid generator is bound to the polymer at its cation side, the sulfonic acid generated by exposure to high-energy radiation remains unchanged from the sulfonic acid generated from the conventional photoacid generator, and is insufficient to suppress acid diffusion. Patent Documents 5 to 7 describe resist compositions comprising a polymer obtained from polymerization of a sulfonium salt and having a fluorinated skeleton. These resist compositions are successful in improving LWR to some extent. Since the sulfonic acid which is bound to the polymer is released upon exposure, acid diffusion is quite short. A higher sensitivity can be achieved by increasing the proportion of the acid generator. With the aim to ameliorate the tradeoff relationship between sensitivity and LWR. Patent Document 7 refers to a polymer-bound photoacid generator having the structure that an anion skeleton having a polymerizable unsaturated bond contains fluorine. By introducing iodine which is highly absorptive to EUV radiation, a higher sensitivity is achieved. However, the degree of freedom of the sulfonic acid in the polymer is low due to the robust molecular structure, leaving the problems of localized distribution and poor solvent solubility. Despite low acid diffusion, lithography properties including LWR are left unsatisfactory as viewed from the aspect of forming small size patterns.

For the purposes of taking advantage of the short wavelength of the energy source and improving the lithography properties, it is quite important that the polymer-bound photoacid generator is optimized in structure so as to achieve a good balance of sensitivity, LWR and CDU.

### CITATION LIST

Patent Document 1: JP-A 2007-145797 (U.S. Pat. No. 7,511,169)
Patent Document 2: JP 4425776
Patent Document 3: JP-A H04-230645
Patent Document 4: JP-A 2005-084365
Patent Document 5: JP-A 2010-116550
Patent Document 6: JP-A 2010-077404
Patent Document 7: JP 6973274

### SUMMARY OF THE INVENTION

It is desired to develop a resist composition exhibiting a higher sensitivity and capable of reducing the LWR of line patterns or improving the CDU of hole patterns.

An object of the invention is to provide a resist composition which achieves a high sensitivity, minimal LWR and improved CDU independent of whether it is of positive or negative tone, and a pattern forming process using the resist composition.

The inventors have found that a resist composition having a high sensitivity, improved LWR or CDU, high contrast, high resolution and wide process margin is obtained from a polymer comprising repeat units derived from an onium salt compound consisting of a sulfonate anion having the structure that a polymerizable unsaturated bond is linked to an aromatic group substituted with at least one iodine atom via a carbon chain having at least two carbon atoms and a sulfonium or iodonium cation.

In one aspect, the invention provides an onium salt compound consisting of a sulfonate anion having the structure that a polymerizable unsaturated bond is linked to an aromatic group substituted with at least one iodine atom via a carbon chain having at least two carbon atoms and a sulfonium or iodonium cation.

The onium salt compound preferably has the formula (1).

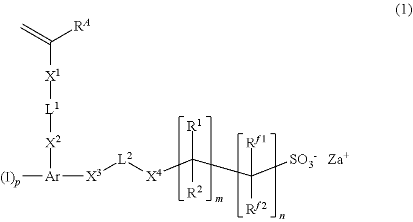

Herein m is an integer of 0 to 4, n is an integer of 1 to 4, p is an integer of 1 to 4,
R^{A} is hydrogen or methyl,
R¹ and R² are each independently hydrogen, fluorine, or a C₁-C₁₀ hydrocarbyl group which may contain a heteroatom. R¹ and R² may bond together to form a ring with the carbon atom to which they are attached,
R^{f1} and R^{f2} are each independently hydrogen, fluorine or trifluoromethyl, at least one of R^{f1} and R^{f2} is fluorine or trifluoromethyl,
X¹ to X⁴ are a single bond, ether bond, ester bond, sulfonic ester bond or carbonate bond,
L¹ is a C₂-C₁₅ hydrocarbylene group in which some or all hydrogen may be substituted by a heteroatom-containing moiety, and some constituent —CH₂— may be replaced by an ether bond, ester bond or lactone ring-containing moiety,
L² is a single bond or a C₁-C₁₅ hydrocarbylene group in which some or all hydrogen may be substituted by a heteroatom-containing moiety, and some constituent —CH₂— may be replaced by an ether bond, ester bond or lactone ring-containing moiety,
Ar is a C₆-C₁₅ (p+2)-valent aromatic group in which some or all hydrogen may be substituted by a substituent, and
Za⁺ is a sulfonium or iodonium cation.

More preferably, the anion has the formula (1a).

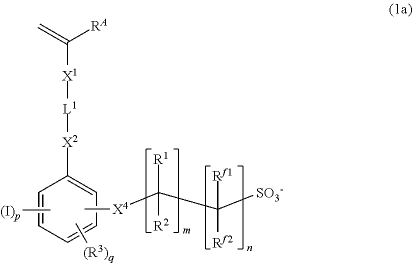

Herein m, n, p, R^{A}, R¹, R², R^{f1}, R^{f2}, X¹, X², X⁴ and L¹ are as defined above,
q is an integer of 0 to 3, q+p is from 1 to 4,
R³ is a hydroxy group, fluorine, amino group, sulfo group, or a C₁-C₁₅ hydrocarbyl group in which some or all hydrogen may be substituted by a heteroatom-containing moiety, and some constituent —CH₂— may be replaced by —O—, —C(═O)— or —N(R^{N})—, R^{N} is hydrogen or a C₁-C₁₀ hydrocarbyl group in which some or all hydrogen may be substituted by a heteroatom-containing moiety, and some constituent —CH₂— may be replaced by —O—, —C(═O)— or —S(═O)₂—.

Even more preferably, the anion has the formula (1b):

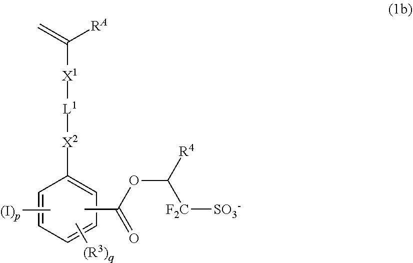

wherein p, q, R^{A}, R³, X¹, X², and L¹ are as defined above, and R is hydrogen or trifluoromethyl.

In a preferred embodiment, Za⁺ is a cation having the formula (Z-1) or (Z-2).

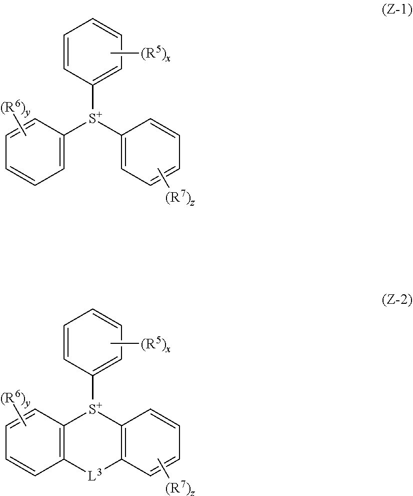

Herein R⁵, R⁶, and R⁷ are each independently halogen, hydroxy or a C₁-C₁₅ hydrocarbyl group in which some or all hydrogen may be substituted by a heteroatom-containing moiety, and some constituent —CH₂— may be replaced by —O—, —C(═O)—, —S—, —S(═O)—, —S(═O)₂— or —N(R^{N})—,
L³ is a single bond, —CH₂—, —O—, —C(═O)—, —S—, —S(═O)—, —S(═O)₂— or —N(R^{N})—,
R^{N} is hydrogen or a C₁-C₁₀ hydrocarbyl group in which some or all hydrogen may be substituted by a heteroatom-containing moiety, and some constituent —CH₂— may be replaced by —O—, —C(═O)— or —S(═O)₂—,
x, y and z are each independently an integer of 0 to 5, with the proviso that when x is 2 or more, a plurality of R⁵ may be identical or different, and two R⁵ may bond together to form a ring with the carbon atoms on the benzene ring to which they are attached, when y is 2 or more, a plurality of R⁶ may be identical or different, and two R⁶ may bond together to form a ring with the carbon atoms on the benzene ring to which they are attached, when z is 2 or more, a plurality of R⁷ may be identical or different, and two R⁷ may bond together to form a ring with the carbon atoms on the benzene ring to which they are attached.

In another aspect, the invention provides a polymer comprising repeat units derived from the onium salt compound defined herein.

In a further aspect, the invention provides a resist composition comprising a base polymer containing the polymer defined herein and an organic solvent.

In a preferred embodiment, the polymer further comprises repeat units having the formula (b1) or (b2).

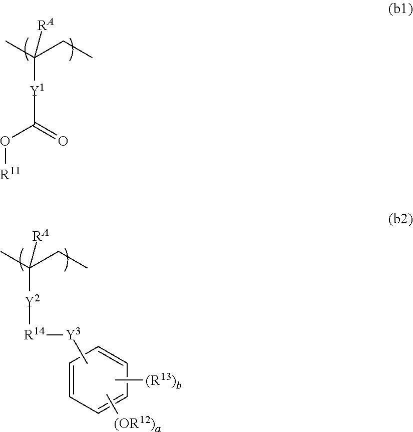

Herein R^{A} is as defined above,
Y¹ is a single bond, phenylene, naphthylene, or a C₁-C₁₂ linking group containing at least one moiety selected from ester bond and lactone ring,
Y² is a single bond or ester bond,
Y³ is a single bond, ether bond or ester bond,
R¹¹ and R¹² are each independently an acid labile group,
R¹³ is fluorine, trifluoromethyl, cyano or a C₁-C₆ saturated hydrocarbyl group,
R¹⁴ is a single bond or a C₁-C₆ alkanediyl group in which some —CH₂— may be replaced by an ether bond or ester bond, and
a is 1 or 2, b is an integer of 0 to 4, and a+b is from 1 to 5.

In a preferred embodiment, the polymer further comprises repeat units having the formula (c).

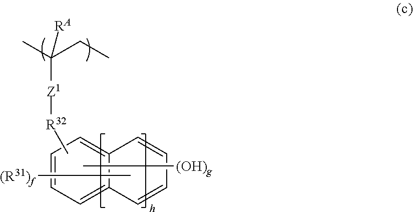

Herein R^{A} is as defined above,
Z¹ is a single bond, ether bond, ester bond, sulfonic ester bond, or carbonate bond,
R³¹ is fluorine, iodine or a C₁-C₁₀ hydrocarbyl group in which some —CH₂— may be replaced by —O— or —C(═O)—,
R³² is a single bond or a C₁-C₁₅ hydrocarbylene group,
f is an integer meeting 0≤f≤5+2h−g, g is an integer of 1 to 3, and h is an integer of 0 to 2.

The resist composition may further comprise a quencher, a photoacid generator, and/or a surfactant.

In a further aspect, the invention provides a pattern forming process comprising the steps of applying the resist composition defined herein onto a substrate to form a resist film thereon, exposing the resist film to high-energy radiation, and developing the exposed resist film in a developer.

Typically, the high-energy radiation is ArF excimer laser of wavelength 193 nm, KrF excimer laser of wavelength 248 nm. EB, or EUV of wavelength 3 to 15 nm.

### Advantageous Effects of Invention

A resist composition comprising a base polymer containing a polymer having the specific onium salt structure has many advantages including reduced acid diffusion, high sensitivity, high resolution, a good balance of lithography properties, and high compatibility, and forms a resist pattern with a minimal number of defects.

### BRIEF DESCRIPTION OF THE DRAWINGS

FIG. 1 is a diagram showing the ¹H-NMR spectrum of Compound C-2 obtained in Example 1-1.

FIG. 2 is a diagram showing the 1H-NMR spectrum of Compound C-3 obtained in Example 1-1.

FIG. 3 is a diagram showing the ¹H-NMR spectrum of Compound C-4 obtained in Example 1-1.

FIG. 4 is a diagram showing the ¹H-NMR spectrum of Compound C-7 obtained in Example 1-1.

FIG. 5 is a diagram showing the ¹⁹F-NMR spectrum of Compound C-7 obtained in Example 1-1.

FIG. 6 is a diagram showing the ¹H-NMR spectrum of onium salt compound PAG-1 obtained in Example 1-1.

FIG. 7 is a diagram showing the ¹⁹F-NMR spectrum of onium salt compound PAG-1 obtained in Example 1-1.

FIG. 8 is a diagram showing the ¹H-NMR spectrum of Compound C-10 obtained in Example 1-2.

FIG. 9 is a diagram showing the ¹H-NMR spectrum of Compound C-Il obtained in Example 1-2.

FIG. 10 is a diagram showing the ¹H-NMR spectrum of Compound C-12 obtained in Example 1-2.

FIG. 11 is a diagram showing the ¹H-NMR spectrum of Compound C-14 obtained in Example 1-2.

FIG. 12 is a diagram showing the ¹⁹F-NMR spectrum of Compound C-14 obtained in Example 1-2.

FIG. 13 is a diagram showing the ¹H-NMR spectrum of onium salt compound PAG-4 obtained in Example 1-2.

FIG. 14 is a diagram showing the ¹⁹F-NMR spectrum of onium salt compound PAG-4 obtained in Example 1-2.

FIG. 15 is a diagram showing the ¹H-NMR spectrum of Compound C-16 obtained in Example 1-3.

FIG. 16 is a diagram showing the ¹H-NMR spectrum of Compound C-17 obtained in Example 1-3.

FIG. 17 is a diagram showing the ¹H-NMR spectrum of Compound C-18 obtained in Example 1-3.

FIG. 18 is a diagram showing the ¹H-NMR spectrum of Compound C-19 obtained in Example 1-3.

FIG. 19 is a diagram showing the ¹H-NMR spectrum of Compound C-20 obtained in Example 1-3.

FIG. 20 is a diagram showing the ¹⁹F-NMR spectrum of Compound C-20 obtained in Example 1-3.

FIG. 21 is a diagram showing the ¹H-NMR spectrum of onium salt compound PAG-7 obtained in Example 1-3.

FIG. 22 is a diagram showing the ¹⁹F-NMR spectrum of onium salt compound PAG-7 obtained in Example 1-3.

FIG. 23 is a diagram showing the ¹H-NMR spectrum of onium salt compound PAG-2 obtained in Example 1-4.

FIG. 24 is a diagram showing the ¹⁹F-NMR spectrum of onium salt compound PAG-2 obtained in Example 1-4.

### DETAILED DESCRIPTION OF THE INVENTION

The singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. The terms “group” and “moiety” are interchangeable. As used herein, the term “fluorinated” or “iodized” compound means a fluorine or iodine-containing compound. In chemical formulae, the broken line denotes a valence bond, Me stands for methyl, and Ac for acetyl.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
CDU: critical dimension uniformity

Onium Salt

One embodiment of the invention is an onium salt compound consisting of a sulfonate anion having the structure that a polymerizable unsaturated bond is linked to an aromatic group substituted with at least one iodine atom via a carbon chain having at least two carbon atoms and a sulfonium or iodonium cation.

The onium salt compound preferably has the formula (1).

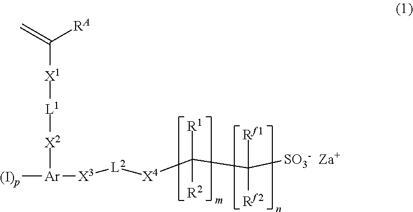

In formula (1), m is an integer of 0 to 4, n is an integer of 1 to 4, and p is an integer of 1 to 4. Preferably, m is an integer of 0 to 2, n is 1 or 2, and p is an integer of 1 to 3.

In formula (1), R^{A} is hydrogen or methyl.

In formula (1), R¹ and R² are each independently hydrogen, fluorine, or a C₁-C₁₀ hydrocarbyl group which may contain a heteroatom. R¹ and R² may bond together to form a ring with the carbon atom to which they are attached. The hydrocarbyl group may be saturated or unsaturated and straight, branched or cyclic. Some or all of the hydrogen atoms in the hydrocarbyl group may be substituted by a moiety containing a heteroatom such as oxygen, sulfur, nitrogen or halogen, and some constituent —CH₂— in the hydrocarbyl group may be replaced by a moiety containing a heteroatom such as oxygen, sulfur or nitrogen, so that the group may contain fluorine, chlorine, bromine, iodine, hydroxy, cyano, carbonyl, ether bond, ester bond, sulfonic ester bond, carbonate bond, lactone ring, sultone ring, carboxylic anhydride (—C(═O)—O—C(═O)—) or haloalkyl moiety.

Examples of the optionally heteroatom-containing hydrocarbyl group represented by R¹ and R² include C₁-C₁₀ alkyl groups such as methyl, trifluoromethyl, ethyl, n-propyl, isopropyl, n-butyl, isobutyl, sec-butyl, tert-butyl, n-pentyl, n-hexyl, n-octyl, 2-ethylhexyl, n-nonyl, and n-decyl; C₃-C₁₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, and adamantyl: C₂-C₁₀ alkenyl groups such as vinyl, allyl, propenyl, butenyl and hexenyl; C₃-C₁₀ cyclic unsaturated aliphatic hydrocarbyl groups such as cyclohexenyl; C₆-C₁₀ aryl groups such as phenyl, 2-hydroxyphenyl, 4-hydroxyphenyl, 2-methoxyphenyl, 3-methoxyphenyl, 4-ethoxyphenyl, 4-fluorophenyl, 4-iodophenyl, 4-n-butylphenyl, 4-tert-butylphenyl, 4-trifluoromethylphenyl, 2,4-dimethylphenyl, 2,4,6-trimethylphenyl, and naphthyl; C₇-C₁₀ aralkyl groups such as benzyl, 1-phenylethyl and 2-phenylethyl; C₆-C₁₀ heteroaryl groups such as thienyl; and combinations thereof. Inter alia, hydrogen, fluorine and trifluoromethyl are preferred.

In formula (1), R^{f1} and R^{f2} are each independently hydrogen, fluorine or trifluoromethyl, at least one of R^{f1} and R^{f2} is fluorine or trifluoromethyl. Preferably both R^{f1} and R^{f2} bonded to the α-carbon of —SO₃⁻ group are fluorine.

In formula (1), X¹ to X⁴ are each independently a single bond, ether bond, ester bond, sulfonic ester bond or carbonate bond, preferably a single bond or ester bond.

In formula (1). L¹ is a C₂-C₁₅ hydrocarbylene group. The hydrocarbylene group may be saturated or unsaturated and straight, branched or cyclic. Examples thereof include ethylene, 1,2-propanediyl, 1,3-propanediyl, 1,2-butanediyl, 2,3-butanediyl, 1,4-butanediyl, 2,3-dimethyl-2,3-butanediyl, 1,5-pentanediyl, 1,6-hexanediyl, 2,5-hexanediyl, 1,7-heptanediyl, 1,8-octanediyl, 1,9-nonanediyl, 1,10-dacanediyl, 1,3-cyclopentanediyl, 1,2-cyclohexanediyl, 1,3-cyclohexanediyl, 1,4-cyclohexanediyl, 4,6-dimethyl-1,3-cyclohexanediyl, 1,2-cyclohexanedimethylene, 1,3-cyclohexanedimethylene, 1,4-cyclohexanedimethylene, 1-ethyl-1,4-cyclohexanedimethylene, 2-cyclohexyl-1,3-propanediyl, 1,4-cyclooctanediyl, 1,5-cyclooctanediyl, 1,2-phenylene, 4-methyl-1,2-phenylene, 1,3-phenylene, 2-methyl-1,3-phenylene, 4-methyl-1,3-phenylene, 1,4-phenylene, 2-methyl-1,4-phenylene, 2-tert-butyl-1,4-phenylene, 2,3-dimethyl-1,4-phenylene, trimethyl-1,4-phenylene, 4-(methylene)phenyl, 1,2-benzenedimethylene, 1,3-benzenedimethylene, 1,4-benzenedimethylene, 1,2-naphthylene, 1,3-naphthylene, 1,4-naphthylene, 1,5-naphthylene, 1,6-naphthylene, 1,7-naphthylene, 2,3-naphthylene, 2,6-naphthylene, 2,7-naphthylene, 3,6-naphthylene, and 1,8-naphthalenedimethylene. Of these, preference is given to ethylene, 1,2-propanediyl, 1,3-propanediyl, 1,2-butanediyl, 2,3-butanediyl and 1,4-butanediyl. In the hydrocarbylene groups, some or all of the hydrogen atoms may be substituted by a moiety containing a heteroatom such as oxygen, sulfur, nitrogen or halogen, and some constituent —CH₂— may be replaced by an ether bond, ester bond or lactone ring-containing moiety.

In formula (1), L² is a single bond or a C₁-C₁₅ hydrocarbylene group. The hydrocarbylene group may be saturated or unsaturated and straight, branched or cyclic. Examples thereof include methylene, ethylene, 1,2-propanediyl, 1,3-propanediyl, 1,2-butanediyl, 2,3-butanediyl, 1,4-butanediyl, 2,3-dimethyl-2,3-butanediyl, 1,5-pentanediyl, 1,6-hexanediyl, 2,5-hexanediyl, 1,7-heptanediyl, 1,8-octanediyl, 1,9-nonanediyl, 1,10-dacanediyl, 1,3-cyclopentanediyl, 1,2-cyclohexanediyl, 1,3-cyclohexanediyl, 1,4-cyclohexanediyl, 4,6-dimethyl-1,3-cyclohexanediyl, 1,2-cyclohexanedimethylene, 1,3-cyclohexanedimethylene, 1,4-cyclohexanedimethylene, 1-ethyl-1,4-cyclohexanedimethylene, 2-cyclohexyl-1,3-propanediyl, 1,4-cyclooctanediyl, 1,5-cyclooctanediyl, 1,2-phenylene, 4-methyl-1,2-phenylene, 1,3-phenylene, 2-methyl-1,3-phenylene, 4-methyl-1,3-phenylene, 1,4-phenylene, 2-methyl-1,4-phenylene, 2-tert-butyl-1,4-phenylene, 2,3-dimethyl-1,4-phenylene, trimethyl-1,4-phenylene, 4-(methylene)phenyl, 1,2-benzenedimethylene, 1,3-benzenedimethylene, 1,4-benzenedimethylene, 1,2-naphthylene, 1,3-naphthylene, 1,4-naphthylene, 1,5-naphthylene, 1,6-naphthylene, 1,7-naphthylene, 2,3-naphthylene, 2,6-naphthylene, 2,7-naphthylene, 3,6-naphthylene, and 1,8-naphthalenedimethylene. Of these, preference is given to a single bond, methylene, ethylene, 1,2-propanediyl, and 1,3-propanediyl. In the hydrocarbylene groups, some or all of the hydrogen atoms may be substituted by a moiety containing a heteroatom such as oxygen, sulfur, nitrogen or halogen, and some constituent —CH₂— may be replaced by an ether bond, ester bond or lactone ring-containing moiety.

In formula (1), Ar is a C₆-C₁₅ (p+2)-valent aromatic group. The (p+2)-valent aromatic group is obtained by eliminating (p+2) number of hydrogen atoms from an aromatic hydrocarbon. In the aromatic group, some or all of the hydrogen atoms may be substituted by a substituent. Suitable substituents include hydroxy, fluorine, and C₁-C₁₅ hydrocarbyl groups. In the hydrocarbyl group, some or all of the hydrogen atoms may be substituted by a moiety containing a heteroatom such as oxygen, sulfur, nitrogen or halogen, and some constituent —CH₂— may be replaced by —O—, —C(═O)— or —N(R^{N})—. R^{N} is hydrogen or a C₁-C₁₀ hydrocarbyl group in which some or all of the hydrogen atoms may be substituted by a moiety containing a heteroatom such as oxygen, sulfur, nitrogen or halogen, and some constituent —CH₂— may be replaced by —O—, —C(═O)— or —S(═O)₂—. Ar is preferably an optionally substituted C₆-C₁₀ (p+2)-valent aromatic group.

Preferably, the anion in the onium salt compound having formula (1) has the formula (1a).

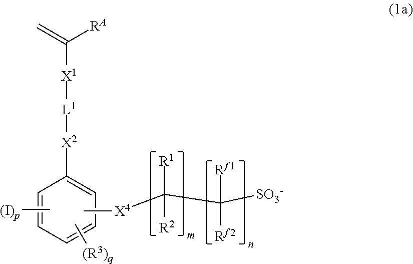

Herein m, n, p, R^{A}, R¹, R², R^{f1}, R^{f2}, X¹, X², X⁴ and L¹ are as defined above.

In formula (1a), q is an integer of 0 to 3, and q+p is from 1 to 4.

In formula (1a), R³ is a hydroxy group, fluorine, amino group, sulfo group, or a C₁-C₁₅ hydrocarbyl group in which some or all hydrogen may be substituted a moiety containing a heteroatom such as oxygen, sulfur, nitrogen or halogen, and some constituent —CH₂— may be replaced by —O—, —C(═O)— or —N(R^{N})—. Notably, the constituent —CH₂— in the hydrocarbyl group may be one bonded to a carbon atom on the benzene ring in the formula. R^{N} is hydrogen or a C₁-C₁₀ hydrocarbyl group in which some or all of the hydrogen atoms may be substituted by a moiety containing a heteroatom such as oxygen, sulfur, nitrogen or halogen, and some constituent —CH₂— may be replaced by —O—, —C(═O)— or —S(═O)₂—. When q is 2 or more, a plurality of R³ may be identical or different and two R³ may bond together to form a ring with the carbon atoms on the benzene ring to which they are attached.

The hydrocarbyl group R³ may be saturated or unsaturated and straight, branched or cyclic. Examples thereof include C₁-C₁₅ alkyl groups such as methyl, ethyl, n-propyl, isopropyl, n-butyl, isobutyl, sec-butyl, tert-butyl, n-pentyl, n-hexyl, n-octyl, 2-ethylhexyl, n-nonyl and n-decyl; C₃-C₁₅ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}-]decanyl, and adamantyl: C₂-C₁₅ alkenyl groups such as vinyl, allyl, propenyl, butenyl and hexenyl; C₃-C₁₅ cyclic unsaturated aliphatic hydrocarbyl groups such as cyclohexenyl; C₆-C₁₅ aryl groups such as phenyl, 2-hydroxyphenyl, 4-hydroxyphenyl, 2-methoxyphenyl, 3-methoxyphenyl, 4-methoxyphenyl, 4-fluorophenyl, 4-iodophenyl, 4-n-butylphenyl, 4-tert-butylphenyl, 4-tert-butoxyphenyl, 4-trifluoromethylphenyl, 2,4-dimethylphenyl, 2,4,6-trimethylphenyl, 2,4,6-triisopropylphenyl, and naphthyl; C₇-C₁₅ aralkyl groups such as benzyl, 1-phenylethyl, and 2-phenylethyl; and combinations thereof. R³ is preferably hydroxy or methyl.

Preferred examples of R³ are shown below, but not limited thereto.

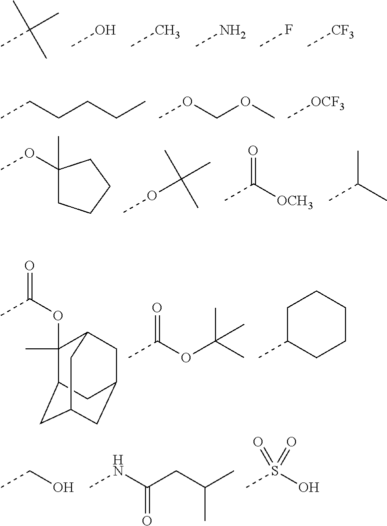

More preferably, the anion in the onium salt compound having formula (1) has the formula (1b).

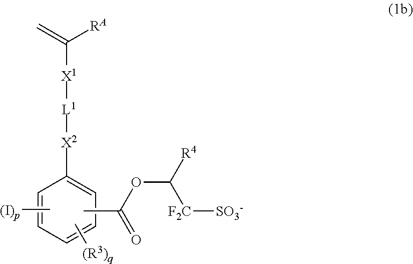

Herein p, q, R^{A}, R³, X¹, X², and L¹ are as defined above.

In formula (1b), R⁴ is hydrogen or trifluoromethyl, preferably trifluoromethyl.

Examples of the anion in the onium salt compound having formula (1) are shown below, but not limited thereto. Herein, R^{A}, R³, p and q are as defined above.

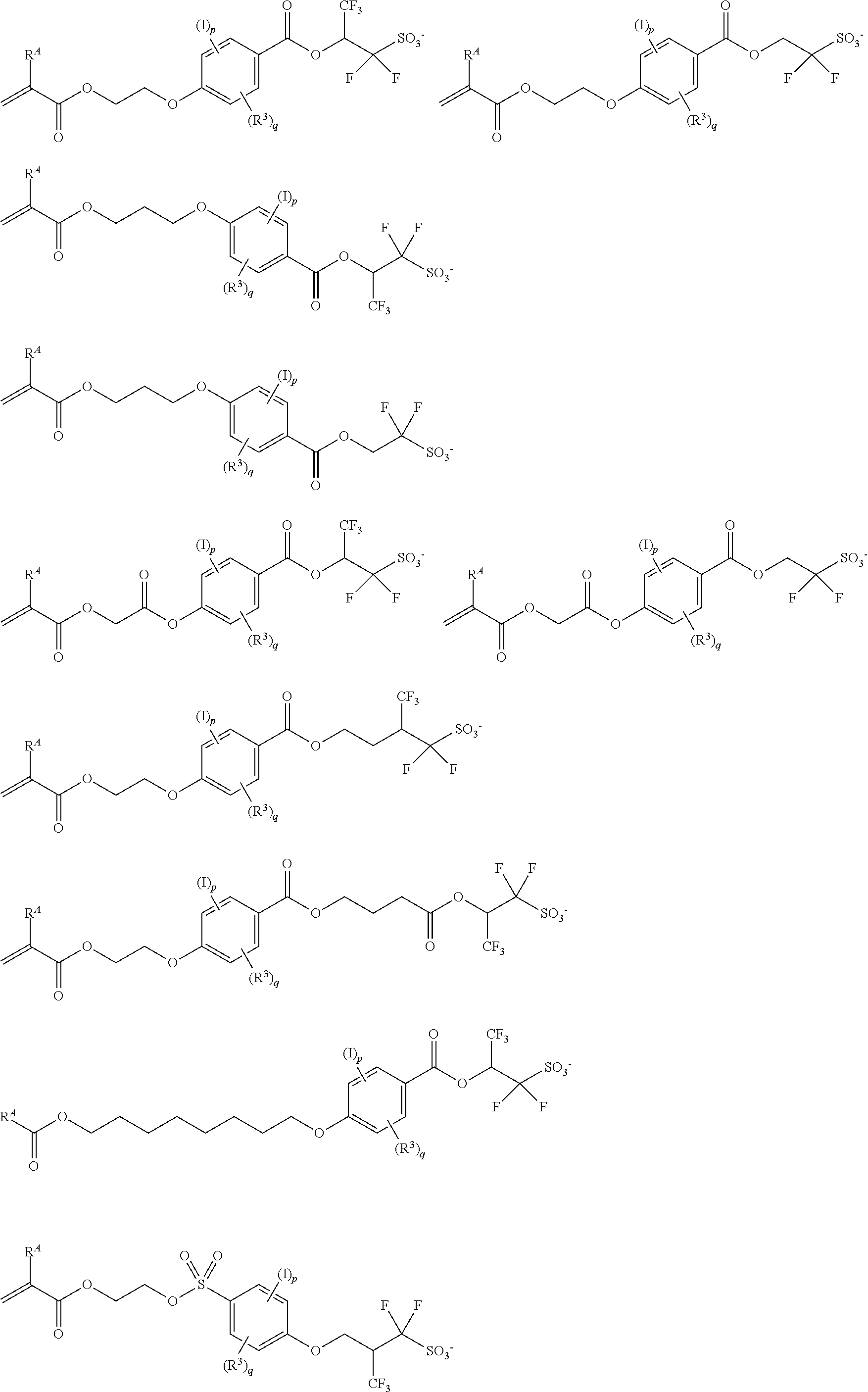

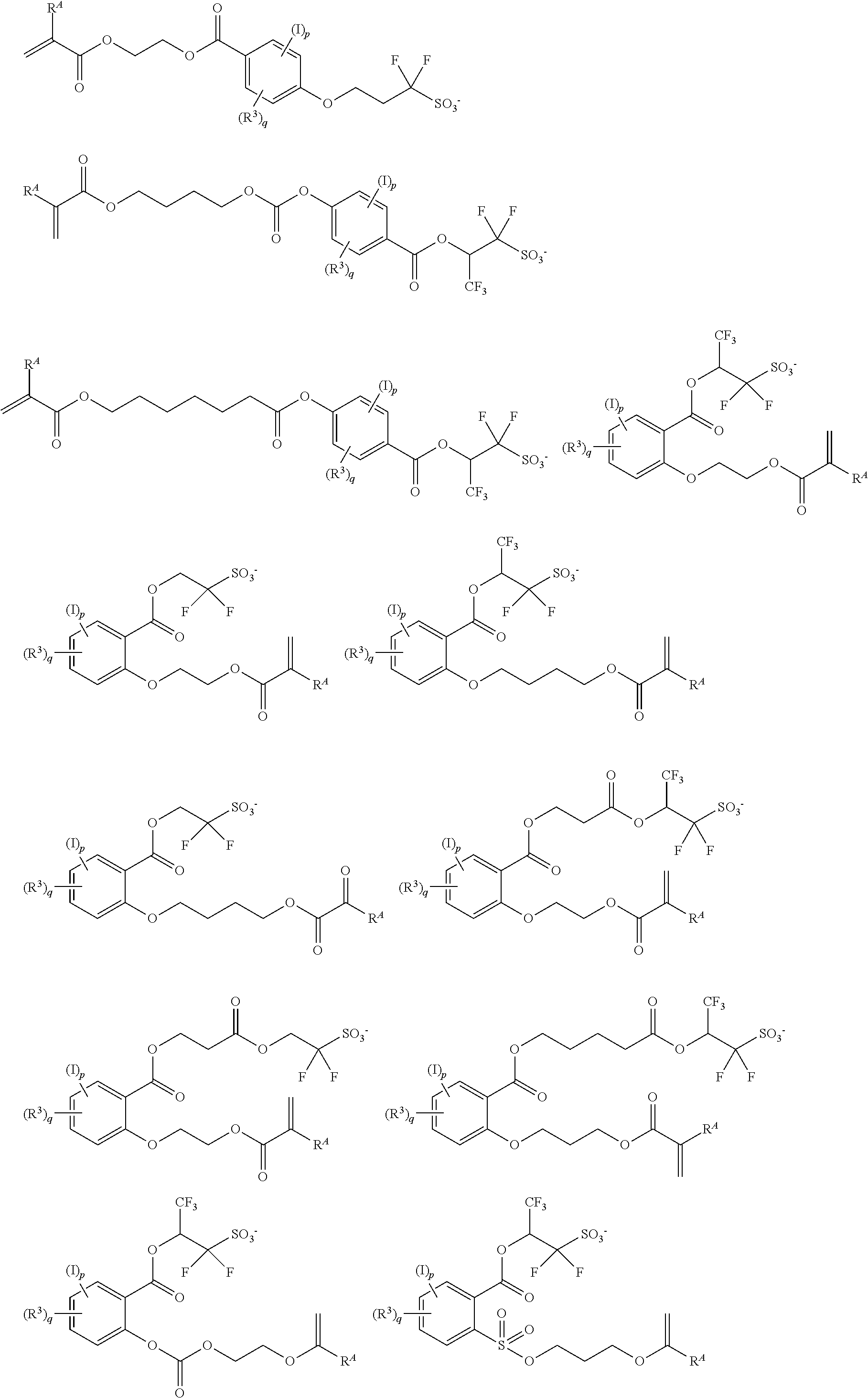

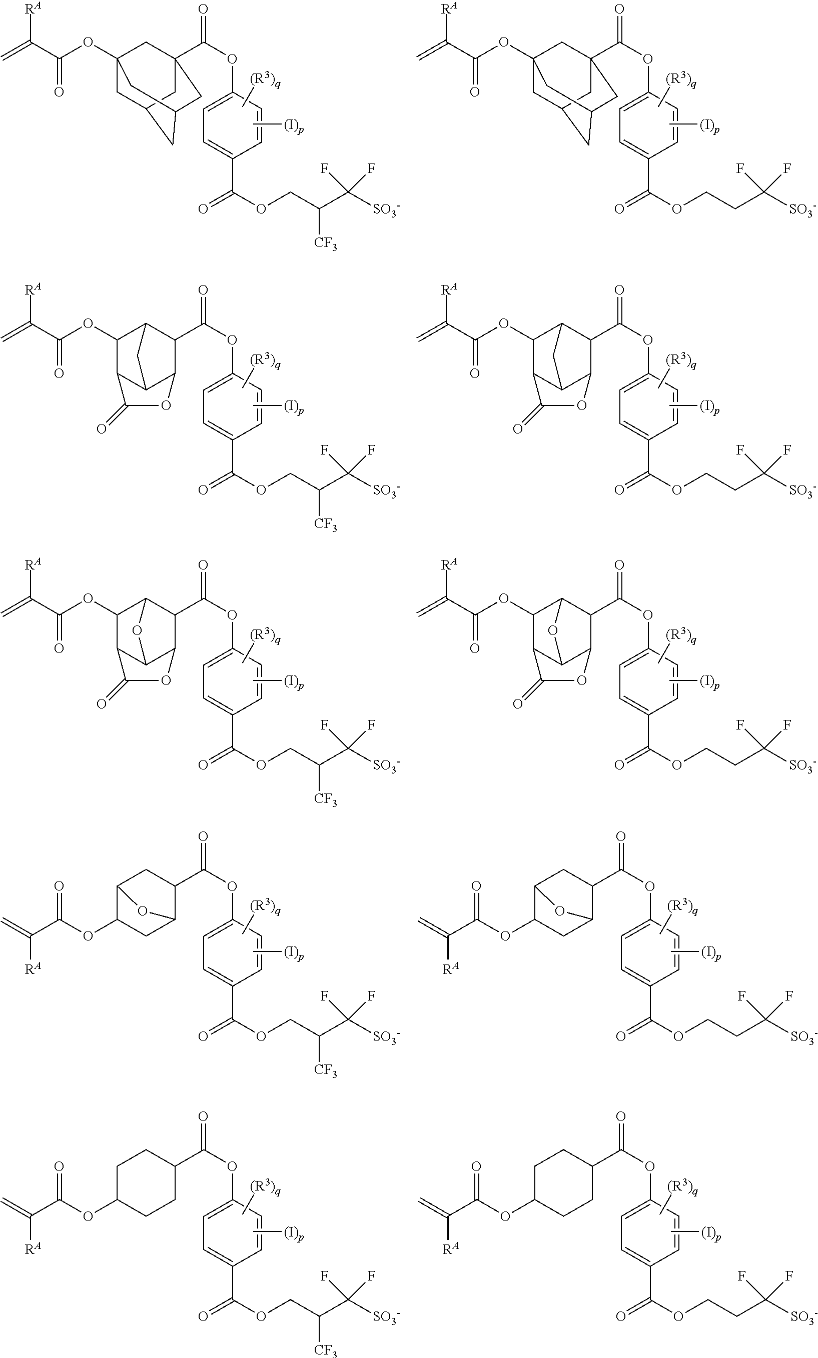

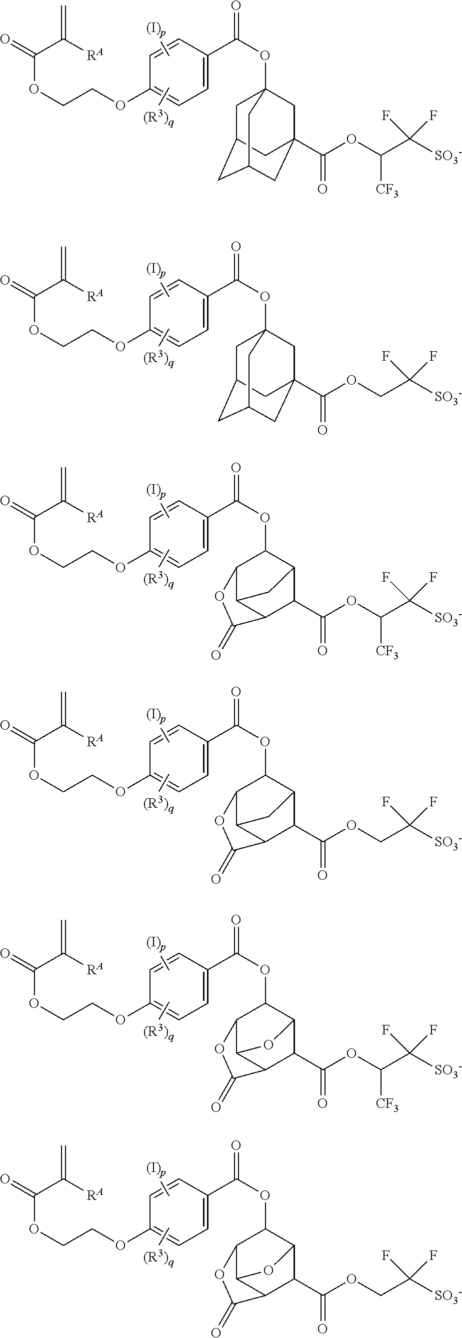

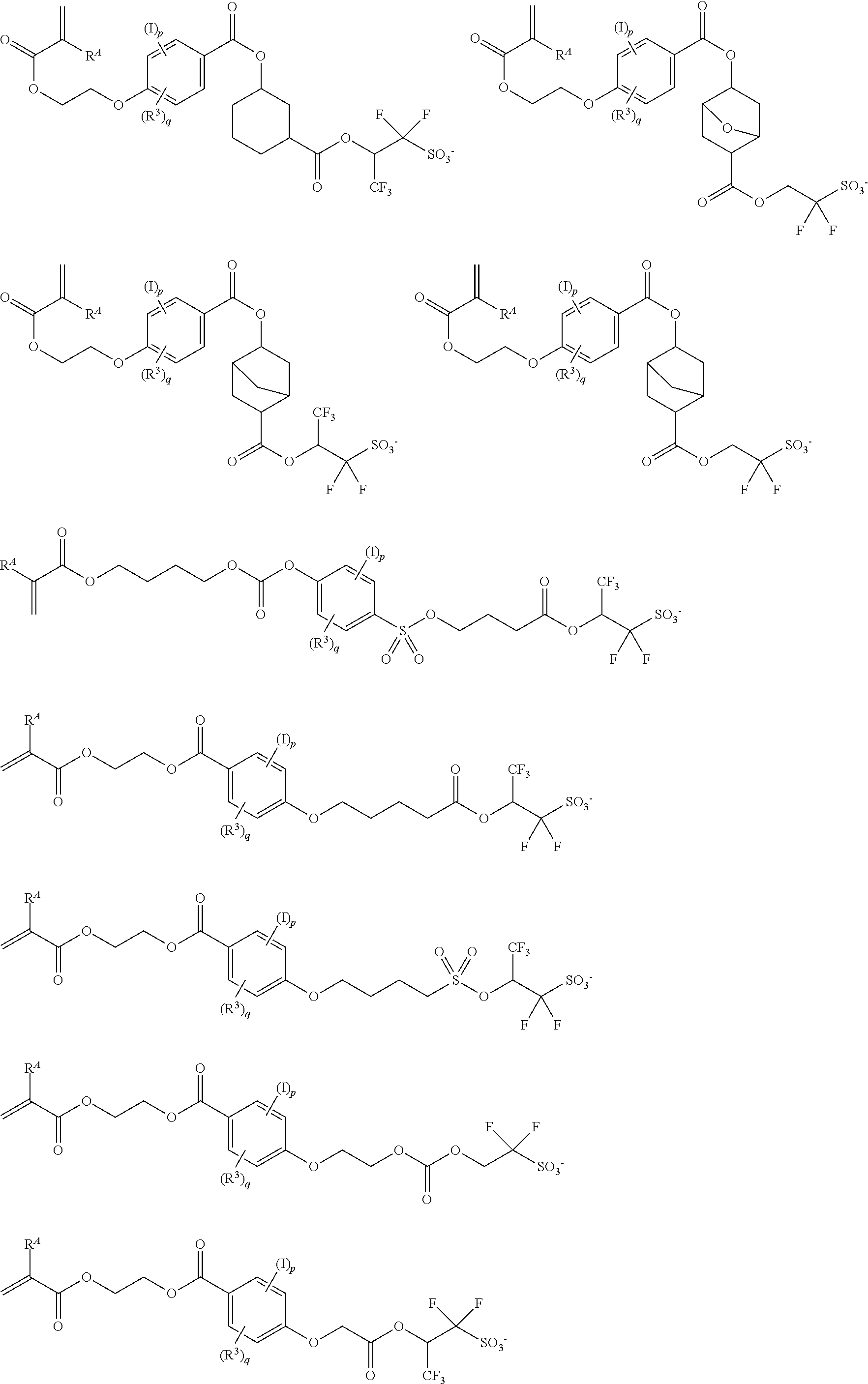

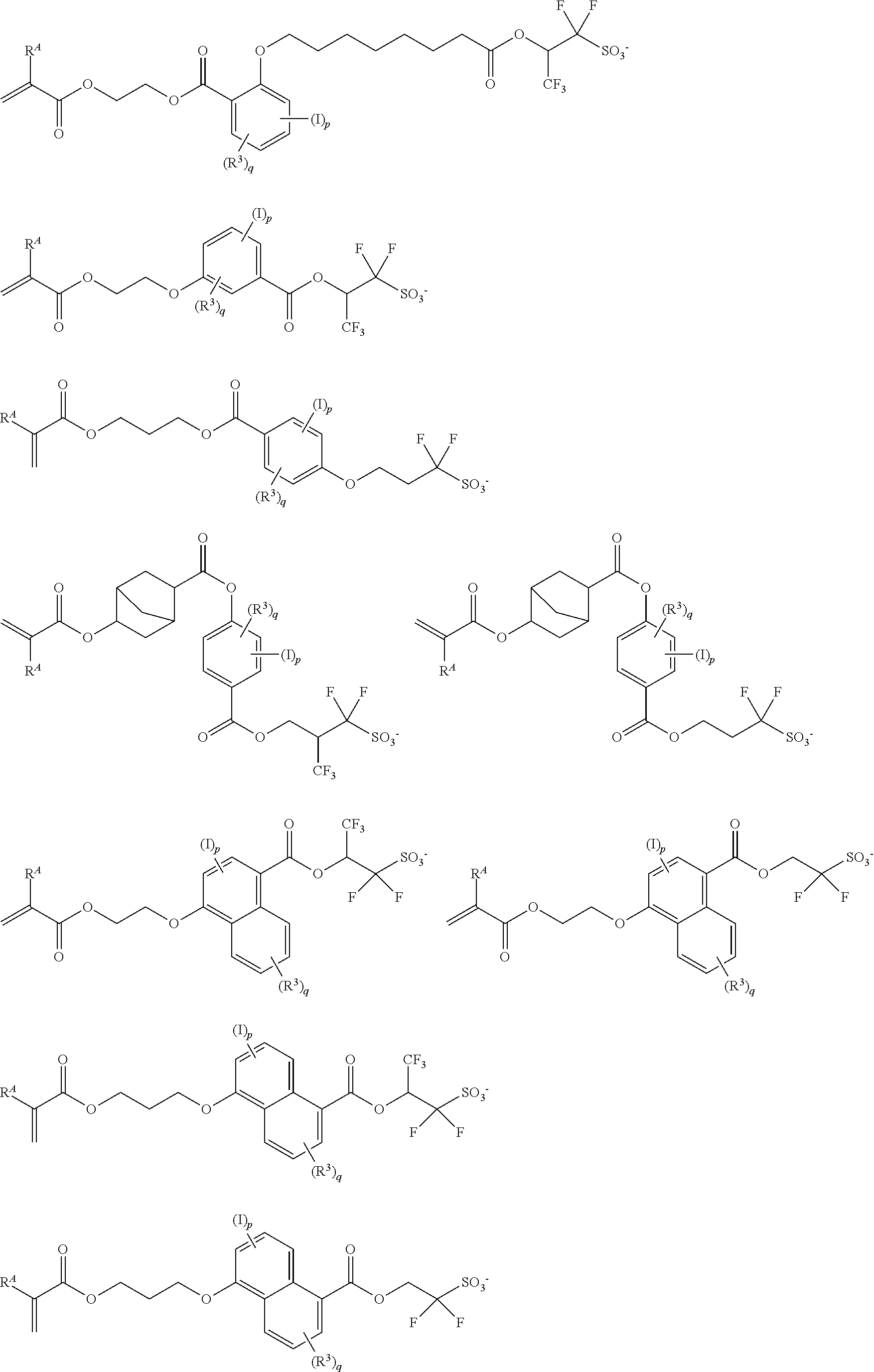

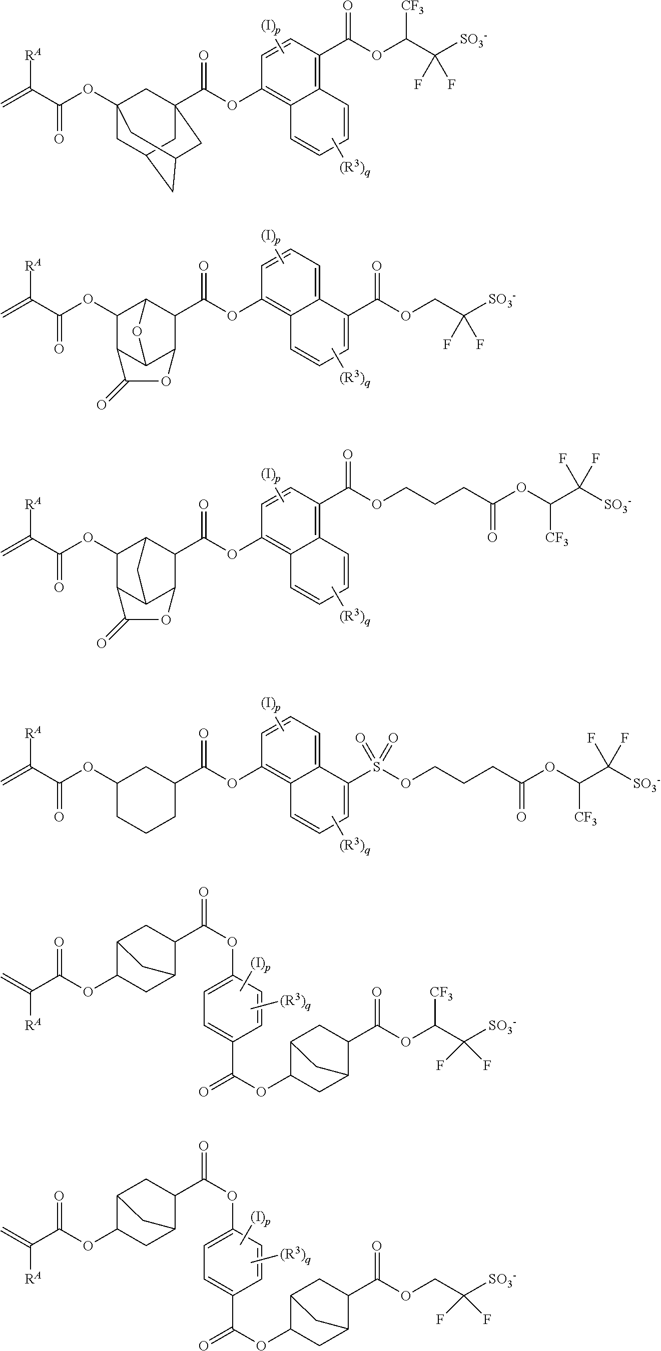

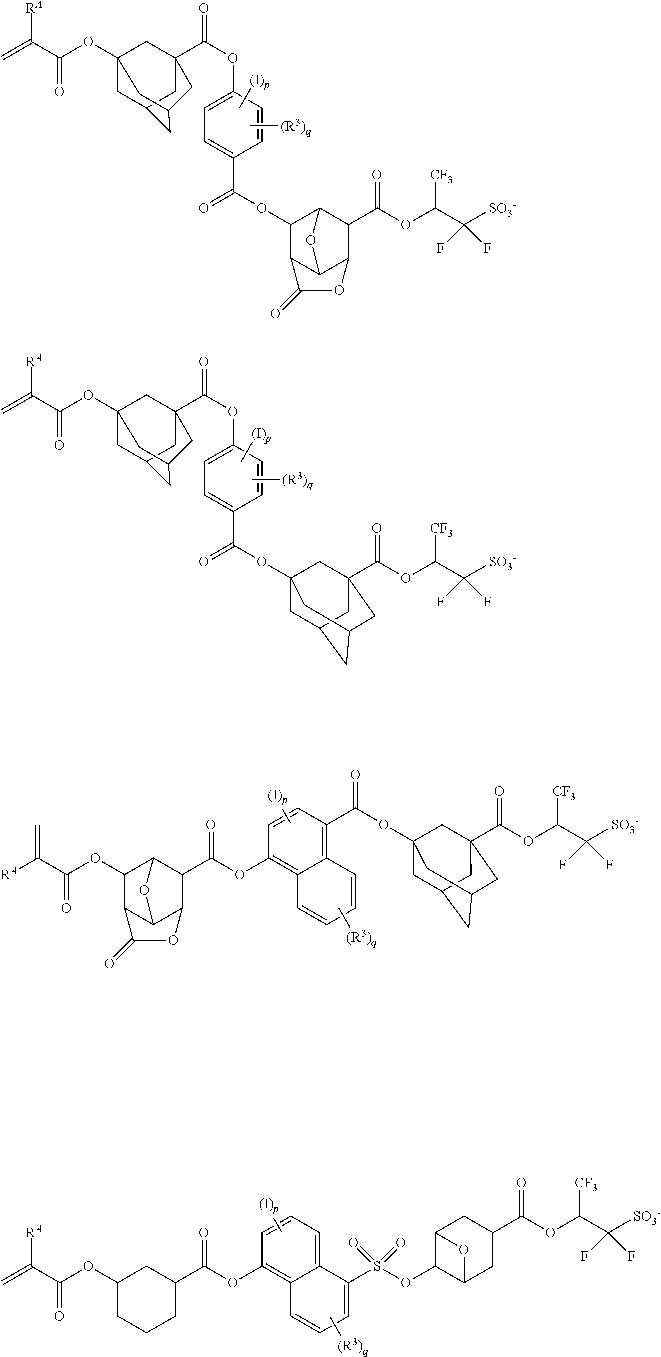

In formula (1). Za⁺ is a sulfonium or iodonium cation.

The sulfonium cation preferably has the formula (Z-1) or (Z-2).

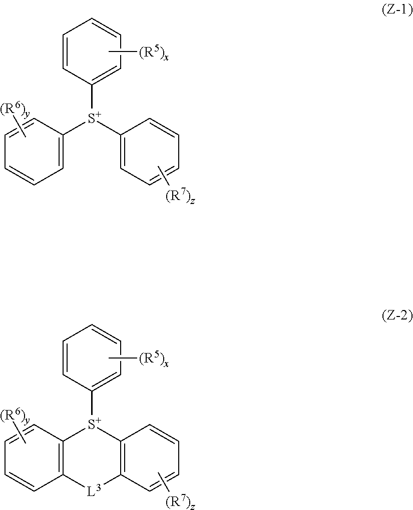

In formulae (Z-1) and (Z-2). R⁵, R⁶, and R⁷ are each independently halogen, hydroxy or a C₁-C₁₅ hydrocarbyl group in which some or all of the hydrogen atoms may be substituted by a moiety containing a heteroatom such as oxygen, sulfur, nitrogen or halogen, and some constituent —CH₂— may be replaced by —O—, —C(═O)—, —S—, —S(═O)—, —S(═O)₂— or —N(R^{N})—. L³ is a single bond, —CH₂—, —O—, —C(═O)—, —S—, —S(═O)—, —S(═O)₂— or —N(R^{N})—. R^{N} is hydrogen or a C₁-C₁₀ hydrocarbyl group in which some or all of the hydrogen atoms may be substituted by a moiety containing a heteroatom such as oxygen, sulfur, nitrogen or halogen, and some constituent —CH₂— may be replaced by —O—, —C(═O)— or —S(═O)₂—.

In formulae (Z-1) and (Z-2), x, y and z are each independently an integer of 0 to 5. When x is 2 or more, a plurality of R⁵ may be identical or different, and two R⁵ may bond together to form a ring with the carbon atoms on the benzene ring to which they are attached. When y is 2 or more, a plurality of R⁶ may be identical or different, and two R⁶ may bond together to form a ring with the carbon atoms on the benzene ring to which they are attached. When z is 2 or more, a plurality of R⁷ may be identical or different, and two R⁷ may bond together to form a ring with the carbon atoms on the benzene ring to which they are attached.

Examples of the sulfonium cation having formula (Z-1) are shown below, but not limited thereto.

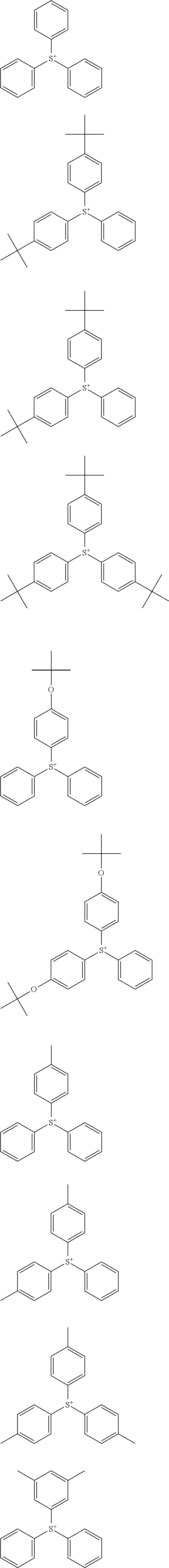

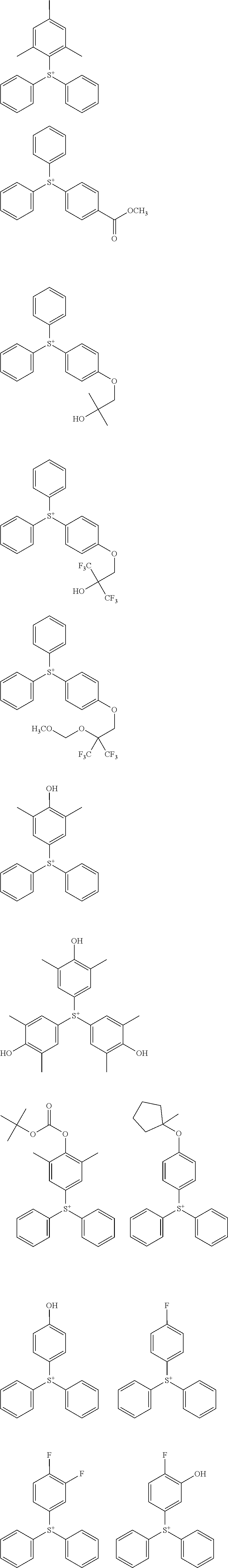

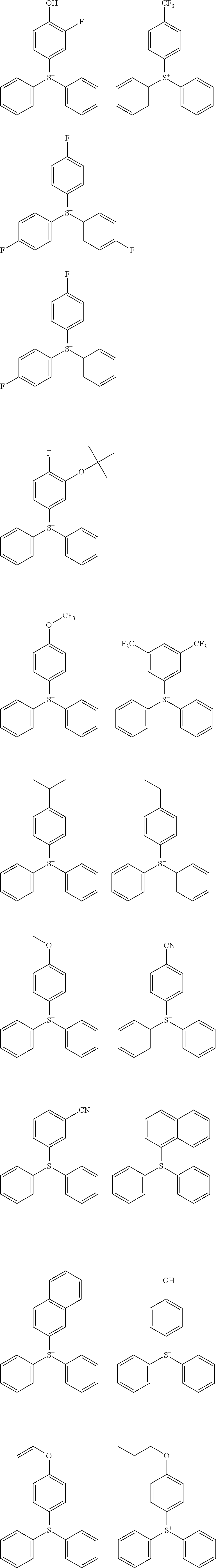

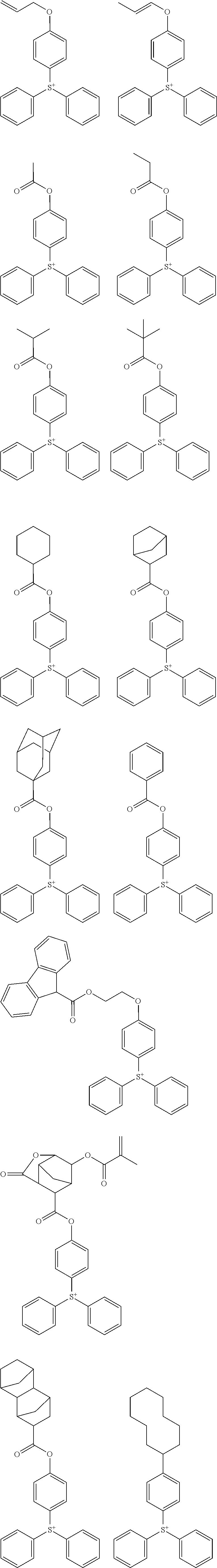

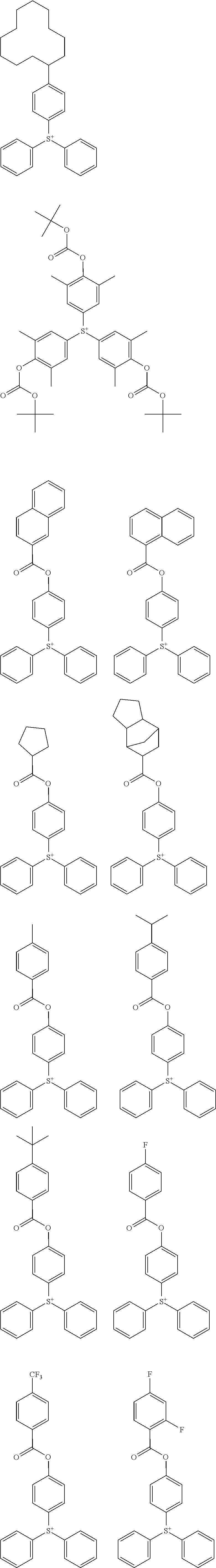

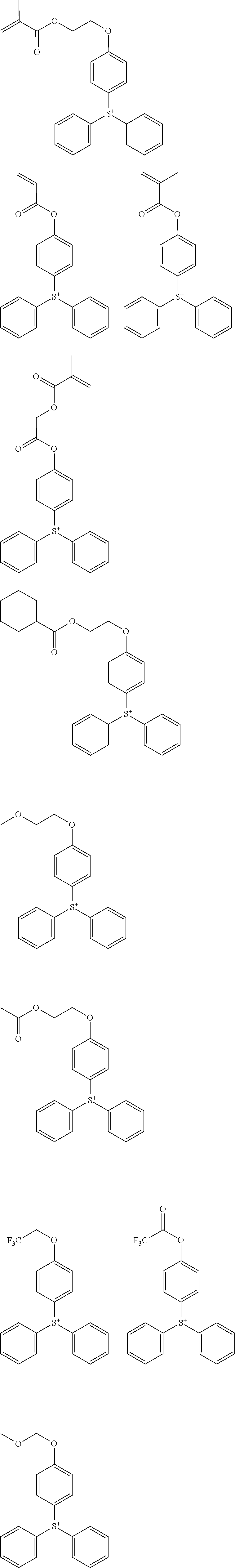

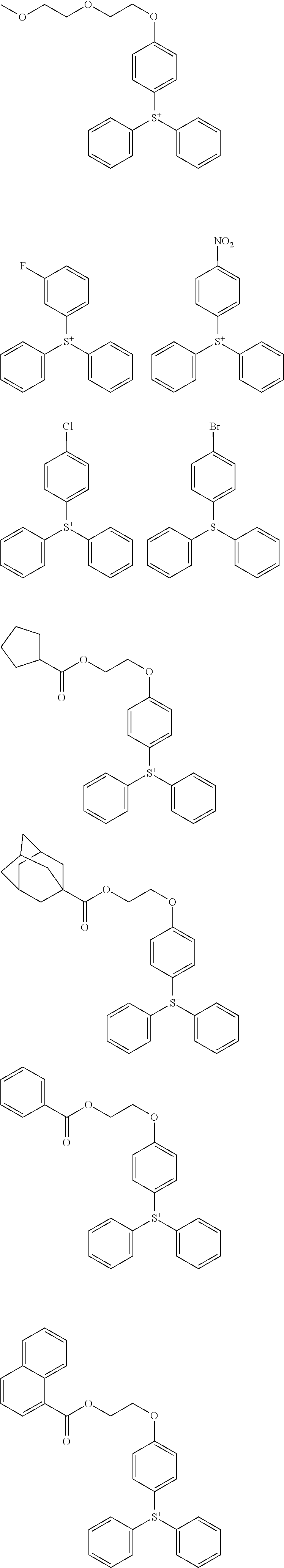

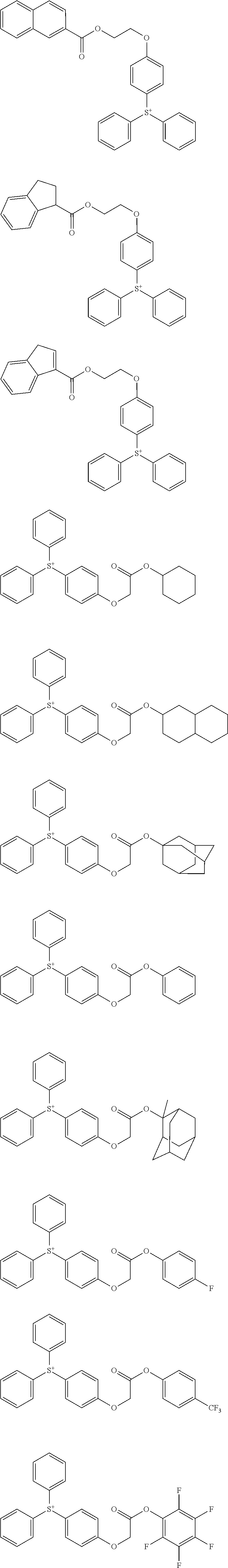

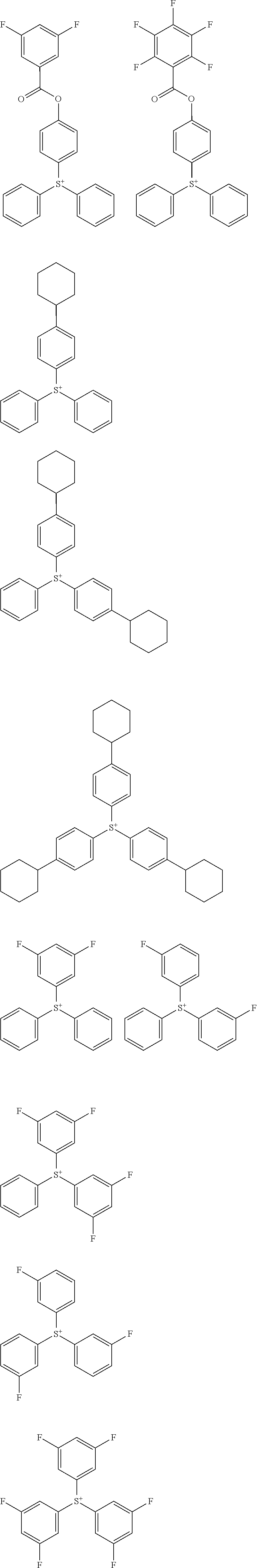

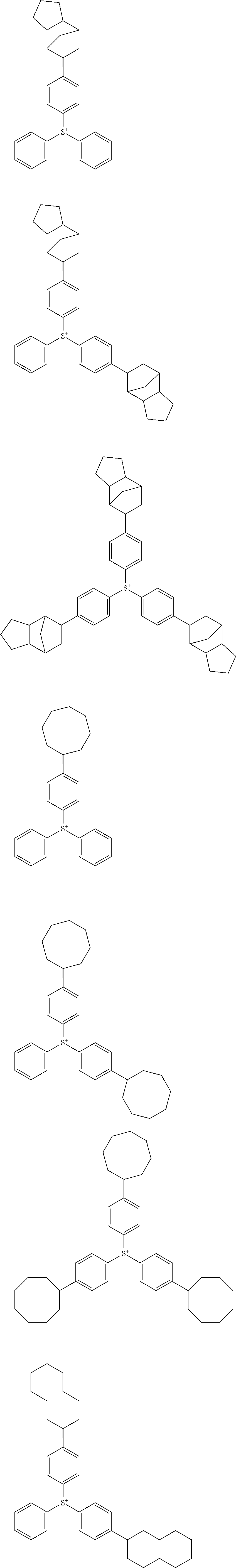

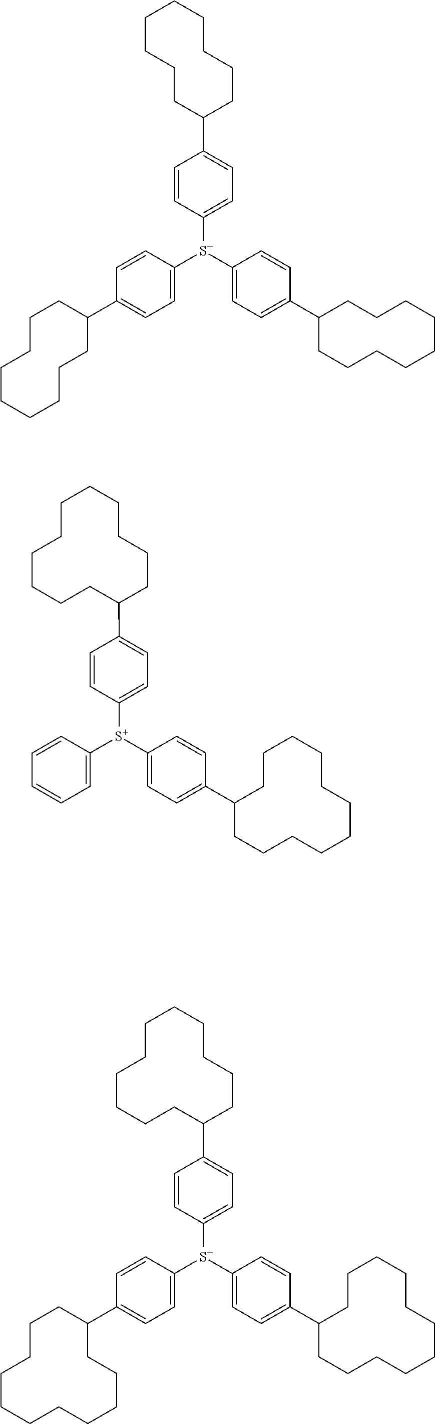 [SMILES: c1cc([S+](c2ccc(C3CCCCCCCCC3)cc2)c2ccc(C3CCCCCCCCC3)cc2)ccc1C1CCCCCCCCC1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

Examples of the sulfonium cation having formula (Z-2) are shown below, but not limited thereto.

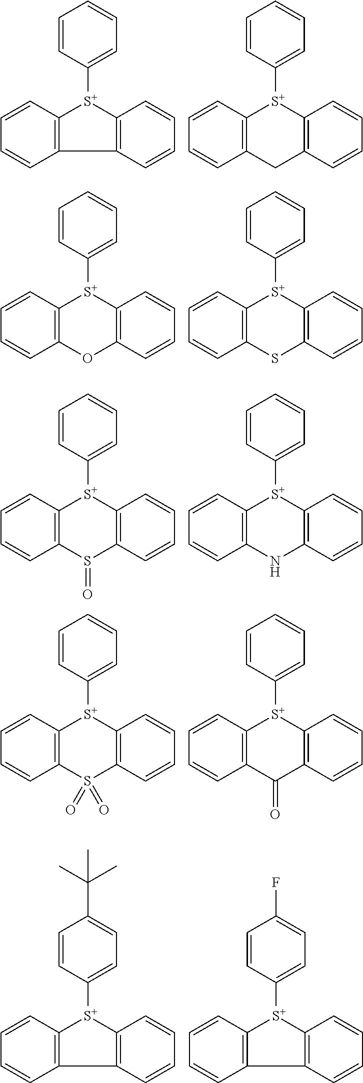

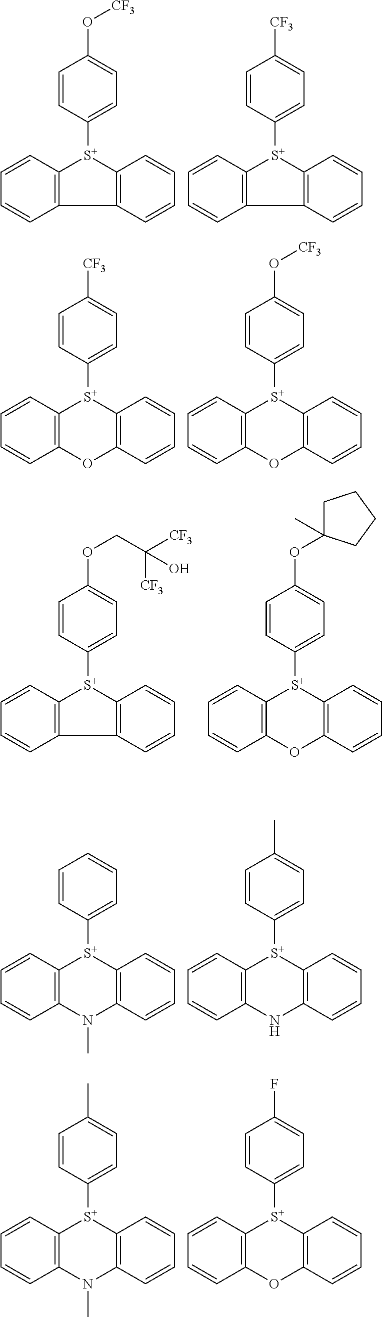

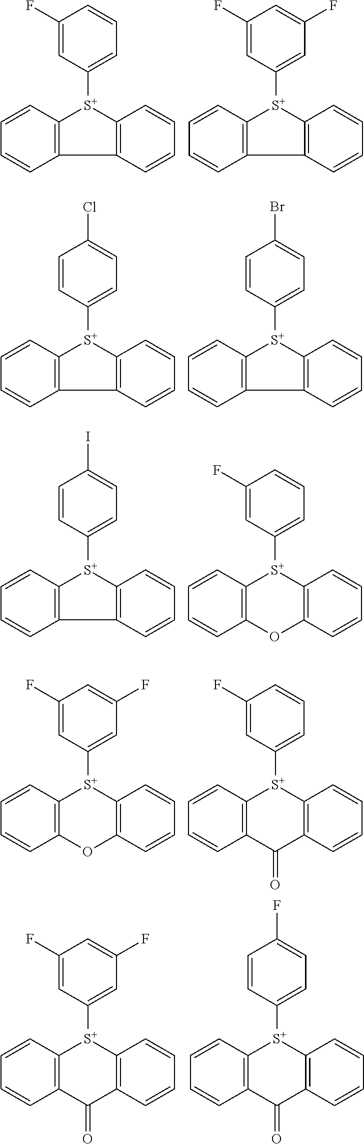

Examples of the iodonium cation include, but are not limited to, diphenyliodonium, bis(4-methylphenyl)iodonium, bis(4-ethylphenyl)iodonium, bis(4-tert-butylphenyl)iodonium, bis(4-(1,1-dimethylpropyl)phenyl)iodonium, 4-methoxyphenylphenyliodoium, 4-tert-butoxyphenylphenyliodonium, 4-acryloyloxyphenylphenyliodonium, 4-methacryloyloxyphenylphenyliodonium, 4-fluorophenylphenyliodonium, and [4-(2-methacryloyloxy-ethoxy)phenyl]phenyliodonium.

Of the onium salt compounds having formula (1), the salts of an anion having formula (1b) with a sulfonium cation having formula (Z-1) or (Z-2) are preferred.

Illustrative structures of the onium salt compound having formula (1) include combinations of anions with cations, both as exemplified above, although the inventive acid generator is not limited thereto.

Among others, combinations of any of the following anions with any of the following cations are most preferred.

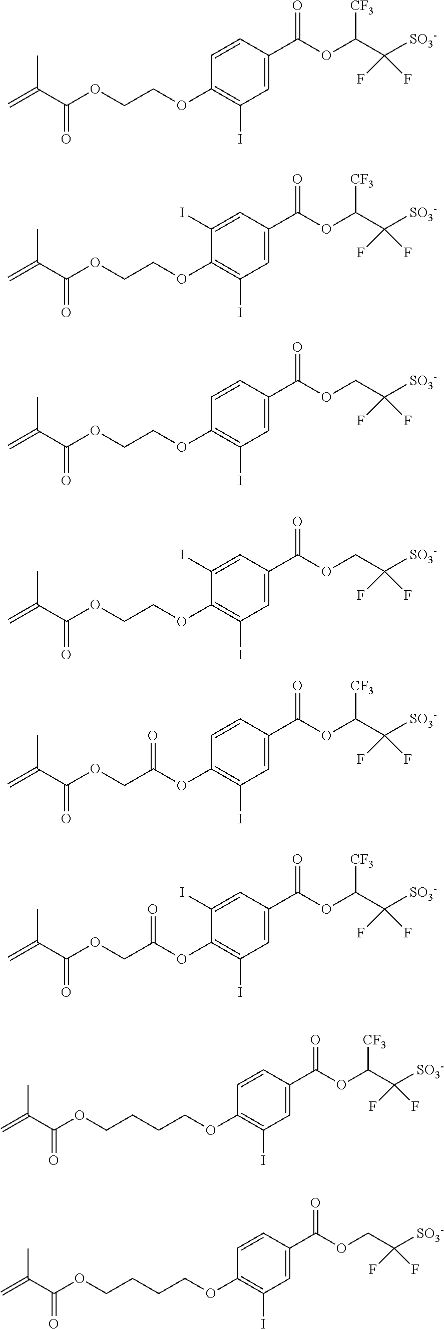

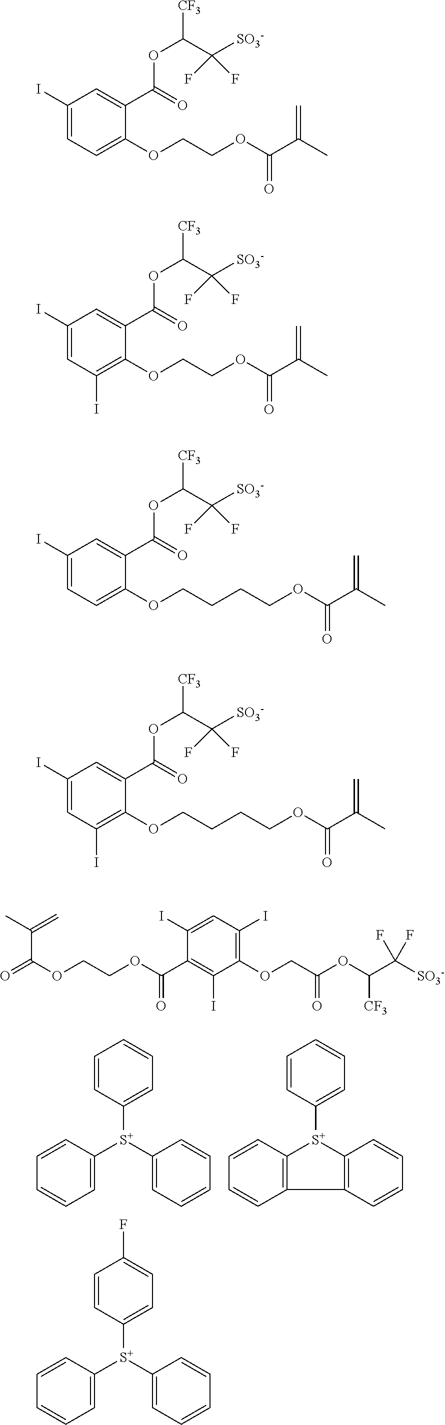

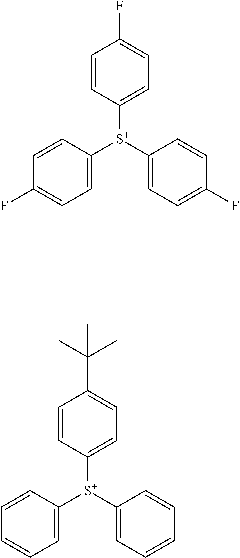 [SMILES: Fc1ccc([S+](c2ccc(F)cc2)c2ccc(F)cc2)cc1 ; CC(C)(C)c1ccc([S+](c2ccccc2)c2ccccc2)cc1]

With respect to the synthesis of the onium salt, for example, the onium salt having formula (1) wherein X¹ and X³ are ester bonds can be synthesized according to the following Scheme 1.

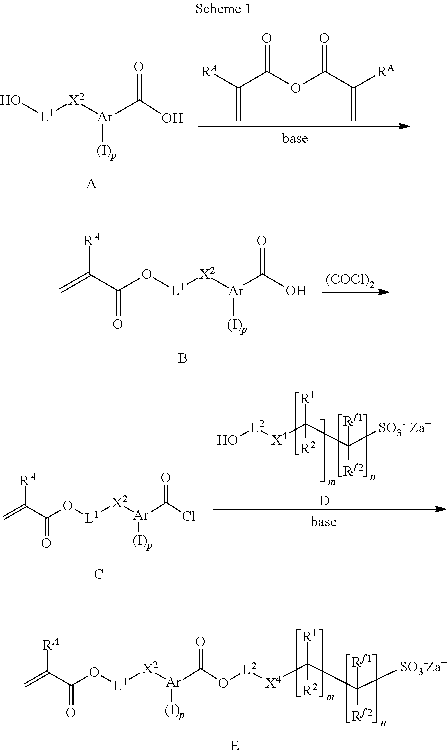

Herein, m, n, p, R¹, R², R^{f1}, R^{f2}, R^{A}, X², X⁴, L¹, L², Ar and Za⁺ are as defined above.

In the first step, hydroxycarboxylic acid A is reacted with methacrylic anhydride or acrylic anhydride and a base to synthesize polymerizable carboxylic acid compound B. In the second step, polymerizable carboxylic acid compound B is reacted with oxalyl chloride to synthesize acid chloride compound C. In the third step, acid chloride compound C is esterified with fluorosulfonium salt D in the presence of a base to synthesize the target compound E. Triethylamine is typical of the base. Alternatively, the target compound can be synthesized by synthesizing fluorosulfonium salt D in the form of an alkali metal salt (whose cation is an alkali metal, e.g., sodium or potassium) or ammonium salt according to Scheme 1, and converting the cation to the desired cation species through ion exchange reaction. It is noted that the ion exchange reaction may be performed by any well-known methods, for example, with reference to JP-A 2007-145797.

Resist Composition

Another embodiment of the invention is a resist composition comprising a base polymer containing a polymer comprising repeat units derived from the onium salt compound and an organic solvent. The polymer is a polymer-bound acid generator, which is effective for substantially suppressing the diffusion of the generated acid. About this concept, several reports are known in the art. For example, Patent Documents 6 and 7 disclose resist compositions comprising a polymer comprising repeat units having a photoacid generator of specific anion structure incorporated therein. These resist compositions, however, are poor in lithography properties such as sensitivity, MEF, LWR, and CDU as compared with the resist composition comprising the inventive polymer as the base polymer.

The resist composition adapted for the EUV lithography must achieve a high sensitivity, high resolution and low LWR at the same time. It is important to overcome the tradeoff relationship that as the distance of acid diffusion is shortened, LWR is reduced, but the sensitivity becomes lower. The polymer described in Patent Document 7 and the polymer comprising repeat units derived from the inventive onium salt compound contain iodine. Since the iodine atom is highly absorptive to EUV of wavelength 13.5 nm, it generates secondary electrons upon exposure. The energy of secondary electrons is transferred to the acid generator to promote its decomposition, contributing to a higher sensitivity despite low acid diffusion.

However, Patent Document 7 refers nowhere to a carbon chain between a polymerizable group and an iodized group. Due to the robust structure that these groups are directly bonded, the freedom of sulfonic acid in the polymer is restrained. Since the sulfonic acid is localized within the polymer, despite low acid diffusion, satisfactory LWR is not obtained in the formation of small size patterns. It is also a problem that the robust structure leads to high crystallinity and low solvent solubility.

The onium salt compound of the invention is characterized by the structure that a polymerizable group is linked to an iodized aromatic group via a carbon chain having at least two carbon atoms. Since the inventive polymer has the anion attached to the main chain and contains iodine with a high atomic weight, the acid diffusion in the resist composition after exposure is suppressed. Another more important characteristic is that since a carbon chain intervenes in the structure, the degree of freedom of sulfonic acid in the polymer is high. Since the acid generator is uniformly dispersed in the polymer and bound to the polymer main chain by mixing the acid generator prior to polymerization, the sulfonic acid site of the acid generator moves while being bound to the polymer main chain, by virtue of the high degree of freedom due to the carbon chain. Then, appropriate acid diffusion takes place in the exposed range. Due to appropriate control of acid diffusion, LWR and CDU are significantly improved. Further, this distribution state of sulfonic acid promotes acid elimination reaction in the polymer, leading to an improvement in sensitivity. The structure having a highly lipophilic carbon chain also contributes to an improvement in solvent solubility. For these reasons, the onium salt compound of the invention is best suited for forming small size patterns.

The inventive polymer exerts the effects of improving sensitivity, LWR and CDU, which stands good either in positive and negative tone pattern formation by aqueous alkaline development or in negative tone pattern formation by organic solvent development.

When the inventive polymer further comprises repeat units having a phenolic hydroxy group and repeat units having an acid labile group containing a fluorinated aromatic ring and capable of generating a tertiary benzyl cation, lithography properties are further improved. Upon light exposure, the repeat unit having a phenolic hydroxy group generates secondary electrons, which are effectively conducted to the cation of the inventive photoacid generator to promote decomposition of the salt to generate the corresponding acid in an efficient manner. As described above, no excessive acid diffusion take place at this point of time. On the other hand, the repeat unit having an acid labile group containing a fluorinated aromatic ring and capable of generating a tertiary benzyl cation exhibits a higher reactivity with acid because the tertiary benzyl cation created after elimination reaction is more stable than the carbo cation eliminated from the conventional acid labile group of tertiary ester form. This leads to improvements in sensitivity and dissolution contrast in developer. Presumably, it becomes possible to improve solvent solubility and to suppress agglomeration of polymer chains by increasing the concentration of fluorine in the polymer. The combination of these repeat units ensures to form patterns with a high sensitivity, high contrast, reduced LWR, and improved CDU.

Base Polymer

The invention also provides a polymer comprising repeat units derived from the inventive onium salt compound, referred to as repeat units (a), hereinafter, preferably repeat units derived from the onium salt compound having formula (1b).

The polymer may also function as a base polymer. In the case of a chemically amplified positive resist composition, the polymer further comprises repeat units having an acid labile group, preferably repeat units having the formula (b1) or repeat units having the formula (b2). These units are also referred to as repeat units (b1) and (b2), respectively.

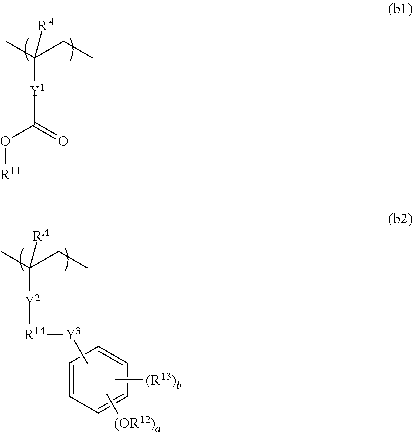

In formulae (b1) and (b2), R^{A} is each independently hydrogen or methyl. Y¹ is a single bond, phenylene, naphthylene, or a C₁-C₁₂ linking group containing at least one moiety selected from ester bond and lactone ring. Y² is a single bond or ester bond. Y³ is a single bond, ether bond or ester bond. R¹¹ and R¹² are each independently an acid labile group. When the polymer contains both repeat units (b1) and (b2), R¹¹ and R¹² may be the same or different. R¹³ is fluorine, trifluoromethyl, cyano or a C₁-C₆ saturated hydrocarbyl group. R¹⁴ is a single bond or a C₁-C₆ alkanediyl group in which some —CH₂— may be replaced by an ether bond or ester bond. The subscript “a” is 1 or 2, “b” is an integer of 0 to 4, and a+b is from 1 to 5.

Examples of the monomer from which repeat units (b1) are derived are shown below, but not limited thereto. R^{A} and R¹¹ are as defined above.

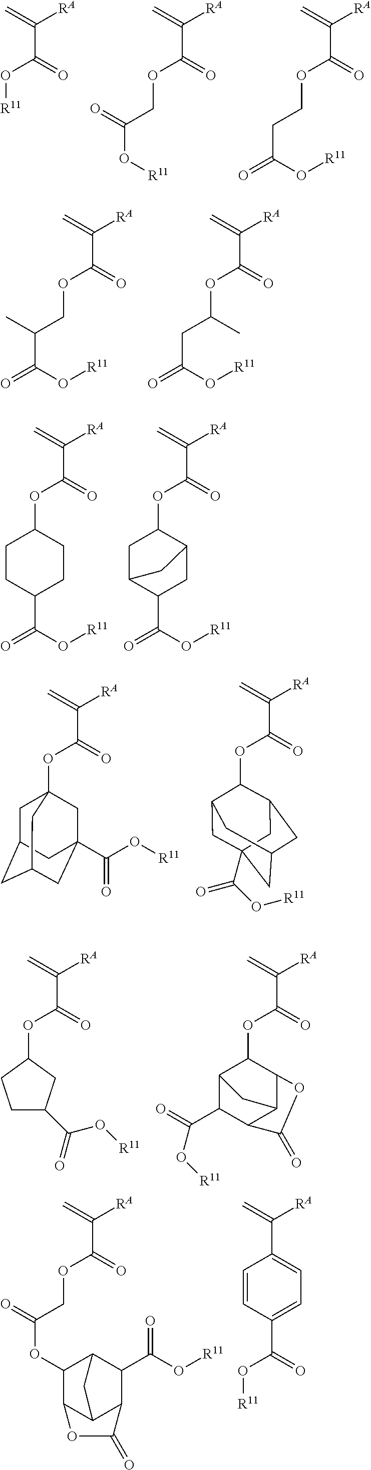

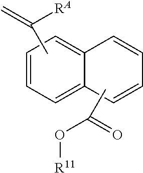

Examples of the monomer from which repeat units (b2) are derived are shown below, but not limited thereto. R^{A} and R¹² are as defined above.

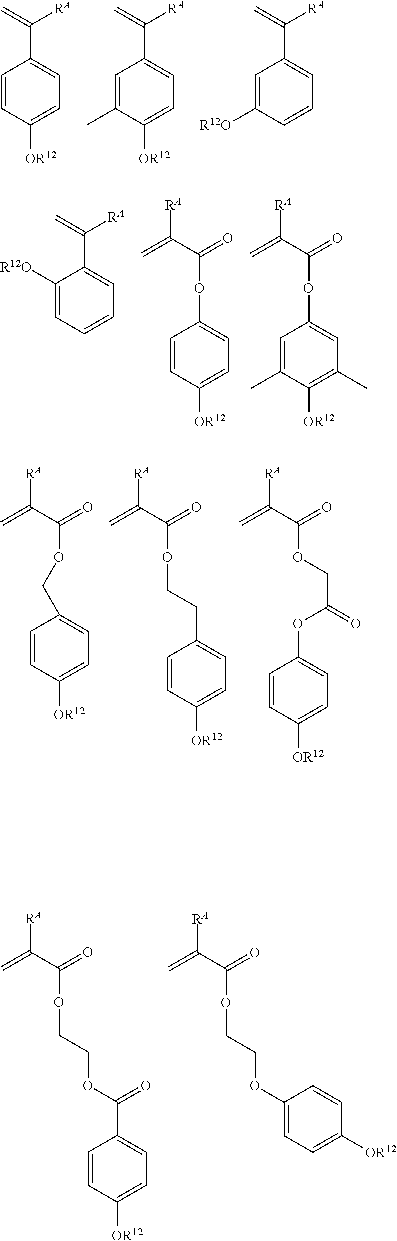

The acid labile groups represented by R¹¹ and R¹² in formulae (b1) and (b2) may be selected from a variety of such groups, for example, those groups described in JP-A 2013-080033 (U.S. Pat. No. 8,574,817) and JP-A 2013-083821 (U.S. Pat. No. 8,846,303).

Typical of the acid labile group are groups of the following formulae (AL-1) to (AL-3).

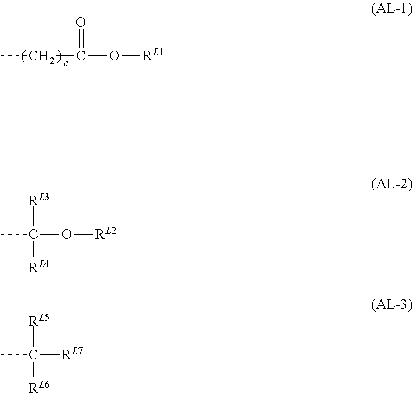

In formulae (AL-1) and (AL-2), R^{L1} and R^{L2} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. Preferred are C₁-C₄₀ saturated hydrocarbyl groups, with C₁-C₂₀ saturated hydrocarbyl groups being more preferred.

In formula (AL-1), c is an integer of 0 to 10, preferably 1 to 5.

In formula (AL-2), R^{L3} and R^{L4} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ saturated hydrocarbyl groups are preferred. Any two of R^{L2}, R^{L3} and R^{L4} may bond together to form a C₃-C₂₀ ring with the carbon atom or carbon and oxygen atoms to which they are attached, the ring being preferably of 4 to 16 carbon atoms and more preferably alicyclic.

In formula (AL-3), R^{L5}, R^{L6} and R^{L7} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ saturated hydrocarbyl groups are preferred. Any two of R^{L5}, R^{L6} and R^{L7} may bond together to form a C₃-C₂₀ ring with the carbon atom to which they are attached, the ring being preferably of 4 to 16 carbon atoms and more preferably alicyclic.

Of the acid labile groups having formula (AL-3), those having the formula (AL-4) are also preferred.

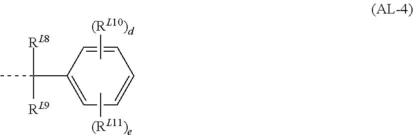

In formula (AL-4), R^{L8} and R^{L9} are each independently a C₁-C₁₀ hydrocarbyl group which may contain a heteroatom. R^{L8} and R^{L9} may bond together to form a ring with the carbon atom to which they are attached. R^{L10} is fluorine, a C₁-C₅ fluorinated alkyl group, or a C₁-C₅ fluorinated alkoxy group. R^{L11} is a C₁-C₁₀ hydrocarbyl group which may contain a heteroatom. The subscript d is 1 or 2, e is an integer of 0 to 5, and d+e is from 1 to 5.

Examples of the acid labile group having formula (AL-4) are shown below, but not limited thereto.

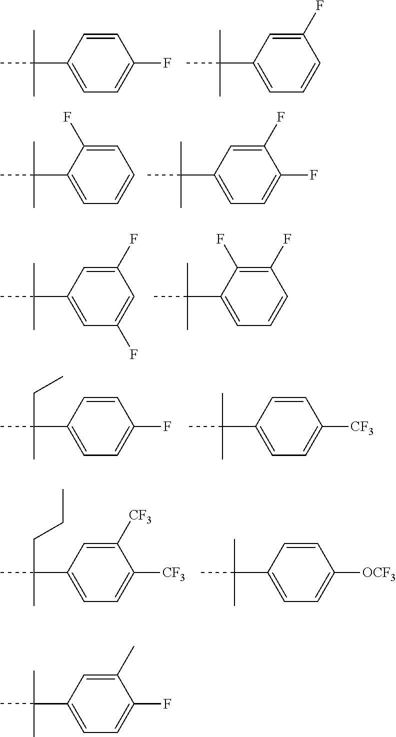

Where the polymer also functions as a base polymer, it may further comprise repeat units (c) having a phenolic hydroxy group as an adhesive group. Examples of the monomer from which repeat units (c) are derived are shown below, but not limited thereto. Herein, R^{A} is as defined above.

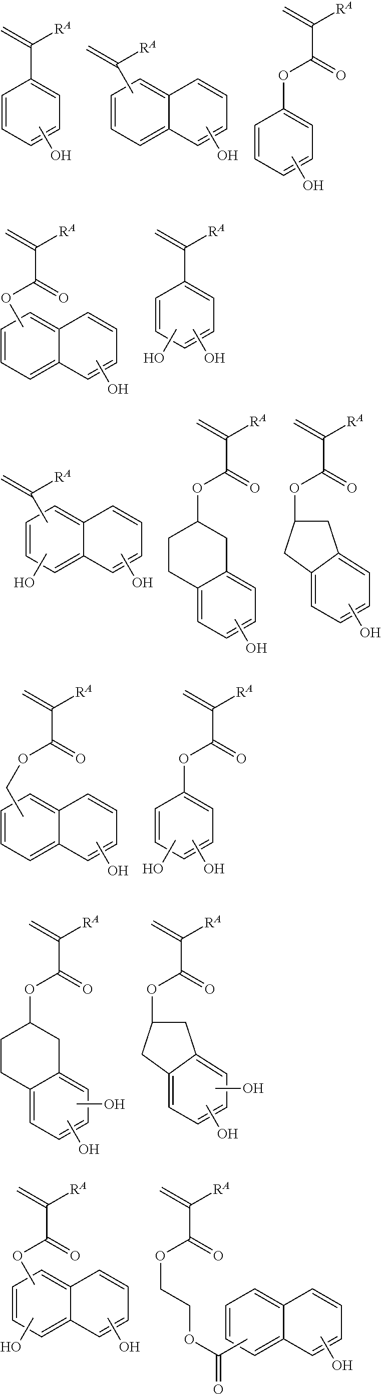

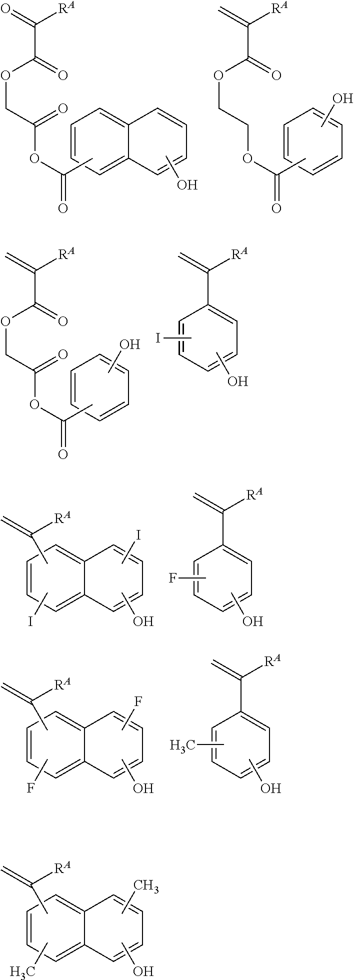

Where the polymer also functions as a base polymer, it may further comprise repeat units (d) having another adhesive group selected from hydroxy group (other than the foregoing phenolic hydroxy), carboxy group, lactone ring, sultone ring, ether bond, ester bond, carbonyl group, sulfonyl group, and cyano group. Examples of the monomer from which repeat units (d) are derived are given below, but not limited thereto. Herein R^{A} is as defined above.

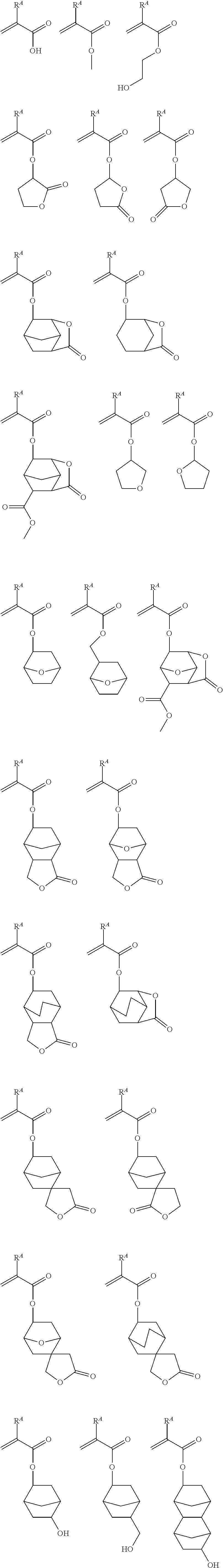

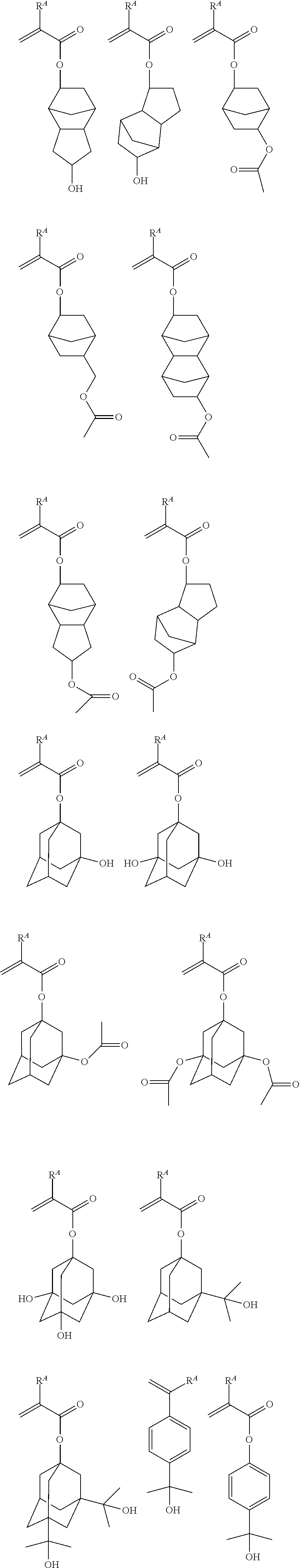

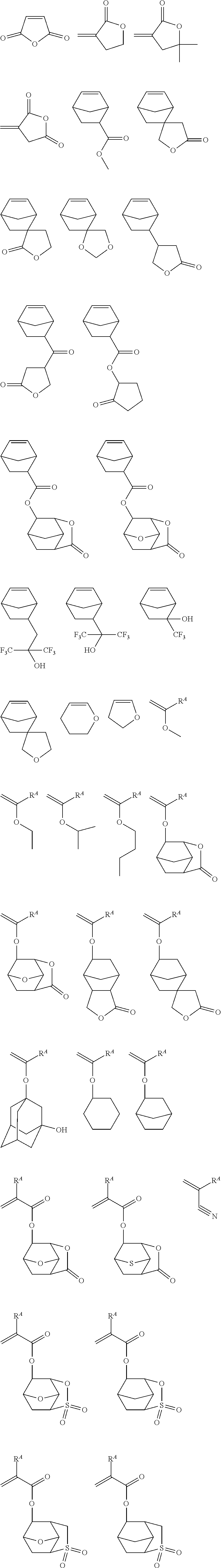

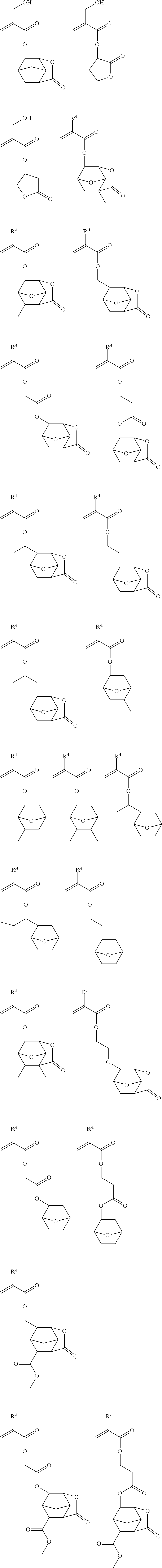

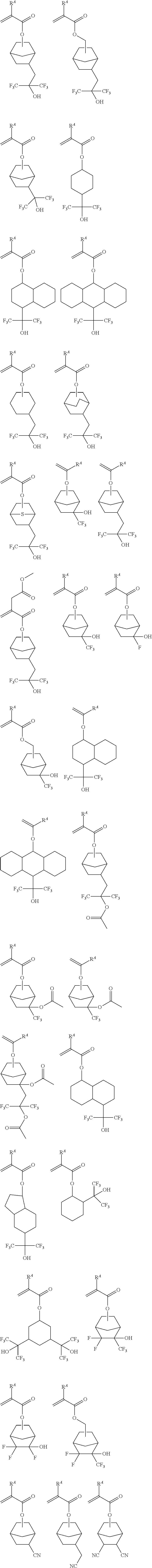

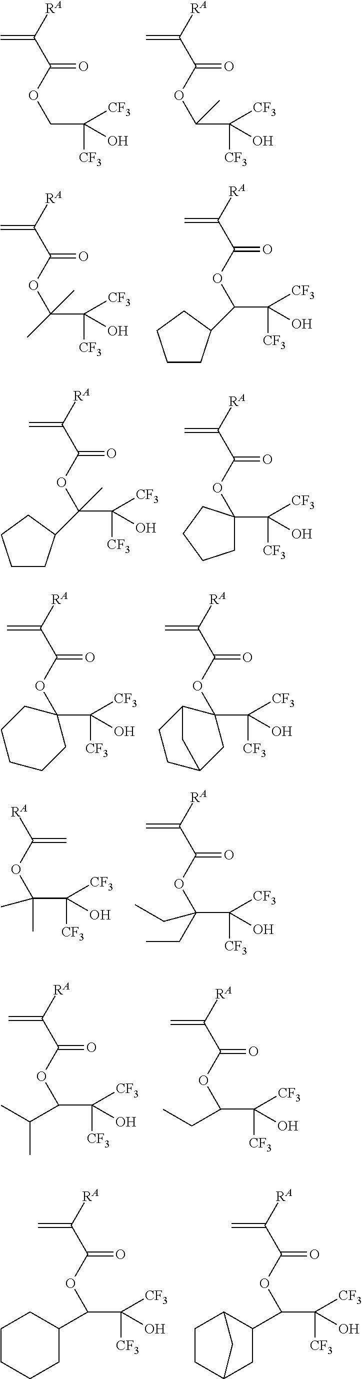

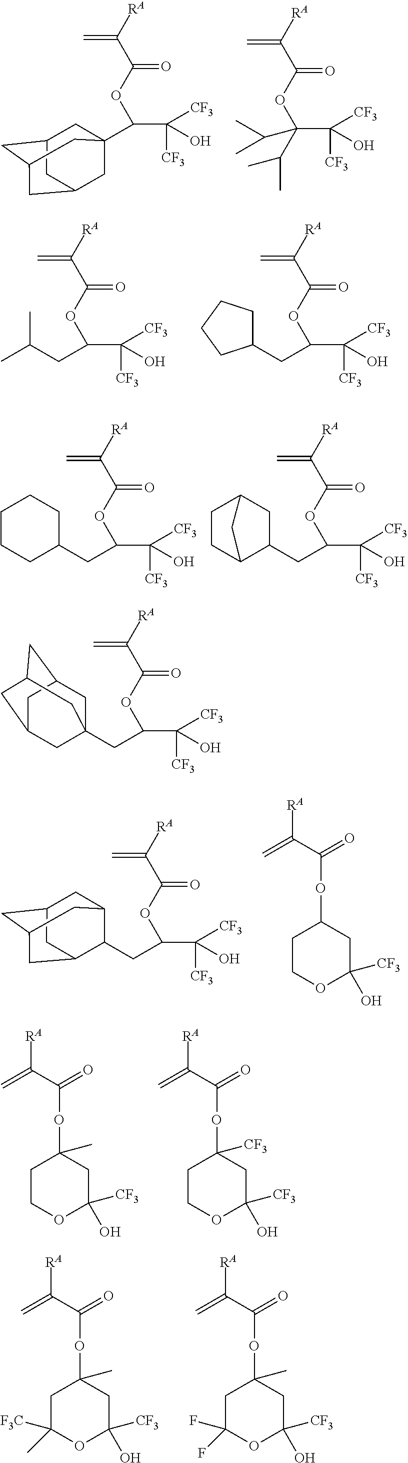

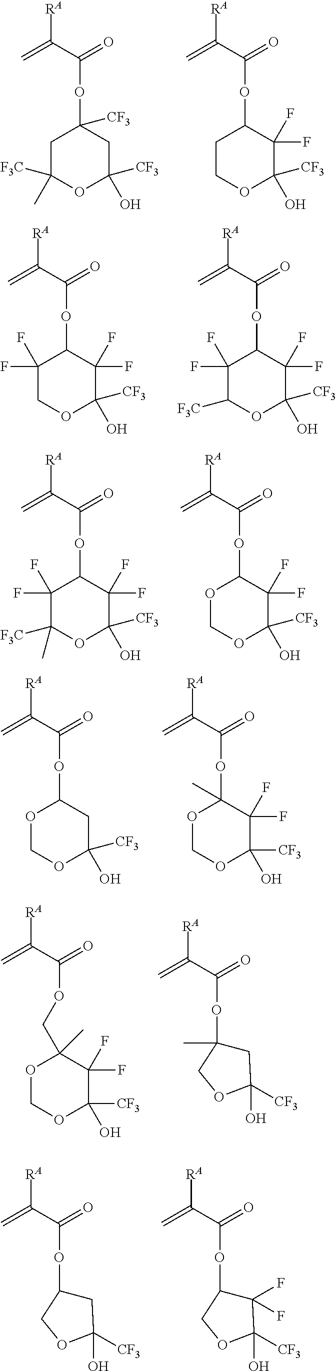

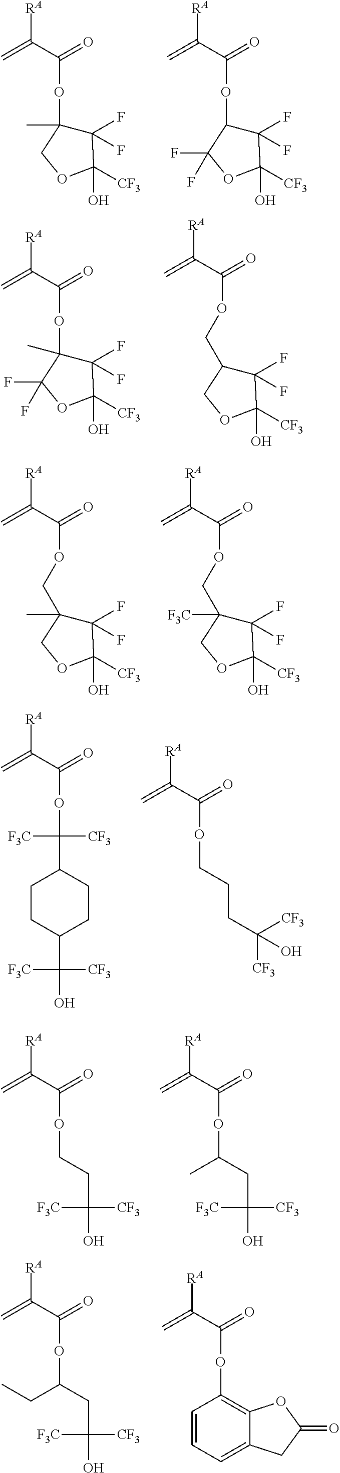

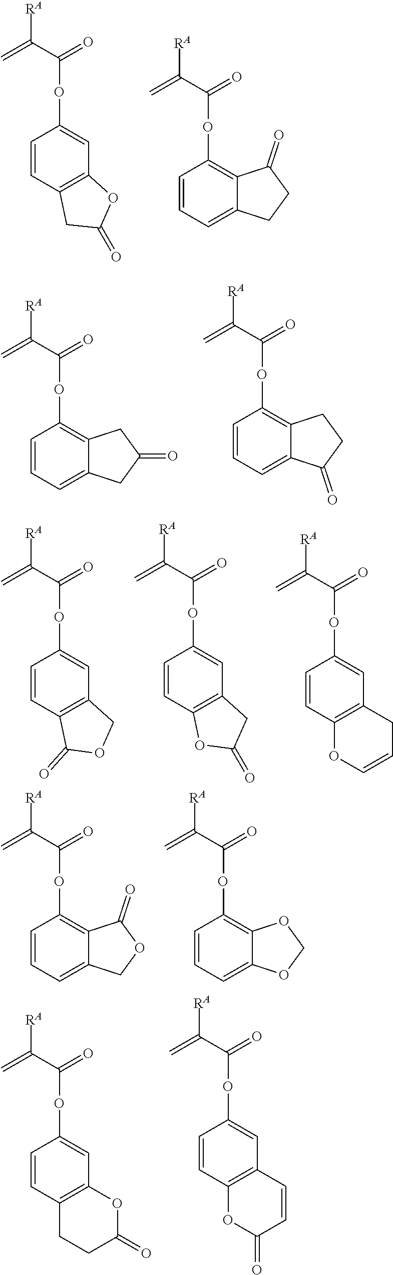

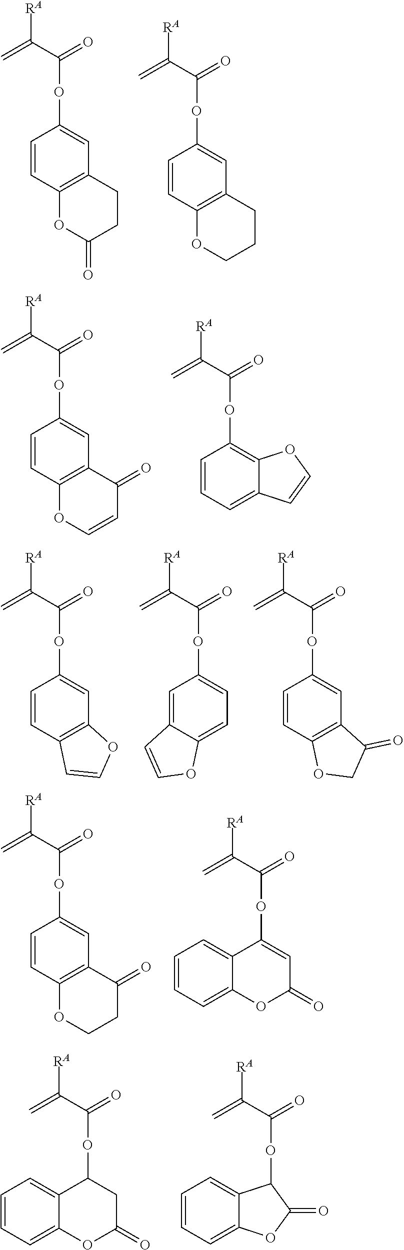

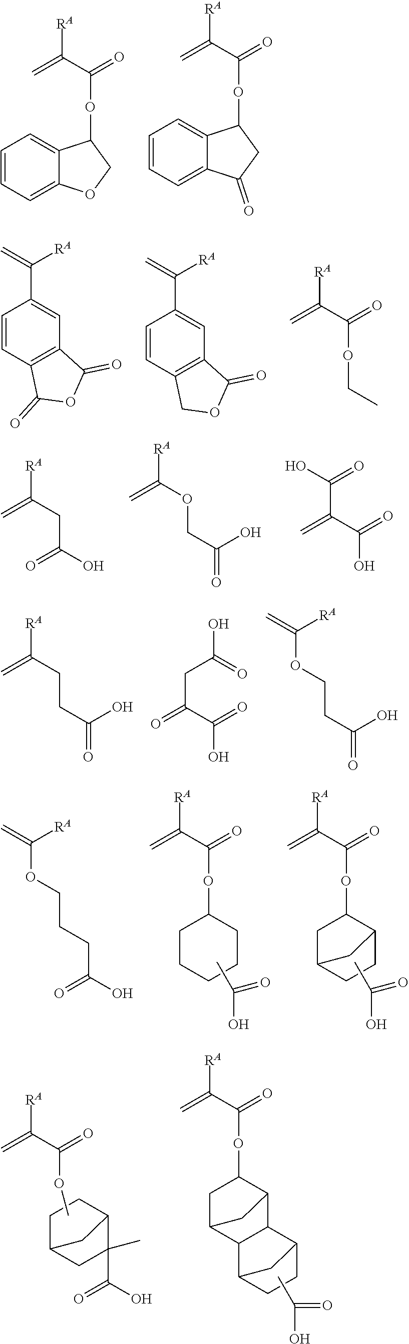

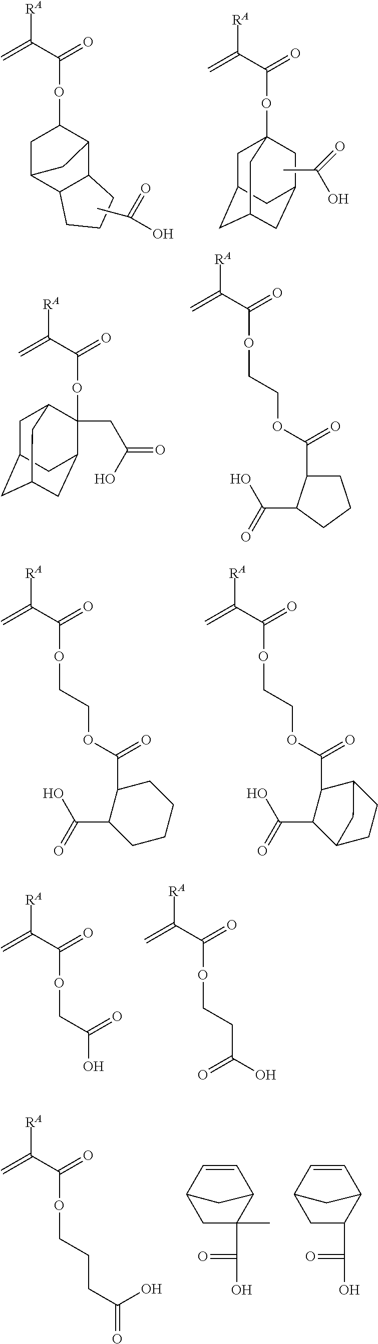

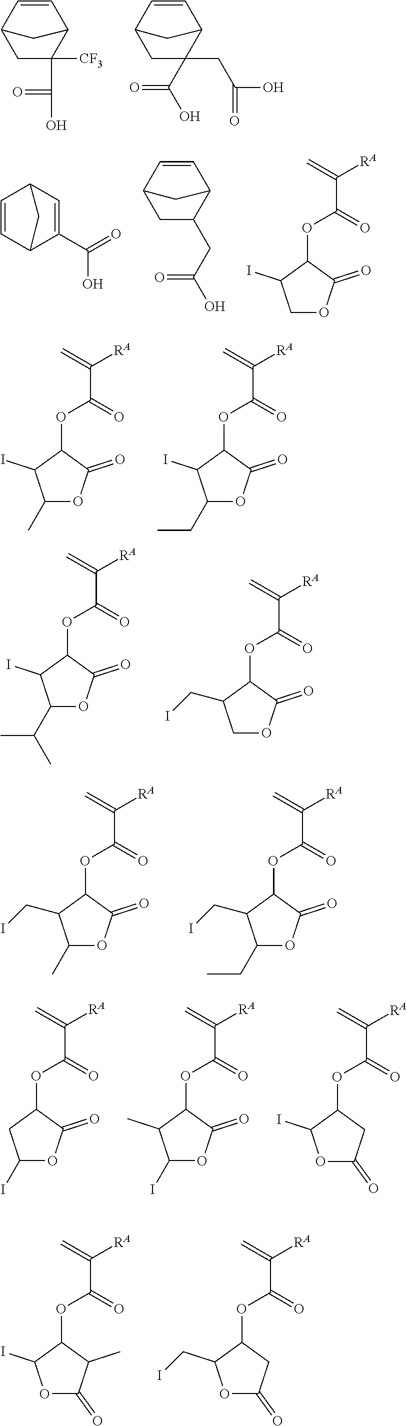

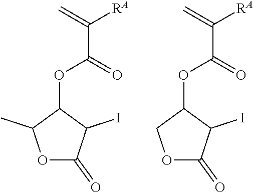

Where the polymer also functions as a base polymer, it may further comprise repeat units (e) derived from indene, benzofuran, benzothiophene, acenaphthylene, chromone, coumarin, norbornadiene, or derivatives thereof. Examples of the monomer from which repeat units e are derived are given below, but not limited thereto.

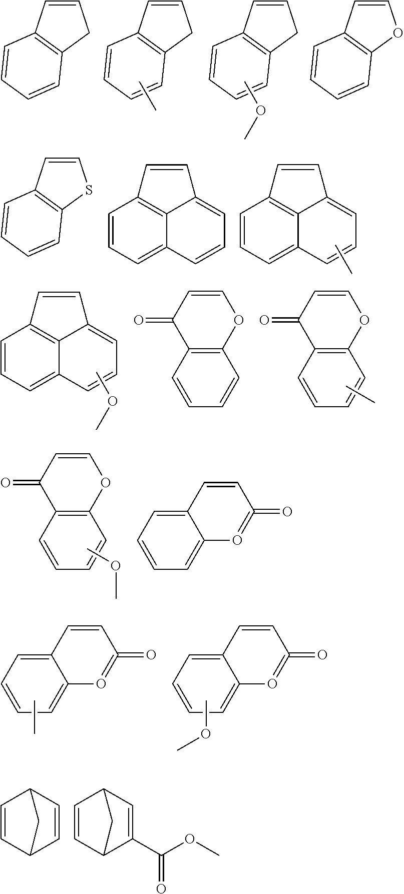

Where the polymer also functions as a base polymer, it may further comprise repeat units (f) derived from indane, vinylpyridine, vinylcarbazole, or derivatives thereof.

The polymer may further comprise repeat units (g) derived from an onium salt containing a polymerizable unsaturated bond, other than repeat units (a). Examples of repeat units (g) are described in JP-A 2017-008181, paragraph [0060].

The base polymer for formulating the positive resist composition comprises repeat units (a) and repeat units (b1) and/or (b2) having an acid labile group as essential components and additional repeat units (c), (d), (e), (f), and (g) as optional components. A fraction of units (a), (b1), (b2), (c), (d), (e), (f), and (g) is:
preferably 0<a<1.0, 0≤b1<1.0, 0≤b2<1.0, 0<b1+b2<1.0, 0≤c≤0.9, 0≤d≤0.9, 0≤e≤0.8, 0≤f≤0.8, and 0≤g≤0.4;
more preferably 0.02≤a≤0.7, 0≤b1≤0.9, 0≤b2≤0.9, 0.1≤b1+b2≤0.9, 0≤c≤0.8, 0≤d≤0.8, 0≤e≤0.7, 0≤f≤0.7, and 0≤g≤0.3; and
even more preferably 0.03≤a≤0.5, 0≤b1≤0.8, 0≤b2≤0.8, 0.1≤b1+b2≤0.8, 0≤c≤0.7, 0≤d≤0.7, 0≤e≤0.6, 0≤f≤0.6, and 0≤g≤0.2. Notably, a+b1+b2+c+d+e+f+g=1.0.

For the base polymer for formulating the negative resist composition, an acid labile group is not necessarily essential. The base polymer comprises essentially repeat units (a) and optionally repeat units (c), (d), (e), (f) and/or (g). A fraction of these units is:
preferably 0<a<1.0, 0≤c≤1.0, 0≤d≤0.9, 0≤e≤0.8, 0≤f≤0.8, and 0≤g≤0.4;
more preferably 0.02≤a≤0.7, 0.2≤c≤1.0, 0≤d≤0.8, 0≤e≤0.7, 0≤f≤0.7, and 0≤g≤0.3; and
even more preferably 0.03≤a≤0.5, 0.3≤c≤1.0, 0≤d≤0.75, 0≤e≤0.6, 0≤f≤0.6, and 0≤g≤0.2. Notably, a+c+d+e+f+g=1.0.

The polymer may be synthesized by any desired methods, for example, by dissolving one or more monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, and dioxane. Examples of the polymerization initiator used herein include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. Preferably, the reaction temperature is 50 to 80° C. and the reaction time is 2 to 100 hours, more preferably 5 to 20 hours. Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The polymer should preferably have a weight average molecular weight (Mw) in the range of 1,000 to 500,000, and more preferably 2,000 to 30,000, as measured by GPC versus polystyrene standards using tetrahydrofuran (THF) solvent. A Mw in the range ensures that a resist film has satisfactory heat resistance.

If a polymer has a wide molecular weight distribution or dispersity (Mw/Mn), which indicates the presence of lower and higher molecular weight polymer fractions, there is a possibility that foreign matter is left on the pattern or the pattern profile is degraded. The influences of Mw and Mw/Mn become stronger as the pattern rule becomes finer. Therefore, the polymer should preferably have a narrow dispersity (Mw/Mn) of 1.0 to 2.0, especially 1.0 to 1.5, in order to provide a resist composition suitable for micropatterning to a small feature size.

It is understood that a blend of two or more polymers which differ in compositional ratio, Mw or Mw/Mn is acceptable.

Organic Solvent

The resist composition also contains an organic solvent. Examples of the organic solvent are described in JP-A 2008-111103, paragraphs [0144]-[0145] (U.S. Pat. No. 7,537,880). Exemplary solvents include ketones such as cyclohexanone, cyclopentanone, and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone. Where an acid labile group of acetal type is used, a high-boiling alcohol solvent may be added for accelerating deprotection reaction of acetal, for example, diethylene glycol, propylene glycol, glycerol, 1,4-butanediol, or 1,3-butanediol.

The organic solvent is preferably added in an amount of 100 to 10,000 parts, and more preferably 200 to 8,000 parts by weight per 80 parts by weight of the base polymer.

Acid Generator

The resist composition may further comprise an acid generator capable of generating a strong acid, referred to as acid generator of addition type, hereinafter. As used herein, the term “strong acid” refers to a compound having a sufficient acidity to induce deprotection reaction of an acid labile group on the base polymer.

The acid generator is typically a compound (PAG) capable of generating an acid upon exposure to actinic ray or radiation. Although the PAG used herein may be any compound capable of generating an acid upon exposure to high-energy radiation, those compounds capable of generating sulfonic acid, imide acid (imidic acid) or methide acid are preferred. Suitable PAGs include sulfonium salts, iodonium salts, sulfonyldiazomethane, N-sulfonyloxyimide, and oxime-O-sulfonate acid generators. Exemplary PAGs are described in JP-A 2008-111103, paragraphs [0122]-[0142] (U.S. Pat. No. 7,537,880).

Sulfonium salts having the formula (2) are also useful as the PAG.

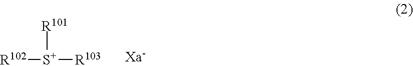

In formula (2), R¹⁰¹, R¹⁰² and R¹⁰³ are each independently a C₁-C₂₀ hydrocarbyl group which may contain a heteroatom. Any two of R¹⁰¹, R¹⁰² and R¹⁰³ may bond together to form a ring with the sulfur atom to which they are attached.

In formula (2). Xa⁻ is an anion selected from the formulae (2A) to (2D).

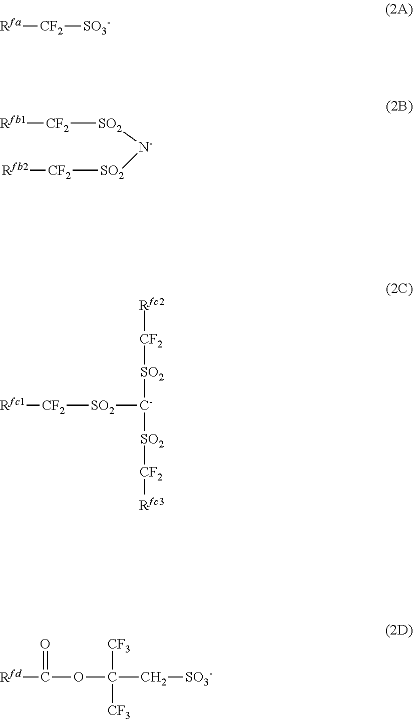

In formula (2A). R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified later for R¹¹¹ in formula (2A′).

Of the anions of formula (2A), a structure having the formula (2A′) is preferred.

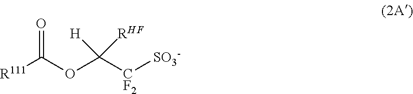

In formula (2A′), R^{HF} is hydrogen or trifluoromethyl, preferably trifluoromethyl.

R¹¹¹ is a C₁-C₃₀ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation. The hydrocarbyl group R¹¹¹ may be saturated or unsaturated and straight, branched or cyclic. Suitable hydrocarbyl groups include C₁-C₃₀ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, icosanyl; C₃-C₃₀ cyclic saturated hydrocarbyl groups such as cyclopentyl, cyclohexyl, 1-adamantyl, 2-adamantyl, 1-adamantylmethyl, norbornyl, norborylmethyl, tricyclodecanyl, tetracyclododecanyl, tetracyclododecanylmethyl, dicyclohexylmethyl; C₂-C₃₀ unsaturated aliphatic hydrocarbyl groups such as allyl and 3-cyclohexenyl; C₆-C₃₀ aryl groups such as phenyl, 1-naphthyl, 2-naphthyl: C₇-C₃₀ aralkyl groups such as benzyl and diphenylmethyl, and combinations thereof.

In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

With respect to the synthesis of the sulfonium salt having an anion of formula (2A′), reference is made to JP-A 2007-145797, JP-A 2008-106045, JP-A 2009-007327, and JP-A 2009-258695. Also useful are the sulfonium salts described in JP-A 2010-215608, JP-A 2012-041320, JP-A 2012-106986, and JP-A 2012-153644.

Examples of the anion having formula (2A) are shown below, but not limited thereto.

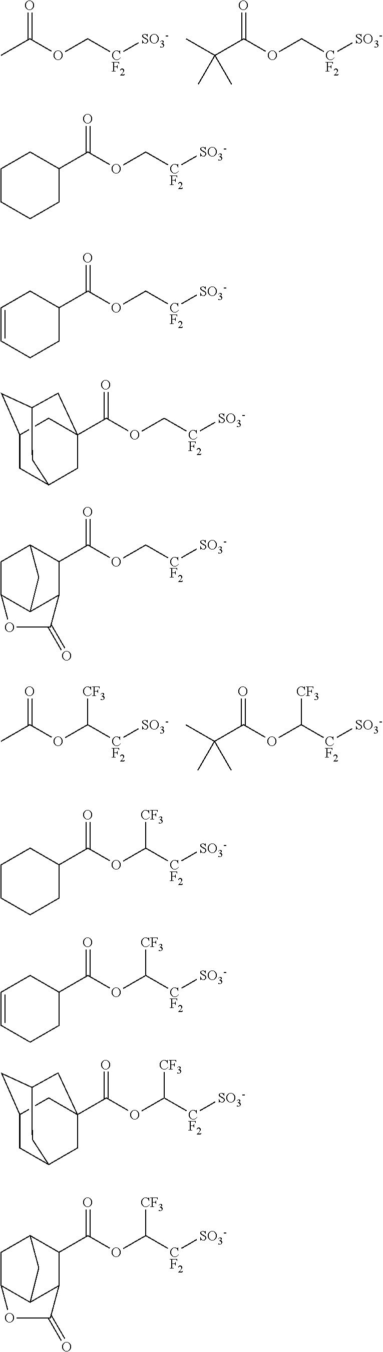

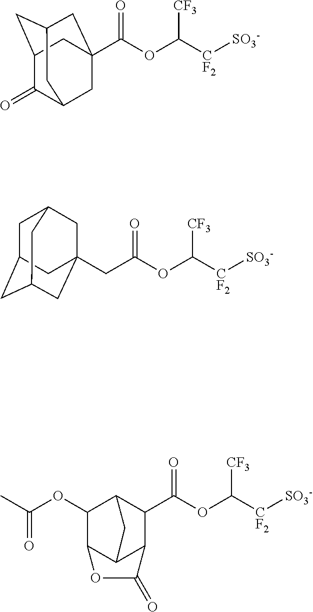

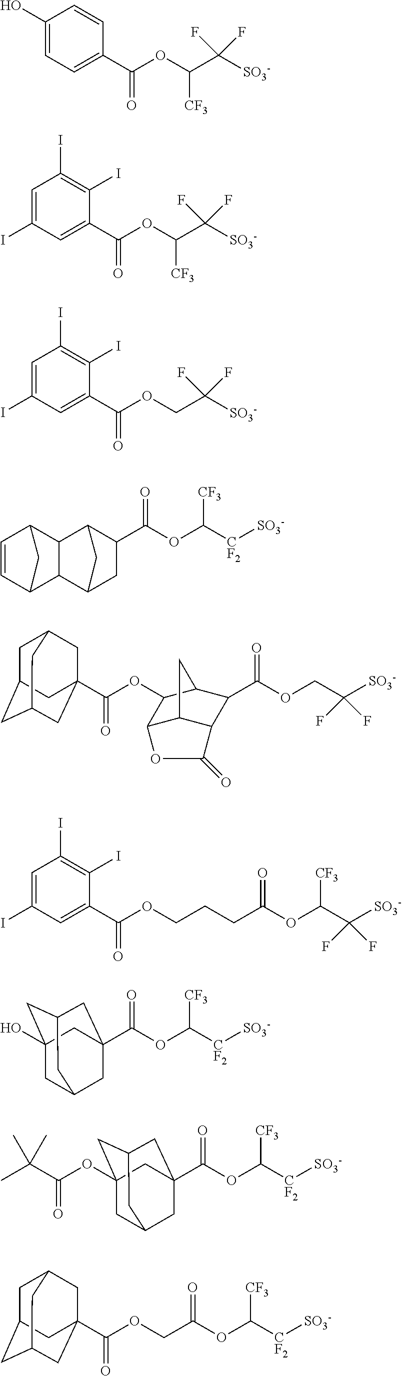

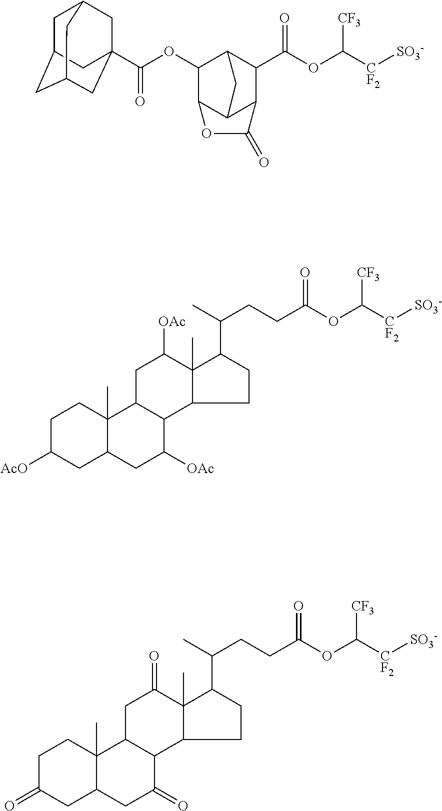

In formula (2B), R^{fb1} and R^{fb2} are each independently fluorine or a C₁-C₄₀ hydrocarbyl group which may contain a heteroatom. Suitable hydrocarbyl groups are as exemplified above for R¹¹¹ in formula (1A′). Preferably R^{fb1} and R^{fb2} each are fluorine or a straight C₁-C₄ fluorinated alkyl group. A pair of R^{fb1} and R^{fb2} may bond together to form a ring with the linkage (—CF₂—SO₂—N⁻—SO₂—CF₂—) to which they are attached, and the ring-forming pair is preferably a fluorinated ethylene or fluorinated propylene group.

In formula (2C), R^{fc1}, R^{fc2} and R^{fc3} are each independently fluorine or a C₁-C₄₀ hydrocarbyl group which may contain a heteroatom. Suitable hydrocarbyl groups are as exemplified above for R¹¹¹ in formula (1A′). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C⁻—SO₂—CF₂—) to which they are attached, and the ring-forming pair is preferably a fluorinated ethylene or fluorinated propylene group.

In formula (2D), R^{fd} is a C₁-C₄₀ hydrocarbyl group which may contain a heteroatom. The hydrocarbyl group may be saturated or unsaturated and straight, branched or cyclic. Suitable hydrocarbyl groups are as exemplified above for R¹¹¹.

With respect to the synthesis of the sulfonium salt having an anion of formula (2D), reference is made to JP-A 2010-215608.

Examples of the anion having formula (2D) are shown below, but not limited thereto.

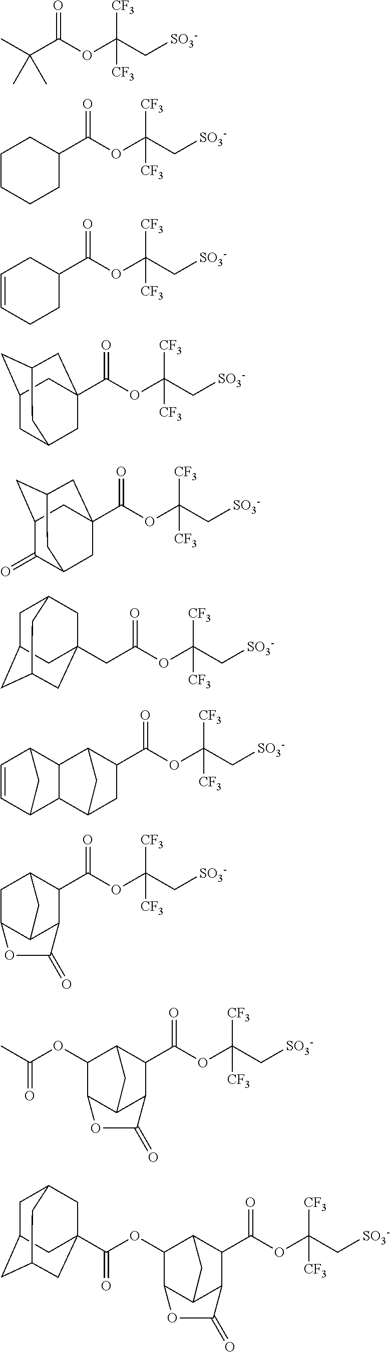

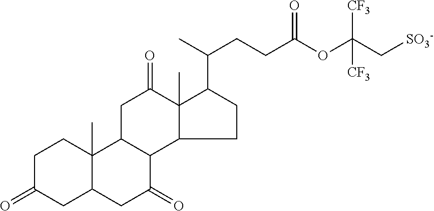

The compound having the anion of formula (2D) has a sufficient acid strength to cleave acid labile groups in the base polymer because it is free of fluorine at α-position of sulfo group, but has two trifluoromethyl groups at β-position. Thus the compound is a useful PAG.

Of the foregoing PAGs, those having an anion of formula (2A′) or (2D) are especially preferred because of reduced acid diffusion and high solubility in the resist solvent.

In the resist composition, the acid generator of addition type is typically used in an amount of 0 to 200 parts, preferably 0.1 to 100 parts by weight per 80 parts by weight of the base polymer. The acid generator of addition type may be used alone or in admixture.

Quencher

The resist composition may further contain a quencher. As used herein, the quencher refers to a compound capable of trapping the acid, which is generated by the acid generator in the resist composition upon light exposure, to prevent the acid from diffusing to the unexposed region.

The quencher is typically selected from conventional basic compounds. Conventional basic compounds include primary, secondary, and tertiary aliphatic amines, mixed amines, aromatic amines, heterocyclic amines, nitrogen-containing compounds with carboxy group, nitrogen-containing compounds with sulfonyl group, nitrogen-containing compounds with hydroxy group, nitrogen-containing compounds with hydroxyphenyl group, alcoholic nitrogen-containing compounds, amide derivatives, imide derivatives, and carbamate derivatives. Also included are primary, secondary, and tertiary amine compounds, specifically amine compounds having a hydroxy group, ether bond, ester bond, lactone ring, cyano group, or sulfonic ester bond as described in JP-A 2008-111103, paragraphs [0146]-[0164], and compounds having a carbamate group as described in JP 3790649. Addition of a basic compound may be effective for further suppressing the diffusion rate of acid in the resist film or correcting the pattern profile.

Onium salts such as sulfonium salts, iodonium salts and ammonium salts of sulfonic acids which are not fluorinated at α-position may also be used as the quencher. While an α-fluorinated sulfonic acid, imide acid, and methide acid are necessary to deprotect the acid labile group of carboxylic acid ester, an α-non-fluorinated sulfonic acid or carboxylic acid is released by salt exchange with an α-non-fluorinated onium salt. An α-non-fluorinated sulfonic acid and a carboxylic acid function as a quencher because they do not induce deprotection reaction.

Also, onium salts of carboxylic acid having the formula (3) are useful quenchers.

R²⁰¹—CO₂⁻Mq⁺ (3)

In formula (3), R²⁰¹ is a C₁-C₄₀ hydrocarbyl group which may contain a heteroatom. The hydrocarbyl group may be saturated or unsaturated and straight, branched or cyclic. Mq⁺ is an onium cation. Suitable onium cations include sulfonium, iodonium and ammonium cations.

In the onium salt of carboxylic acid, an anion having the formula (3A) is preferred.

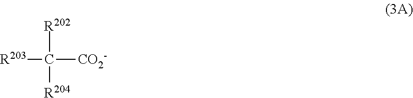

Herein R²⁰² and R²⁰³ are each independently hydrogen, fluorine, or trifluoromethyl. R²⁰⁴ is hydrogen, hydroxy, or a C₁-C₃₅ hydrocarbyl group which may contain a heteroatom.

Also useful are quenchers of polymer type as described in U.S. Pat. No. 7,598,016 (JP-A 2008-239918). The polymeric quencher segregates at the resist film surface after coating and thus enhances the rectangularity of resist pattern. When a protective film is applied as is often the case in the immersion lithography, the polymeric quencher is also effective for preventing a film thickness loss of resist pattern or rounding of pattern top.

When the resist composition contains a quencher, the quencher is preferably added in an amount of 0 to 5 parts by weight, more preferably 0 to 4 parts by weight per 80 parts by weight of the base polymer. The quencher may be used alone or in admixture.

Surfactant

The resist composition may further include a surfactant which is insoluble or substantially insoluble in water and soluble in alkaline developer, and/or a surfactant which is insoluble or substantially insoluble in water and alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

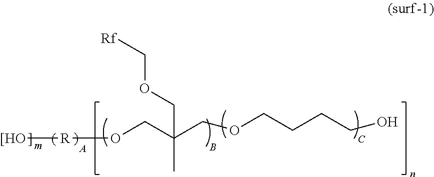

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

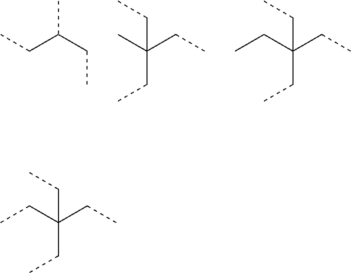

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (4A) to (4E).

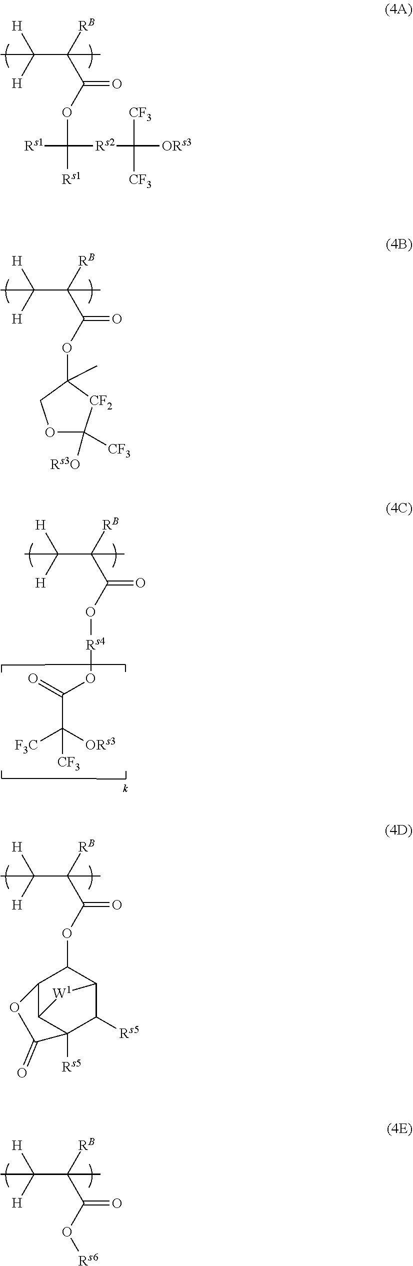

Herein, R^{B} is hydrogen, fluorine, methyl or trifluoromethyl. W¹ is —CH₂—, —CH₂CH₂— or —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (k+1)-valent hydrocarbon or fluorinated hydrocarbon group, and k is an integer of 1 to 3. R^{s5} is each independently hydrogen or a group: —C(═O)—O—R^{s7} wherein R^{s7} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group represented by R^{s1} may be straight, branched or cyclic. Examples thereof include methyl, ethyl, n-propyl, isopropyl, cyclopropyl, n-butyl, isobutyl, sec-butyl, tert-butyl, cyclobutyl, n-pentyl, cyclopentyl, n-hexyl, cyclohexyl, n-heptyl, n-octyl, n-nonyl, n-decyl, adamantyl, and norbornyl. Inter alia, C₁-C₆ hydrocarbyl groups are preferred.

The hydrocarbylene group represented by R^{s2} may be straight, branched or cyclic. Examples thereof include methylene, ethylene, propylene, butylene and pentylene.

The hydrocarbyl group represented by R^{s3} or R^{s6} may be straight, branched or cyclic. Examples thereof include alkyl, alkenyl and alkynyl groups, with the alkyl groups being preferred. Suitable alkyl groups include those exemplified for the hydrocarbyl group represented by R^{s1} as well as n-undecyl, n-dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-1) to (AL-3), C₄-C₂₀, preferably C₄-C₁₅ tertiary hydrocarbyl groups, trialkylsilyl groups in which each alkyl moiety has 1 to 6 carbon atoms, and C₄-C₂₀ oxoalkyl groups.

The (k+1)-valent hydrocarbon or fluorinated hydrocarbon group represented by R^{s4} may be straight, branched or cyclic and examples thereof include the foregoing hydrocarbyl or fluorinated hydrocarbyl groups from which k number of hydrogen atoms are eliminated.

The fluorinated hydrocarbyl group represented by R^{s7} may be straight, branched or cyclic. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (4A) to (4E) are shown below, but not limited thereto. Herein R^{B} is as defined above.

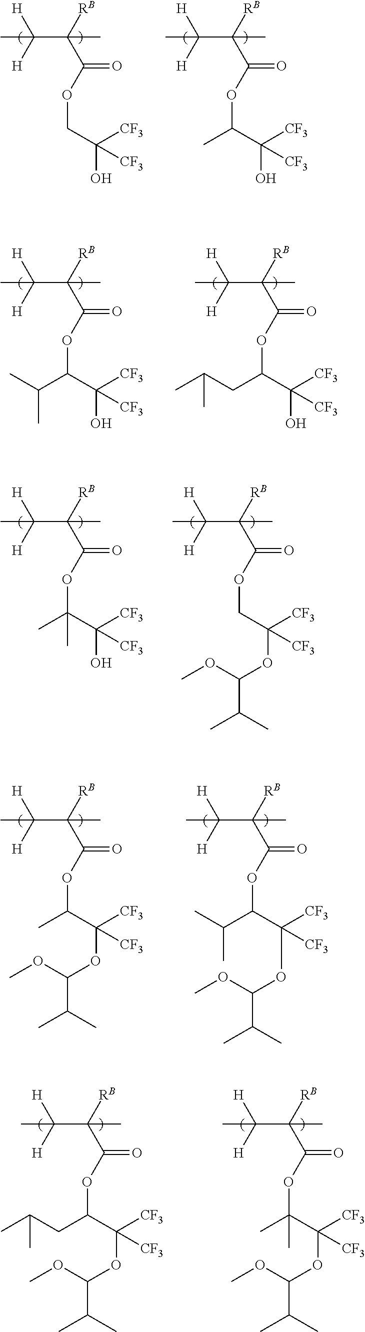

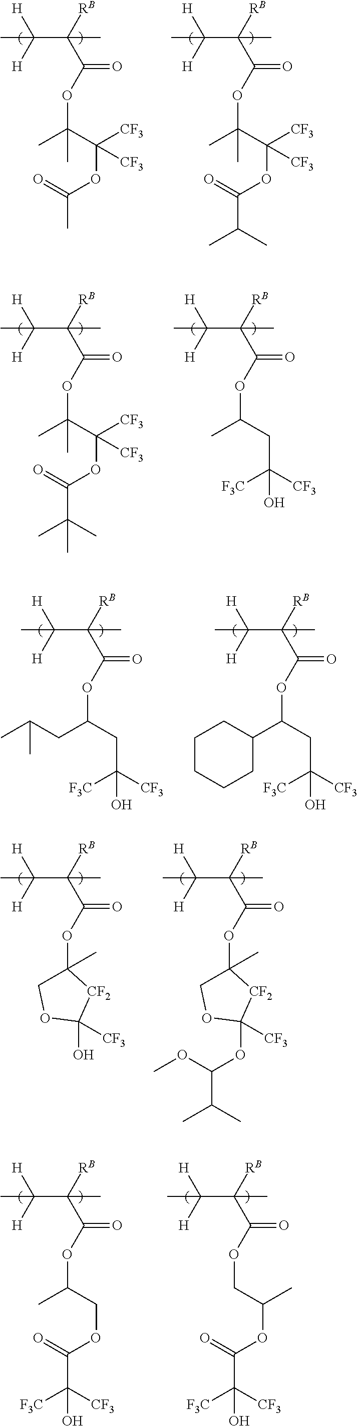

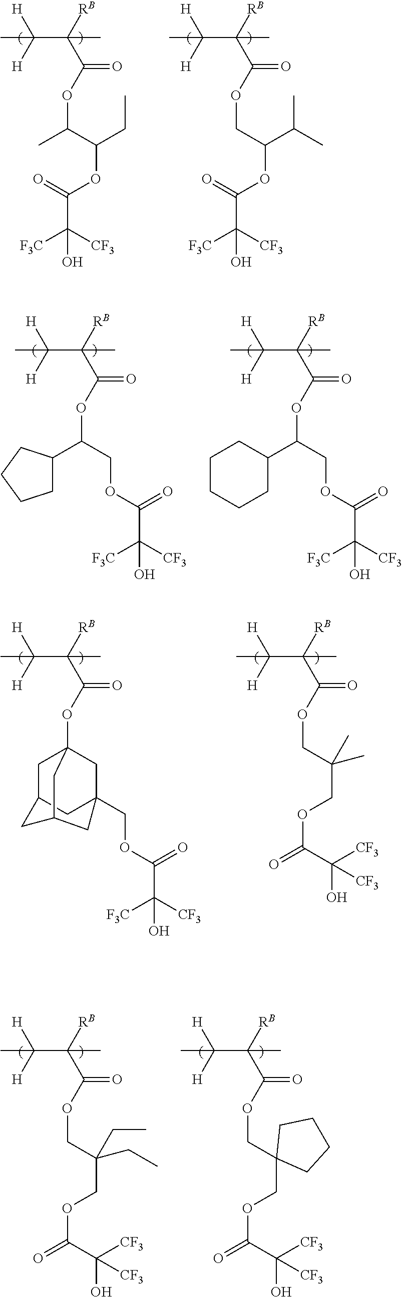

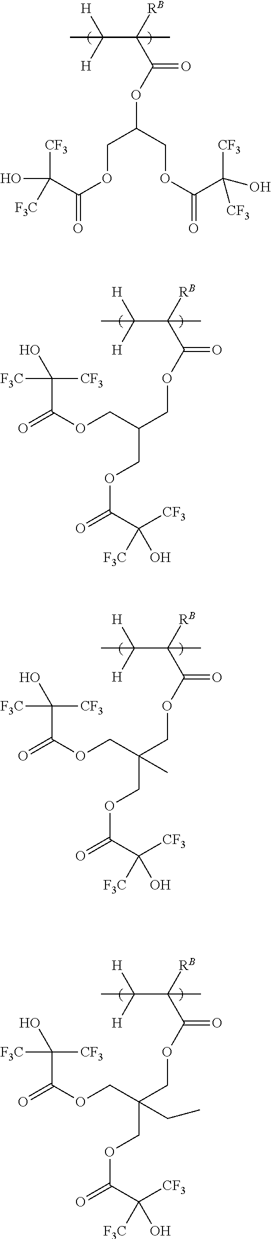

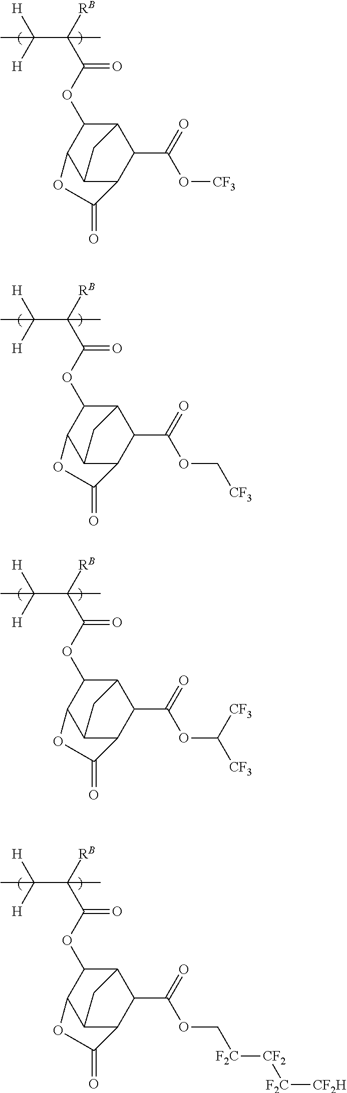

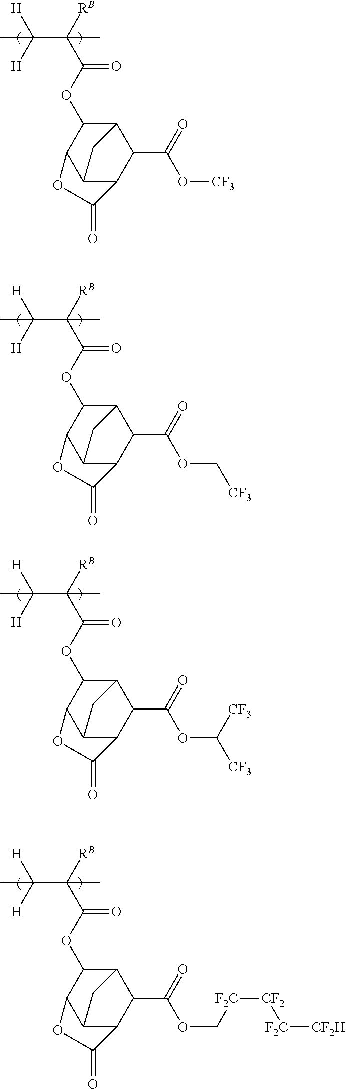

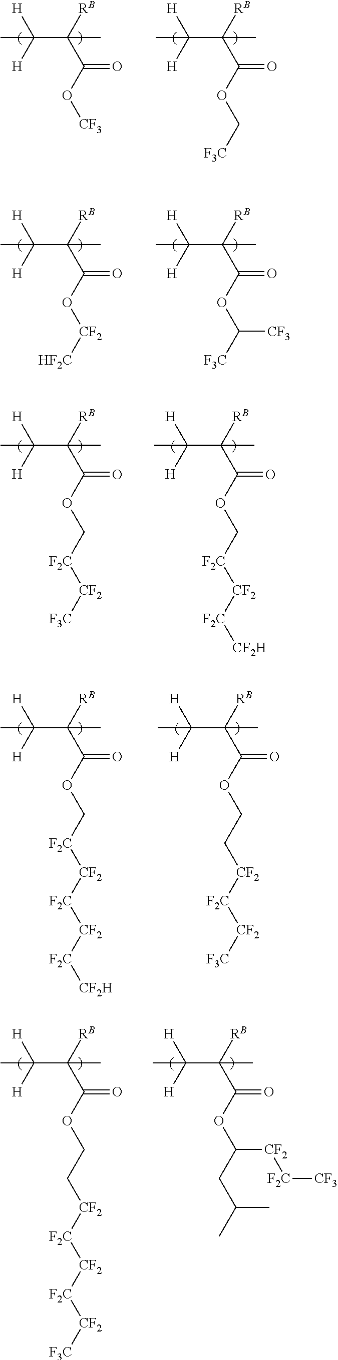

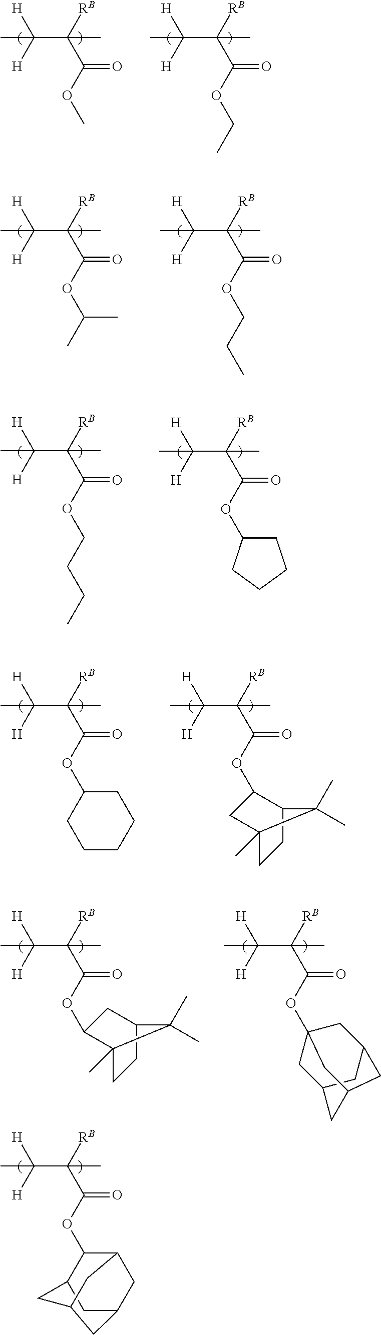

The polymeric surfactant may further contain repeat units other than the repeat units having formulae (4A) to (4E). Typical other repeat units are those derived from methacrylic acid and α-trifluoromethylacrylic acid derivatives. In the polymeric surfactant, the content of repeat units having formulae (4A) to (4E) is preferably at least 20 mol %, more preferably at least 60 mol %, most preferably 100 mol % of the overall repeat units.

The polymeric surfactant preferably has a Mw of 1,000 to 500,000, more preferably 3,000 to 100,000 and a Mw/Mn of 1.0 to 2.0, more preferably 1.0 to 1.6.

The polymeric surfactant may be synthesized by any desired method, for example, by dissolving an unsaturated bond-containing monomer or monomers providing repeat units having formula (4A) to (4E) and optionally other repeat units in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

In the resist composition, the surfactant is preferably used in an amount of 0 to 20 parts by weight per 80 parts by weight of the base polymer. When the surfactant is added, its amount is preferably at least 0.001 part by weight, more preferably at least 0.01 part by weight and preferably up to 15 parts by weight, more preferably up to 10 parts by weight. The surfactant may be used alone or in admixture.

Other Components

The resist composition may further comprise other components, for example, a compound which is decomposed with an acid to generate another acid (i.e., acid amplifier compound), organic acid derivative, fluorinated alcohol, dissolution inhibitor, crosslinker, and acetylene alcohol. Each of the other components may be used alone or in admixture.

The acid amplifier compound is described in JP-A 2009-269953 and JP-A 2010-215608. The acid amplifier compound is preferably used in an amount of 0 to 5 parts, more preferably 0 to 3 parts by weight per 80 parts by weight of the base polymer. An extra amount of the acid amplifier compound can make the acid diffusion control difficult and cause degradations to resolution and pattern profile. With respect to the organic acid derivative and fluorinated alcohol, reference should be made to JP-A 2009-269953 and JP-A 2010-215608.

Where the resist composition is of positive tone, a dissolution inhibitor is blended to further increase the difference in dissolution rate between exposed and unexposed regions for thereby further improving the resolution. The dissolution inhibitor is a compound adapted to change solubility in developer under the action of acid and having a Mw of up to 3,000. Specifically, it is a compound having at least two phenolic hydroxyl groups on the molecule, in which an average of from 0 to 100 mol % of all the hydrogen atoms on the phenolic hydroxyl groups are replaced by acid labile groups or a compound having at least one carboxyl group on the molecule, in which an average of 50 to 100 mol % of all the hydrogen atoms on the carboxyl groups are replaced by acid labile groups, both the compounds having a Mw of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is replaced by an acid labile group, as described in US 2008090172 (JP-A 2008-122932, paragraphs [0155] to [0178]).

Where the resist composition is of positive tone and contains a dissolution inhibitor, the inhibitor is preferably used in an amount of 0 to 50 parts by weight, more preferably 5 to 40 parts by weight per 80 parts by weight of the base polymer.

Where the resist composition is of negative tone, a negative pattern may be formed by adding a crosslinker to reduce the dissolution rate of a resist film in exposed area. Suitable crosslinkers which can be used herein include epoxy compounds, melamine compounds, guanamine compounds, glycoluril compounds and urea compounds having substituted thereon at least one group selected from among methylol, alkoxymethyl and acyloxymethyl groups, isocyanate compounds, azide compounds, and compounds having a double bond such as an alkenyloxy group. These compounds may be used as an additive or introduced into a polymer side chain as a pendant. Hydroxy-containing compounds may also be used as the crosslinker.

Of the foregoing crosslinkers, examples of the epoxy compound include tris(2,3-epoxypropyl) isocyanurate, trimethylolmethane triglycidyl ether, trimethylolpropane triglycidyl ether, and triethylolethane triglycidyl ether. Examples of the melamine compound include hexamethylol melamine, hexamethoxymethyl melamine, hexamethylol melamine compounds having 1 to 6 methylol groups methoxymethylated and mixtures thereof, hexamethoxyethyl melanine, hexaacyloxymethyl melamine, hexamethylol melamine compounds having 1 to 6 methylol groups acyloxymethylated and mixtures thereof. Examples of the guanamine compound include tetramethylol guanamine, tetramethoxymethyl guanamine, tetramethylol guanamine compounds having 1 to 4 methylol groups methoxymethylated and mixtures thereof, tetramethoxyethyl guanamine, tetraacyloxyguanamine, tetramethylol guanamine compounds having 1 to 4 methylol groups acyloxymethylated and mixtures thereof. Examples of the glycoluril compound include tetramethylol glycoluril, tetramethoxyglycoluril, tetramethoxymethyl glycoluril, tetramethylol glycoluril compounds having 1 to 4 methylol groups methoxymethylated and mixtures thereof, tetramethylol glycoluril compounds having 1 to 4 methylol groups acyloxymethylated and mixtures thereof. Examples of the urea compound include tetramethylol urea, tetramethoxymethylurea, tetramethylol urea compounds having 1 to 4 methylol groups methoxymethylated and mixtures thereof, and tetramethoxyethyl urea.

Suitable isocyanate compounds include tolylene diisocyanate, diphenylmethane diisocyanate, hexamethylene diisocyanate and cyclohexane diisocyanate. Suitable azide compounds include 1,1′-biphenyl-4,4′-bisazide, 4,4′-methylidenebisazide, and 4,4′-oxybisazide. Examples of the alkenyloxy group-containing compound include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylol propane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylol propane trivinyl ether.

Where the resist composition is of negative tone and contains a crosslinker, the crosslinker is preferably added in an amount of 0.1 to 50 parts, more preferably 1 to 40 parts by weight per 80 parts by weight of the base polymer.

Suitable acetylene alcohols are described in JP-A 2008-122932, paragraphs [0179]-[0182]. An appropriate amount of the acetylene alcohol blended is 0 to 5 parts by weight per 80 parts by weight of the base polymer.

Process

The resist composition is used in the fabrication of various integrated circuits. Pattern formation using the resist composition may be performed by well-known lithography processes. The process generally involves the steps of applying the resist composition onto a substrate to form a resist film thereon, exposing the resist film to high-energy radiation, and developing the exposed resist film in a developer. If necessary, any additional steps may be added.

For example, the resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The coating is prebaked on a hotplate at a temperature of 60 to 150° C. for 10 seconds to 30 minutes, preferably at 80 to 120° C. for 30 seconds to 20 minutes. The resulting resist film is generally 0.01 to 2 μm thick.

Then the resist film is exposed to high-energy radiation. Examples of the high-energy radiation include UV, deep-UW, EB, EUV of wavelength 3 to 15 nm, x-ray, soft x-ray, excimer laser light, γ-ray or synchrotron radiation. On use of UV, deep UV, EUV, x-ray, soft x-ray, excimer laser, γ-ray or synchrotron radiation, the resist film is exposed directly or through a mask having a desired pattern, preferably in a dose of about 1 to 200 mJ/cm², more preferably about 10 to 100 mJ/cm². On use of EB, a pattern may be written directly or through a mask having a desired pattern, preferably in a dose of about 0.1 to 100 μC/cm², more preferably about 0.5 to 50 μC/cm². The resist composition is suited for micropatterning using high-energy radiation such as KrF excimer laser, ArF excimer laser, EB, EUV, x-ray, soft x-ray, γ-ray or synchrotron radiation.

After the exposure, the resist film may be baked (PEB) on a hotplate at 60 to 150° C. for 10 seconds to 30 minutes, preferably at 80 to 120° C. for 30 seconds to 20 minutes.

After the exposure or PEB, the resist film is developed with a developer in the form of an aqueous base solution for 3 seconds to 3 minutes, preferably 5 seconds to 2 minutes by conventional techniques such as dip, puddle and spray techniques. A typical developer is a 0.1 to 10 wt %, preferably 2 to 5 wt % aqueous solution of tetramethylammonium hydroxide (TMAH), tetraethylammonium hydroxide (TEAH), tetrapropylammonium hydroxide (TPAH), or tetrabutylammonium hydroxide (TBAH). The resist film in the exposed area is dissolved in the developer whereas the resist film in the unexposed area is not dissolved. In this way, the desired positive pattern is formed on the substrate. Inversely in the case of negative resist, the exposed area of resist film is insolubilized and the unexposed area is dissolved in the developer.

In an alternative embodiment, a negative pattern may be formed via organic solvent development using a positive resist composition comprising a base polymer having an acid labile group. The developer used herein is preferably selected from among 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate, and mixtures thereof.

At the end of development, the resist film is rinsed. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents. Specifically, suitable alcohols of 3 to 10 carbon atoms include n-propyl alcohol, isopropyl alcohol, 1-butyl alcohol, 2-butyl alcohol, isobutyl alcohol, t-butyl alcohol, 1-pentanol, 2-pentanol, 3-pentanol, t-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol. Suitable ether compounds of 8 to 12 carbon atoms include di-n-butyl ether, diisobutyl ether, di-s-butyl ether, di-n-pentyl ether, diisopentyl ether, di-s-pentyl ether, di-t-pentyl ether, and di-n-hexyl ether. Suitable alkanes of 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Suitable alkenes of 6 to 12 carbon atoms include hexene, heptene, octene, cyclohexene, methylcyclohexene, dimethylcyclohexene, cycloheptene, and cyclooctene. Suitable alkynes of 6 to 12 carbon atoms include hexyne, heptyne, and octyne. Suitable aromatic solvents include toluene, xylene, ethylbenzene, isopropylbenzene, t-butylbenzene and mesitylene. The solvents may be used alone or in admixture.

Rinsing is effective for minimizing the risks of resist pattern collapse and defect formation. However, rinsing is not essential. If rinsing is omitted, the amount of solvent used may be reduced.

A hole or trench pattern after development may be shrunk by the thermal flow, RELACS® or DSA process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

### EXAMPLES

Examples of the invention are given below by way of illustration and not by way of limitation. The abbreviation “pbw” is parts by weight. Analysis is made by IR spectroscopy, NMR spectroscopy, and time-of-flight mass spectrometry (TOF-MS) using analytic instruments as shown below.
IR: NICOLET 6700 by Thermo Fisher Scientific Inc.
¹H-NMR: ECA-500 by JEOL Ltd.
¹⁹F-NMR: ECA-500 by JEOL Ltd.
MALDI TOF-MS: S3000 by JEOL Ltd.

[1] Synthesis of Onium Salt Compounds

### Example 1-1: Synthesis of Onium Salt Compound PAG-1

(1) Synthesis of Compound C-4

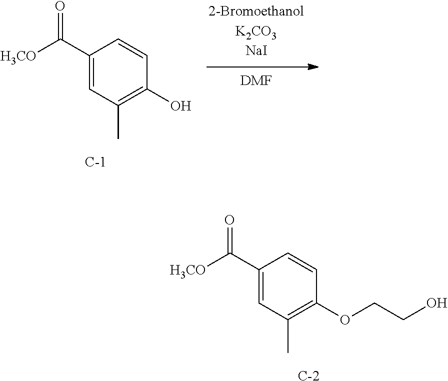

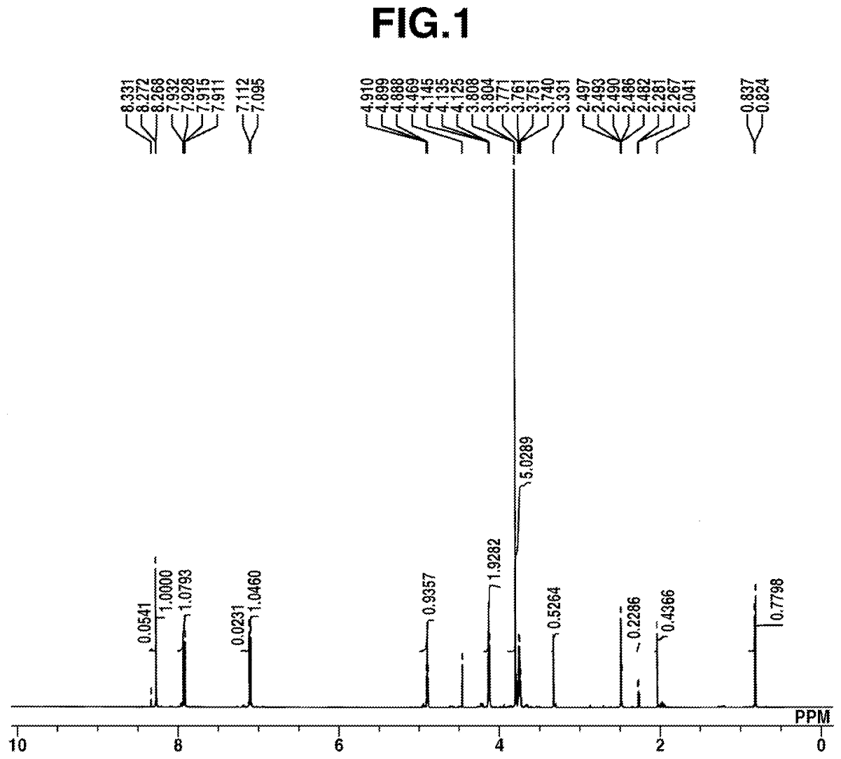

580 g of Reactant C-1, 520 g of 2-bromoethanol, 726 g of potassium carbonate, 31.5 g of sodium iodide, and 3,970 g of dimethylformamide (DMF) were mixed and stirred at 90° C. for 20 hours. At the end of stirring, the solution was ice cooled, combined with 3,600 g of deionized water, and stirred for 30 minutes. Then 4,500 g of methyl isobutyl ketone (MIBK) was added to the solution, which was stirred for 1 hour. The organic layer was taken out and subjected to ordinary aqueous work-up. After the solvent was distilled off, 3.500 g of hexane was added to the residue and stirred for 2 hours. Crystallization was followed by filtration, obtaining 604 g of Compound C-2 as solids. The ¹H-NMR (500 MHz, DMSO-d₆) spectrum of Compound C-2 is shown in FIG. 1.

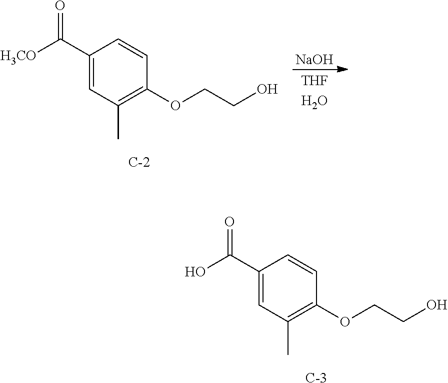

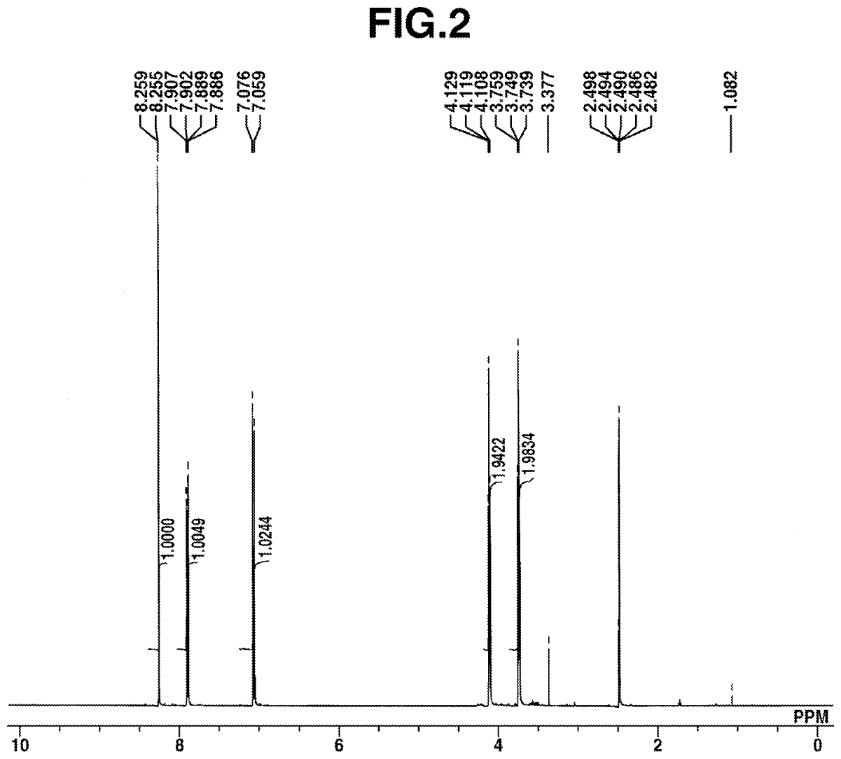

600 g of Compound C-2 was added to 2,800 g of THF, which was stirred under ice cooling. To the solution, 330 g of 25 wt % sodium hydroxide aqueous solution and 900 g of deionized water were added dropwise. The solution was stirred at 40° C. for 16 hours. At the end of reaction, the organic solvent was distilled off. The aqueous solution was washed with tert-butyl methyl ether (TBME). 430 g of 20 wt % hydrochloric acid and 1 L of hexane were added to the solution, which was stirred. Crystallization was followed by filtration, obtaining 560 g of Compound C-3 as solids. The ¹H-NMR (5001 MHz, DMSO-d₆) spectrum of Compound C-3 is shown in FIG. 2.

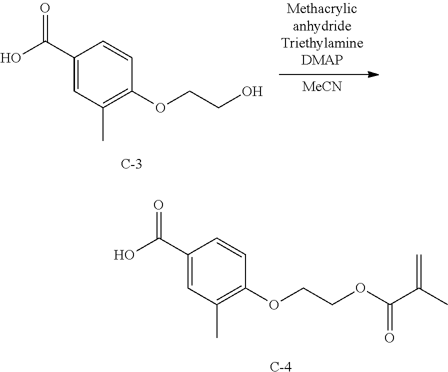

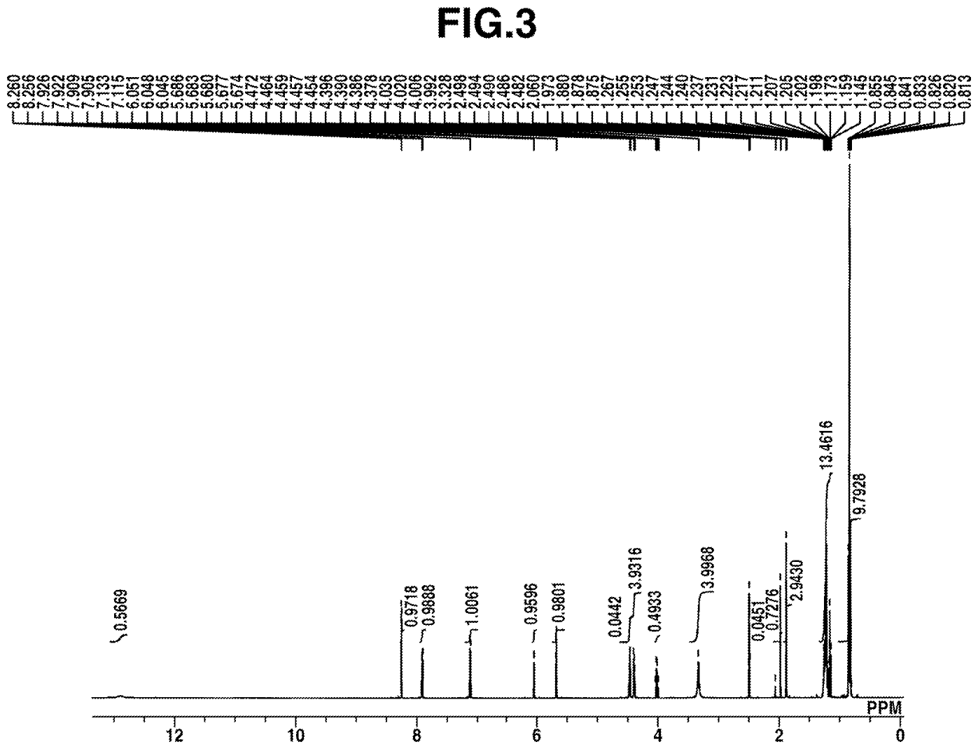

560 g of Compound C-3, 340 g of methacrylic anhydride, 3,000 g of acetonitrile, and an amount (1000 ppm/theoretical yield) of a polymerization inhibitor were mixed to form a solution. Under ice cooling, a mixture of 442 g of triethylamine, 22 g of 4-dimethylaminopyridine (DMAP) and 600 g of acetonitrile was added dropwise to the solution. The mixture was stirred for 3 hours under ice cooling. At the end of reaction, 1,900 g of 5 wt % sodium bicarbonate aqueous solution was added to the solution under ice cooling, followed by 20 minutes of stirring. 1,070 g of 20 wt % hydrochloric acid and 5,300 g of deionized water were added to the solution and stirred for crystallization. The precipitate was collected by filtration and dissolved in 4,500 g of ethyl acetate. The organic layer was taken out, washed with saturated brine and deionized water, and treated with active carbon. After the solvent was distilled off, 5.7 L of hexane was added and stirred. Crystallization was followed by filtration, obtaining 460 g of Compound C-4 as solids. The ¹H-NMR (500 MHz, DMSO-d₆) spectrum of Compound C-4 is shown in FIG. 3.

(2) Synthesis of Acid Chloride Compound C-5

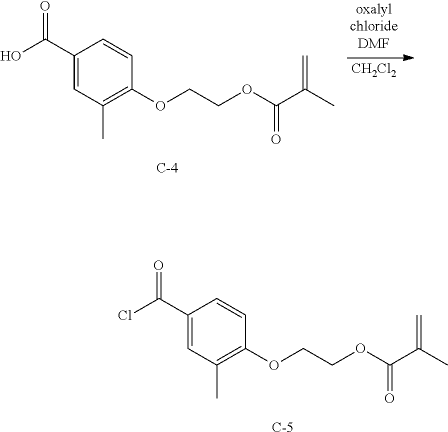

460 g of Compound C-4, 2.6 g of DMF, 2,800 g of methylene chloride, and an amount (1000 ppm/theoretical yield) of a polymerization inhibitor were mixed to form a solution, to which 186 g of oxalyl chloride was added dropwise at room temperature. Stirring was performed at room temperature for 3 hours. At the end of stirring, the solvent was distilled off, obtaining 473 g of acid chloride compound C-5.

(3 Synthesis of Onium Salt Compound PAG-1

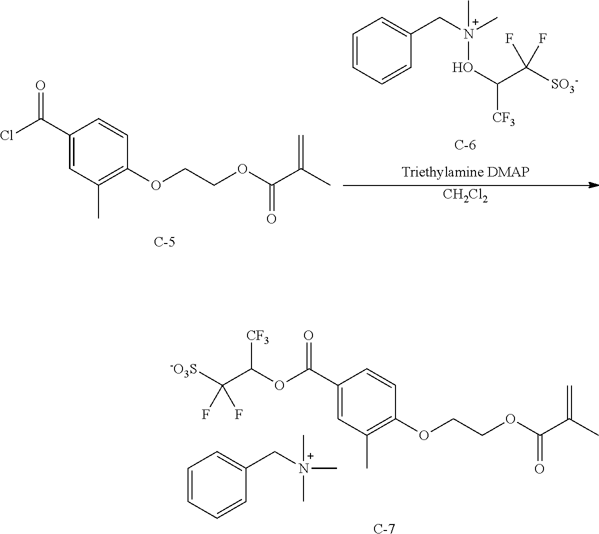

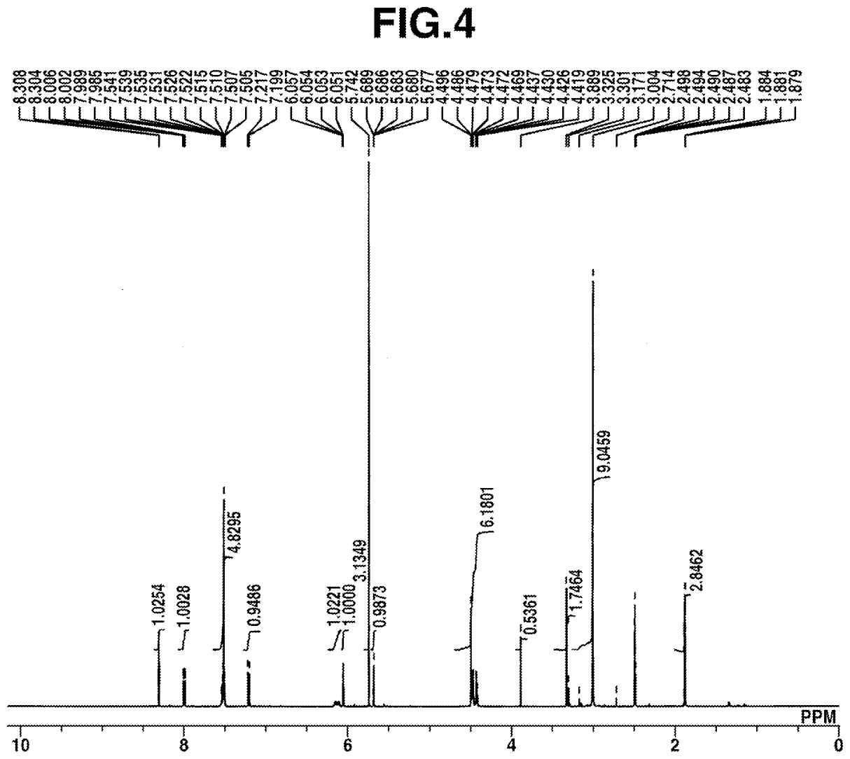

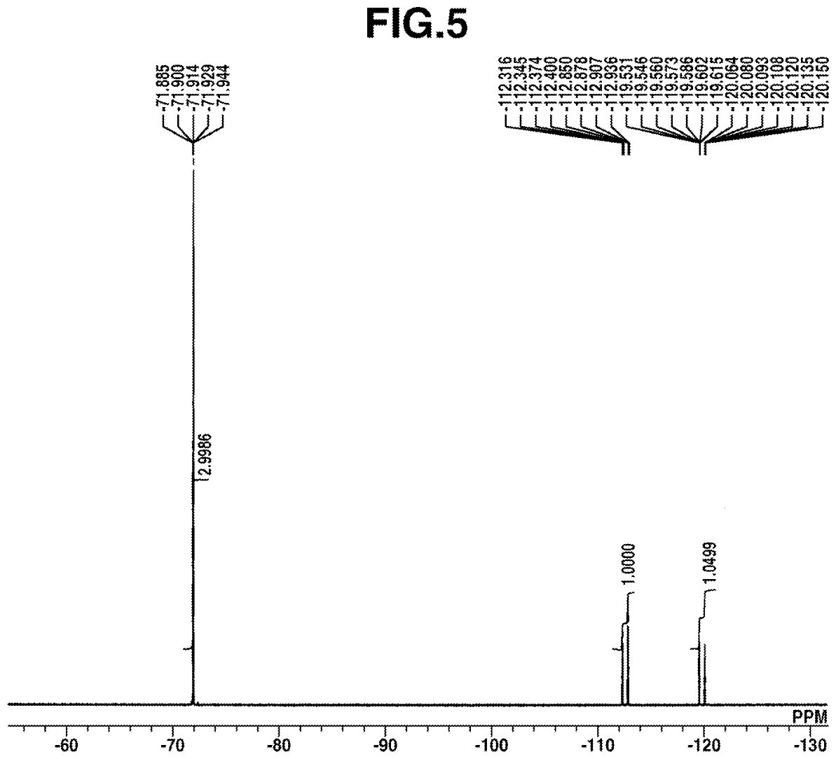

570 g of Compound C-6, 186 g of triethylamine, 15 g of DMAP, 2,000 g of methylene chloride, and an amount (1000 ppm/theoretical yield) of a polymerization inhibitor were mixed to form a solution. Under ice cooling, 473 g of Compound C-5 in 500 g of methylene chloride was added dropwise to the solution. Stirring was performed at room temperature for 20 hours. At the end of reaction, 1,000 g of 5 wt % hydrochloric acid aqueous solution was added under ice cooling, followed by 30 minutes of stirring. The organic layer was taken out and subjected to ordinary aqueous work-up. The solvent was distilled off, obtaining 856 g of Compound C-7 as oily matter. The ¹H-NMR (500 MHz, DMSO-d₆) spectrum of Compound C-7 is shown in FIG. 4. The ¹⁹F-NMR (500 MHz, DMSO-d₆) spectrum of Compound C-7 is shown in FIG. 5.

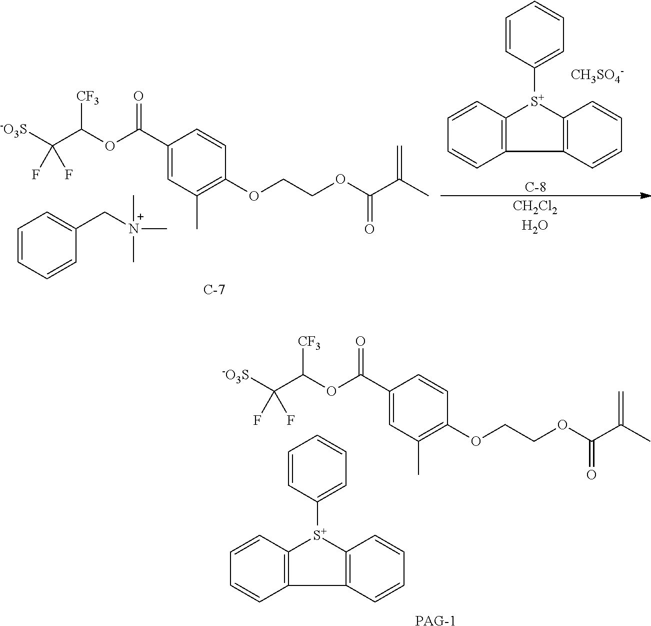

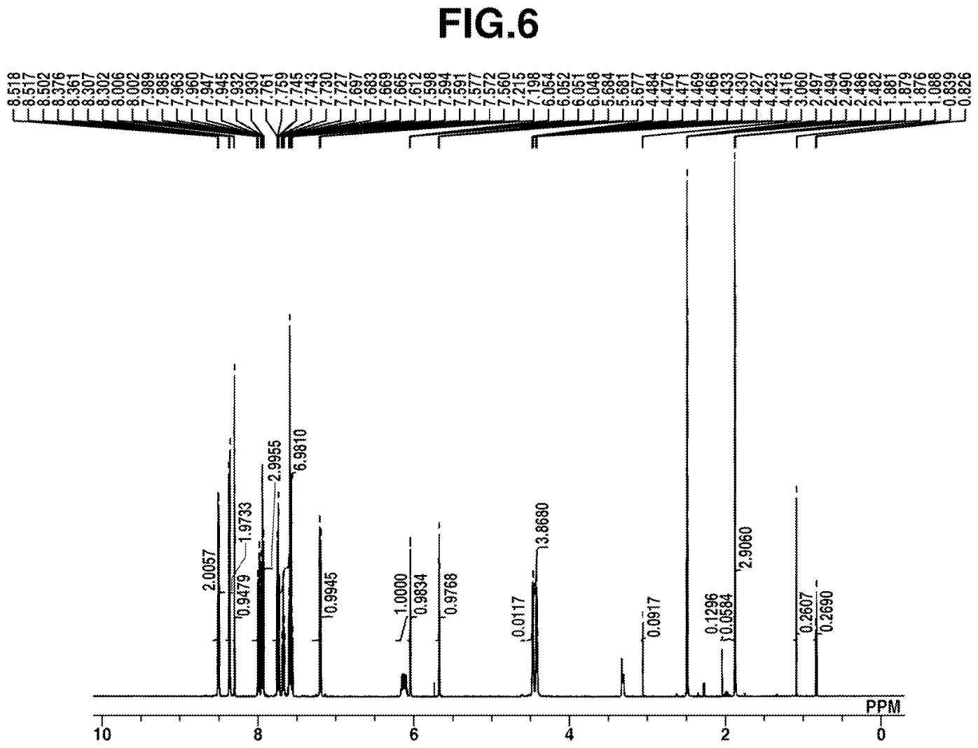

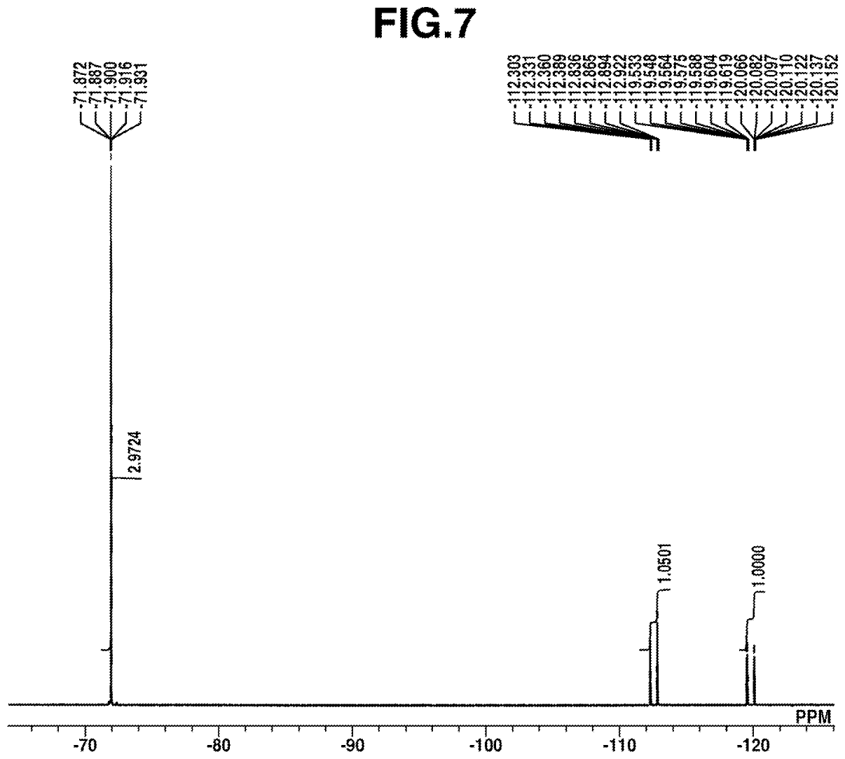

113 g of Compound C-7, 68.5 g of Compound C-8, 680 g of methylene chloride, 350 g of deionized water, and an amount (1000 ppm/theoretical yield) of a polymerization inhibitor were mixed and stirred at room temperature for 2 hours. The organic layer was taken out and subjected to ordinary aqueous work-up. After the solvent was distilled off, 140 g of MIBK was added to the residue and azeotroped to form a solution having a concentration of about 50 wt %, which was stirred at room temperature for 2 hours. 200 g of TBME was added to the solution and stirred. Crystallization was followed by filtration, obtaining 116 g of onium salt compound PAG-1 as solids. The ¹H-NMR (500 MHz, DMSO-d₆) spectrum of onium salt compound PAG-1 is shown in FIG. 6. The ¹⁹F-NMR (500 MHz, DMSO-d₆) spectrum of onium salt compound PAG-1 is shown in FIG. 7.

### Example 1-2: Synthesis of Onium Salt Compound PAG-4

(1) Synthesis of Compound C-12

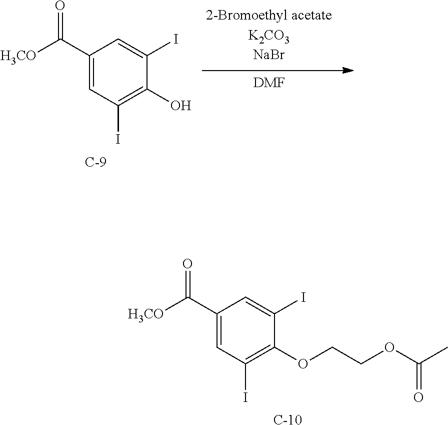

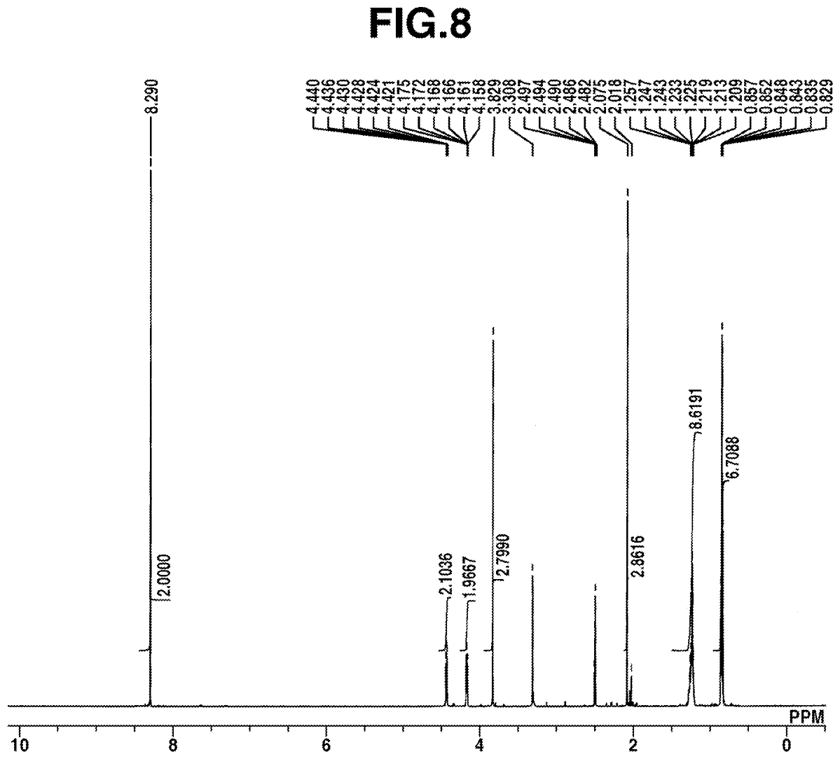

370 g of Reactant C-9, 229 g of 2-bromoethyl acetate, 189 g of potassium carbonate, 9.5 g of sodium bromide, and 2,220 g of DMF were mixed and stirred at 90° C. for 18 hours. At the end of stirring, the solution was ice cooled, combined with 2,800 g of deionized water, and stirred for 30 minutes. Then 2,800 g of MIBK was added to the solution, which was stirred for 1 hour. The organic layer was taken out and subjected to ordinary aqueous work-up. After the solvent was distilled off, 1,800 g of hexane was added to the residue and stirred for 2 hours. Crystallization was followed by filtration, obtaining 448 g of Compound C-10 as solids. The ¹H-NMR (500 MHz, DMSO-d₆) spectrum of Compound C-10 is shown in FIG. 8.

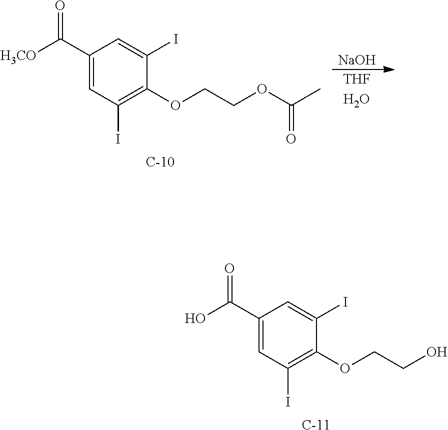

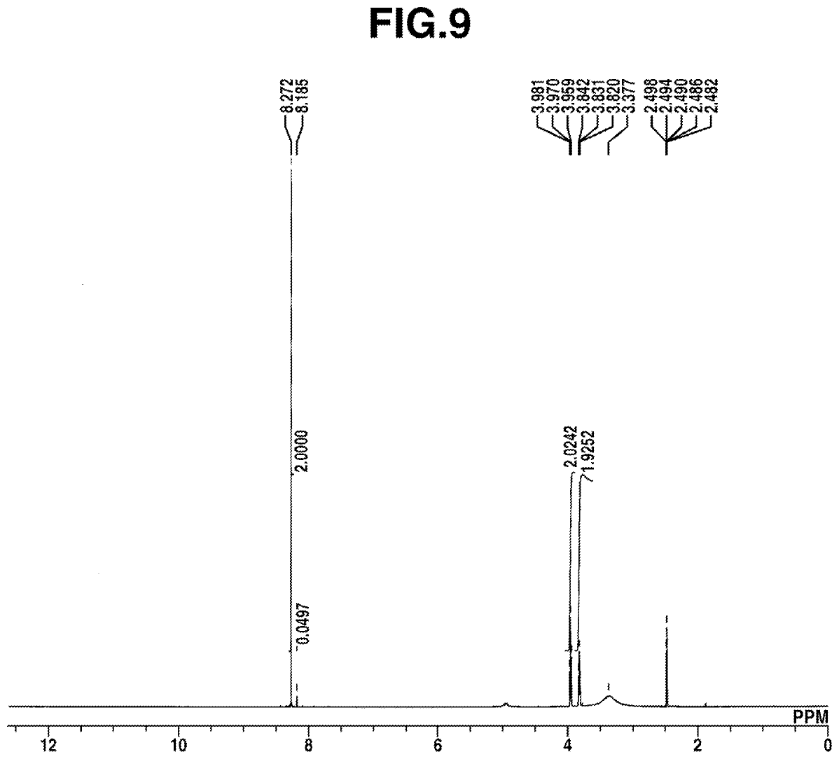

448 g of Compound C-10 was added to 1,200 g of THF, which was stirred under ice cooling. To the solution, 586 g of 25 wt % sodium hydroxide aqueous solution and 1,200 g of deionized water were added dropwise. Stirring was performed at 90° C. for 12 hours. At the end of reaction, the organic solvent was distilled off. 450 g of 20 wt % hydrochloric acid, 1,800 g of deionized water, and 1 L of hexane were added to the residue and stirred. Crystallization was followed by filtration, obtaining 353 g of Compound C-11 as solids. The ¹H-NMR (500 MHz, DMSO-d₆) spectrum of Compound C-11 is shown in FIG. 9.

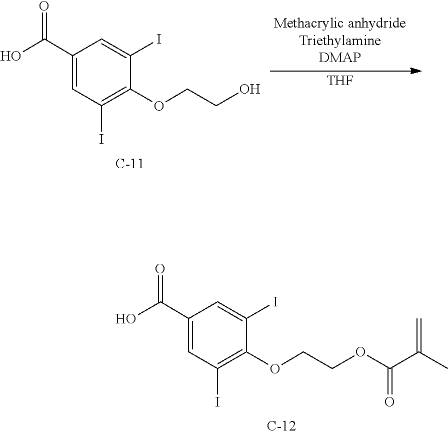

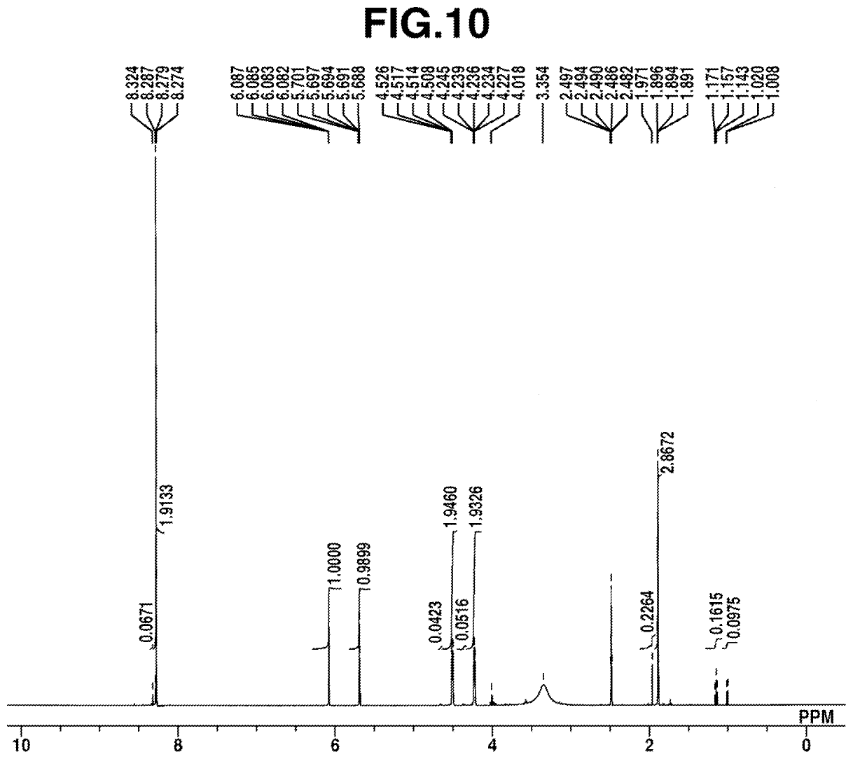

140 g of Compound C-11, 60 g of methacrylic anhydride, 640 g of THF, and an amount (1000 ppm/theoretical yield) of a polymerization inhibitor were mixed to form a solution. Under ice cooling, a mixture of 78 g of triethylamine, 3.9 g of DMAP and 200 g of THF was added dropwise to the solution. Stirring was performed for 3 hours under ice cooling. At the end of reaction, 420 g of 5 wt % sodium bicarbonate aqueous solution was added to the solution wider ice cooling, followed by 20 minutes of stirring. 260 g of 20 wt % hydrochloric acid was added to the solution and stirred for 20 minutes. 1,600 g of ethyl acetate was added to the solution and stirred for 30 minutes. The organic layer was taken out, washed with saturated brine and deionized water, and treated with active carbon. After the solvent was distilled off, 1,000 g of diisopropyl ether was added to the residue. Crystallization was followed by filtration, obtaining 144 g of Compound C-12 as solids. The ¹H-NMR (500 MHz, DMSO-d₆) spectrum of Compound C-12 is shown in FIG. 10.

(2) Synthesis of Acid Chloride Compound C-13

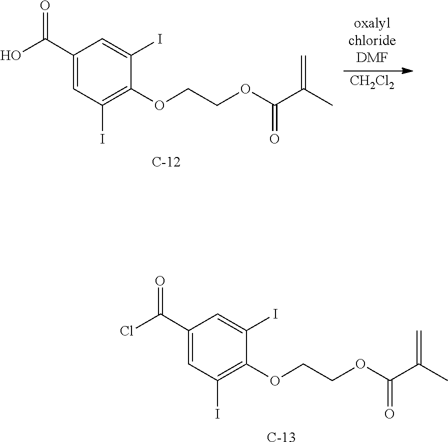

144 g of Compound C-12, 0.6 g of DMF, 1,200 g of methylene chloride, and an amount (1000 ppm/theoretical yield) of a polymerization inhibitor were mixed to form a solution. At room temperature, 44 g of oxalyl chloride was added dropwise to the solution. Stirring was performed at room temperature for 3 hours. At the end of stirring, the solvent was distilled off, obtaining 149 g of acid chloride compound C-13.

(3) Synthesis of Onium Salt Compound PAG-4

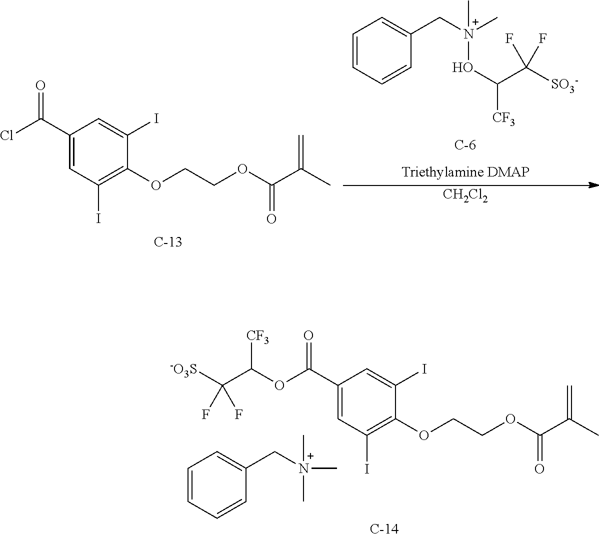

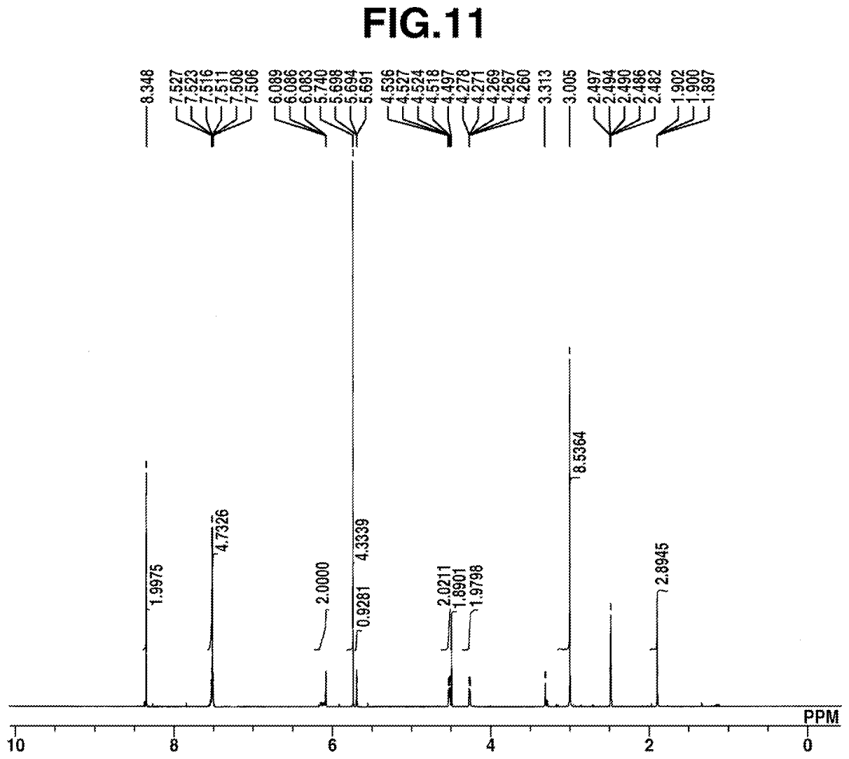

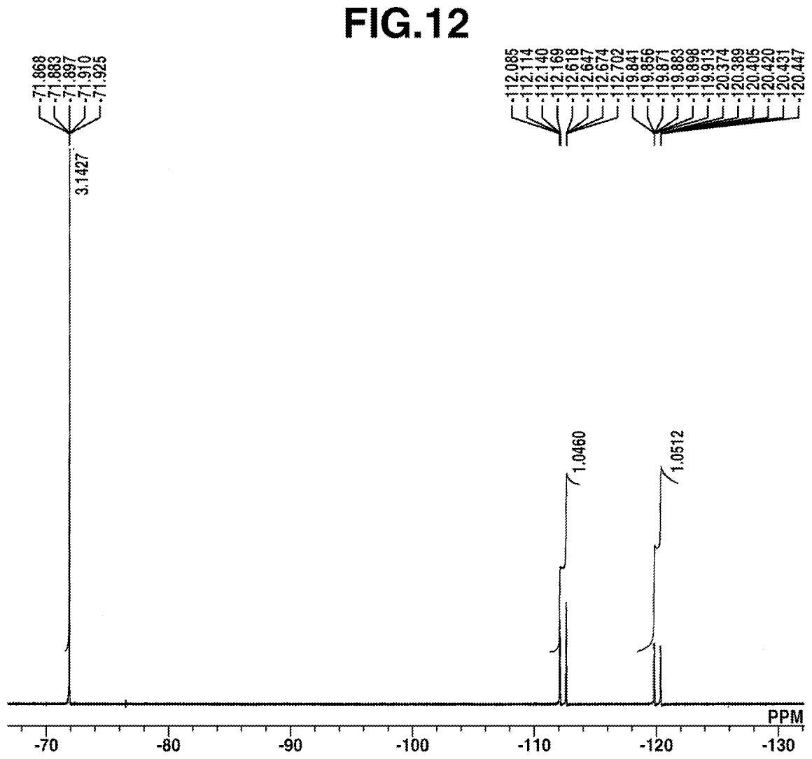

130 g of Compound C-6, 44 g of triethylamine, 3.5 g of DMAP, 600 g of methylene chloride, and an amount (1000 ppm/theoretical yield) of a polymerization inhibitor were mixed to form a solution. Under ice cooling, 149 g of Compound C-13 in 280 g of methylene chloride was added dropwise to the solution. Stirring was performed at room temperature for 20 hours. At the end of reaction, 250 g of 5 wt % hydrochloric acid aqueous solution was added under ice cooling, followed by 30 minutes of stirring. The organic layer was taken out and subjected to ordinary aqueous work-up. The solvent was distilled off, obtaining 205 g of Compound C-14 as oily matter. The ¹H-NMR (500 MHz, DMSO-d₆) spectrum of Compound C-14 is shown in FIG. 11. The ¹⁹F-NMR (500 MHz, DMSO-d₆) spectrum of Compound C-14 is shown in FIG. 12.

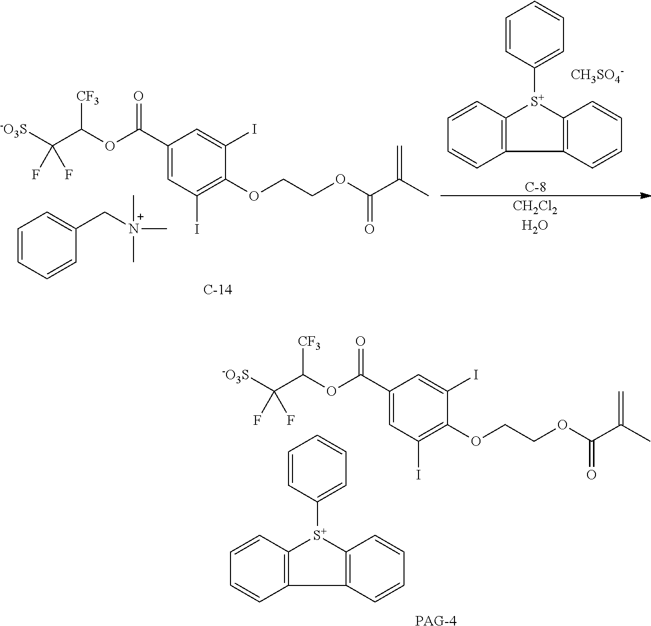

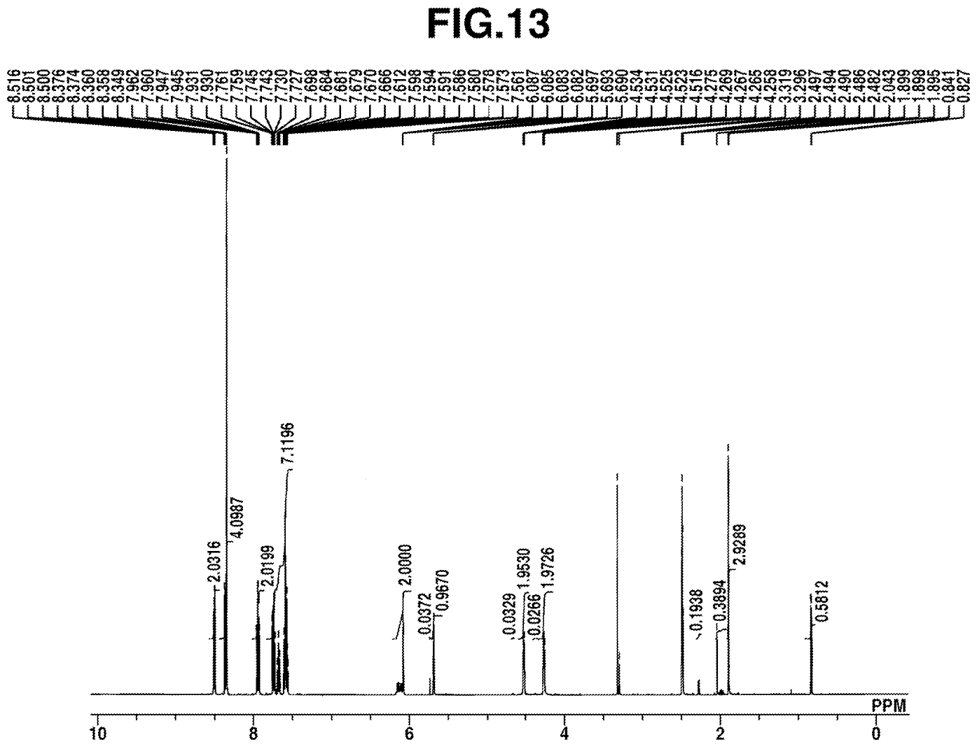

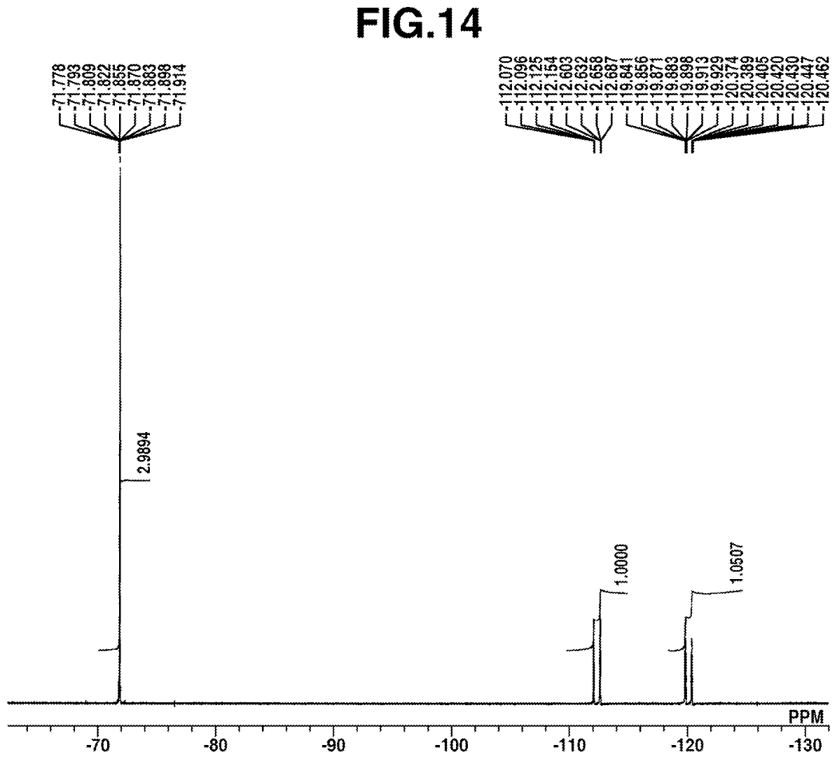

204 g of Compound C-14, 98 g of Compound C-8, 1,400 g of methylene chloride, 550 g of deionized water, and an amount (1000 ppm/theoretical yield) of a polymerization inhibitor were mixed and stirred at room temperature for 2 hours. The organic layer was taken out and subjected to ordinary aqueous work-up. After the solvent was distilled off, 250 g of MIBK was added to the residue and azeotroped to form a solution having a concentration of about 50 wt %, which was stirred at room temperature for 2 hours. 800 g of TBME was added to the solution and stirred. Crystallization was followed by filtration, obtaining 168 g of onium salt compound PAG-4 as solids. The ¹H-NMR (500 MHz, DMSO-d) spectrum of onium salt compound PAG-4 is shown in FIG. 13. The ¹⁹F-NMR (500 MHz, DMSO-d₆) spectrum of onium salt compound PAG-4 is shown in FIG. 14.

### Example 1-3: Synthesis of Onium Salt Compound PAG-7

PAG-7 was synthesized according to the following scheme.

(1) Synthesis of Compound C-19

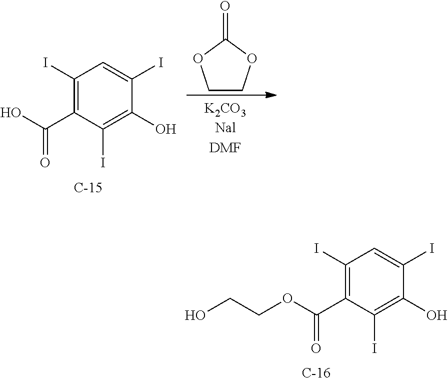

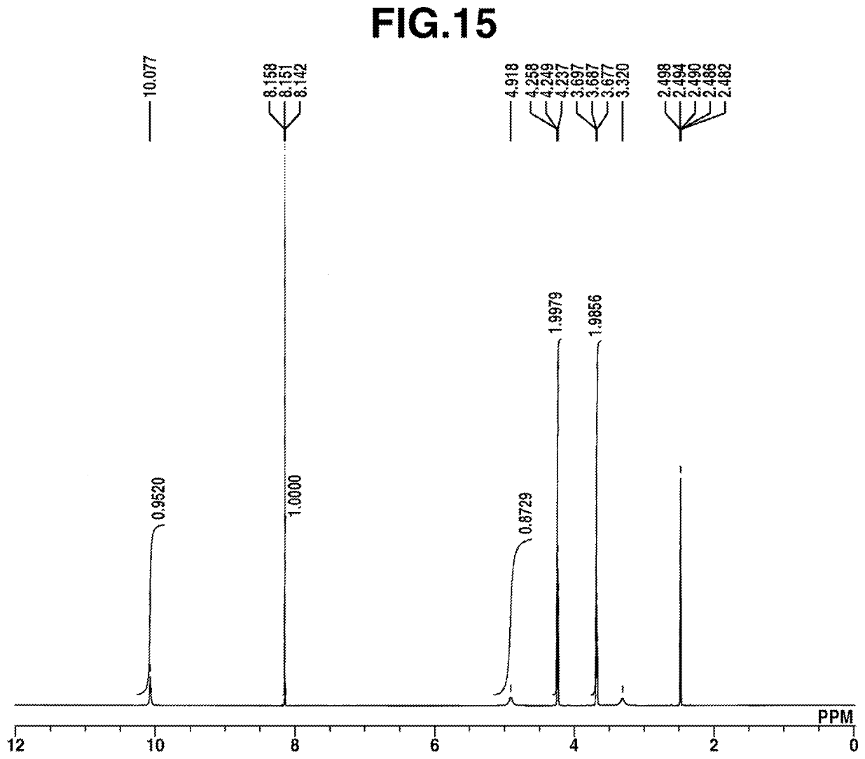

15 g of Reactant C-15, 6 g of ethylene carbonate, 12 g of potassium carbonate, and 105 g of DMF were mixed and stirred at 85° C. for 18 hours. At the end of stirring, the solution was ice cooled, and 83 g of 10 wt % hydrochloric acid was added to quench the reaction. 200 g of deionized water was added to the solution, which was stirred for 30 minutes. The precipitate was collected by filtration and dissolved in a mixture of 120 g of ethyl acetate and 30 g of THF. Thereafter, 50 g of deionized water was added and stirred for 10 minutes. The organic layer was taken out and subjected to ordinary aqueous work-up. After the solvent was distilled off, 35 g of hexane was added to the residue and stirred for 1 hour. Crystallization was followed by filtration. The solid was vacuum dried, obtaining 12 g of Compound C-16 as solids. The ¹H-NMR (500 MHz, DMSO-ds) spectrum of Compound C-16 is shown in FIG. 15.

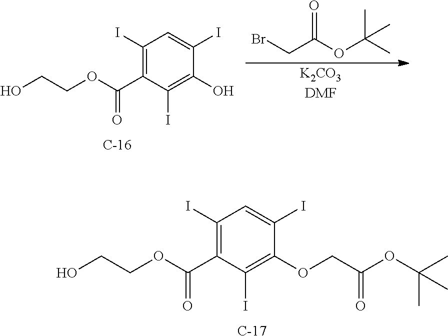

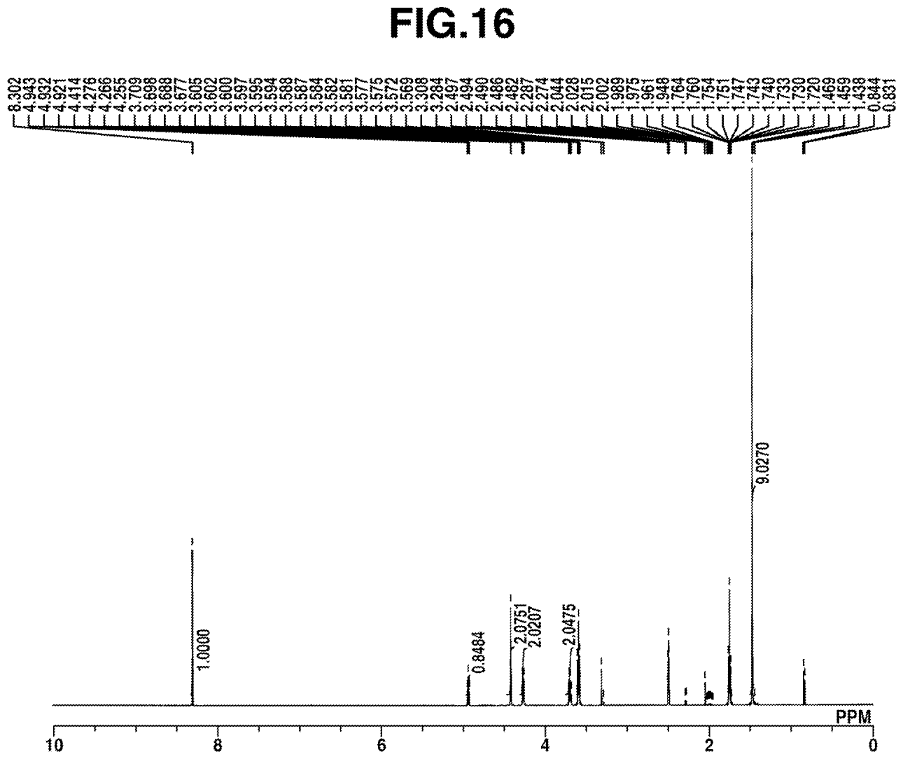

12 g of Compound C-16, 5.4 g of tert-butyl bromoacetate, 3.5 g of potassium carbonate, and 60 g of DMF were mixed and stirred at 30° C. for 3 hours. After stirring and subsequent ice cooling, 120 g of deionized water was added to the solution to quench the reaction. 120 g of MIBK was added to the solution, which was stirred for 10 minutes. The organic layer was taken out and subjected to ordinary aqueous work-up. After the solvent was distilled off, 40 g of hexane was added to the residue and stirred for 1 hour. With the supernatant removed, the remaining was dissolved in THF. The solvent was distilled off under reduced pressure, obtaining 15 g of Compound C-17 as red oily matter. The ¹H-NMR (500 MHz, DMSO-d₆) spectrum of Compound C-17 is shown in FIG. 16.

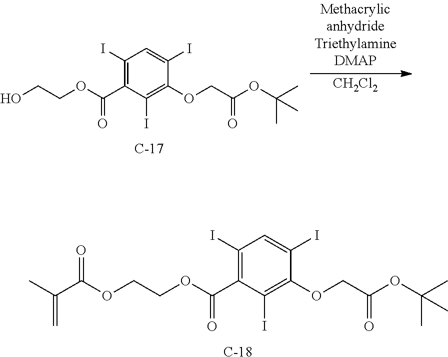

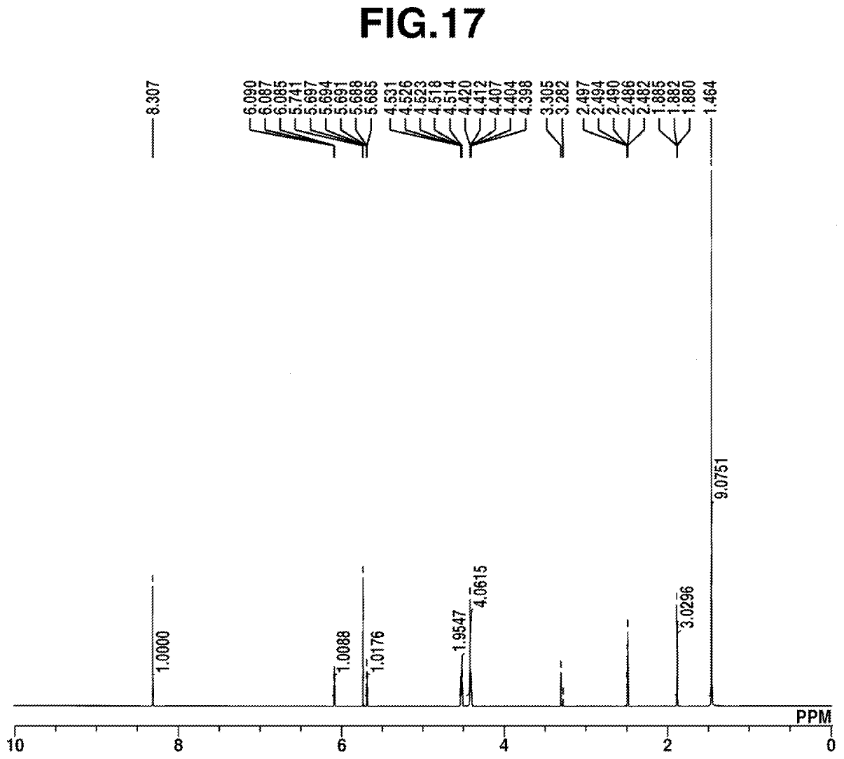

15 g of Compound C-17, 3.3 g of methacrylic anhydride, 60 g of methylene chloride, and a polymerization inhibitor were mixed. To the solution kept under ice cooling, a mixture of 2.5 g of triethylamine, 0.2 g of DMAP, and 5 g of methylene chloride was added dropwise, followed by 4 hours of stirring under ice cooling. At the end of reaction, 20 g of 5 wt % sodium bicarbonate aqueous solution was added under ice cooling, followed by 3 hours of stirring. The organic layer was taken out and subjected to ordinary aqueous work-up. The solvent was distilled off, obtaining 15 g of Compound C-18 as red oily matter. The ¹H-NMR (500 MHz, DMSO-d₆) spectrum of Compound C-18 is shown in FIG. 17.

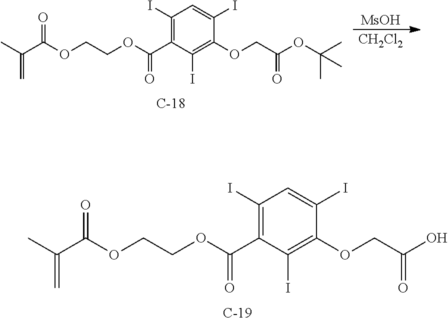

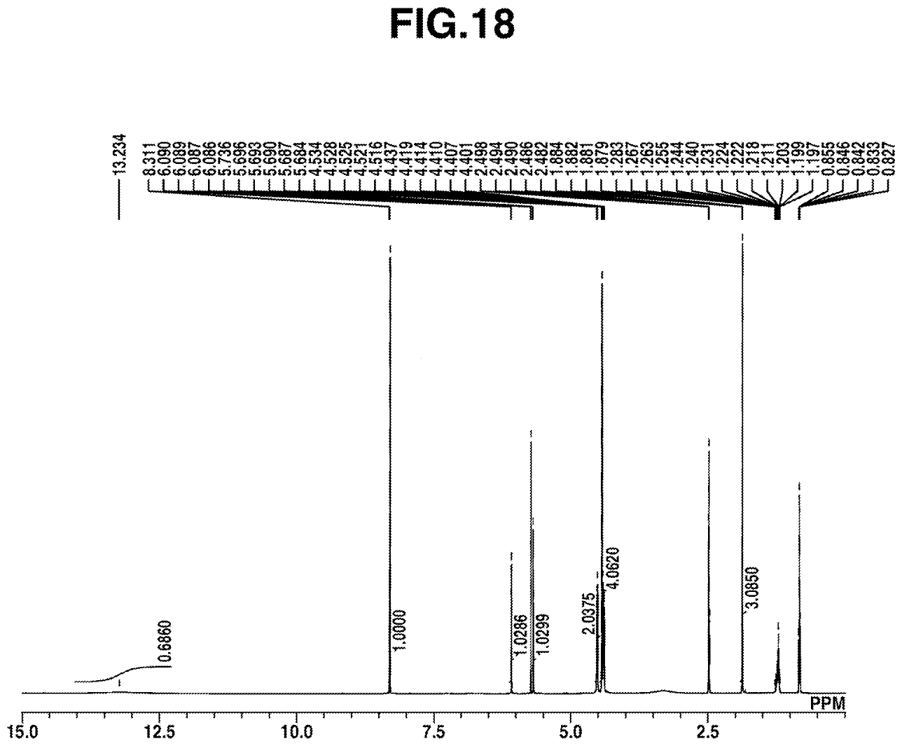

15 g of Compound C-18, 1.8 g of methanesulfonic acid, 60 g of methylene chloride, and a polymerization inhibitor were mixed and stirred at 40° C. for 7 hours. At the end of stirring, 16 g of 10 wt % sodium bicarbonate aqueous solution was added, followed by 20 minutes of stirring. The organic layer was taken out and subjected to ordinary aqueous work-up. After the solvent was distilled off, 40 g of hexane was added and stirred for 2 hours. With the supernatant removed, the remaining solvent was distilled off under reduced pressure, obtaining 13 g of Compound C-19 as red oily matter. The ¹H-NMR (500 MHz, DMSO-d₆) spectrum of Compound C-19 is shown in FIG. 18.

(2) Synthesis of Onium Salt Compound PAG-7

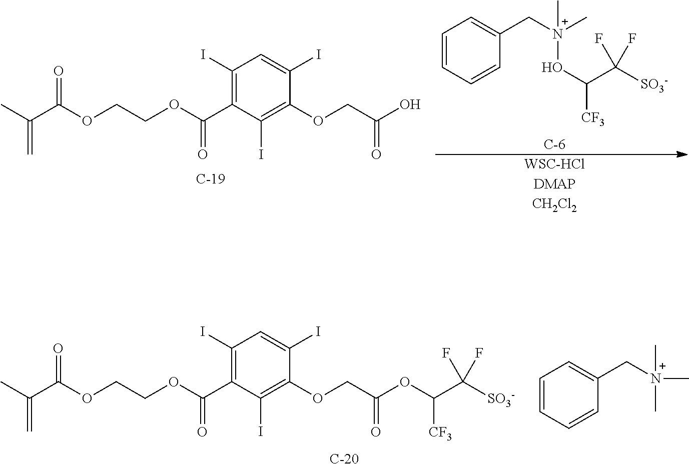

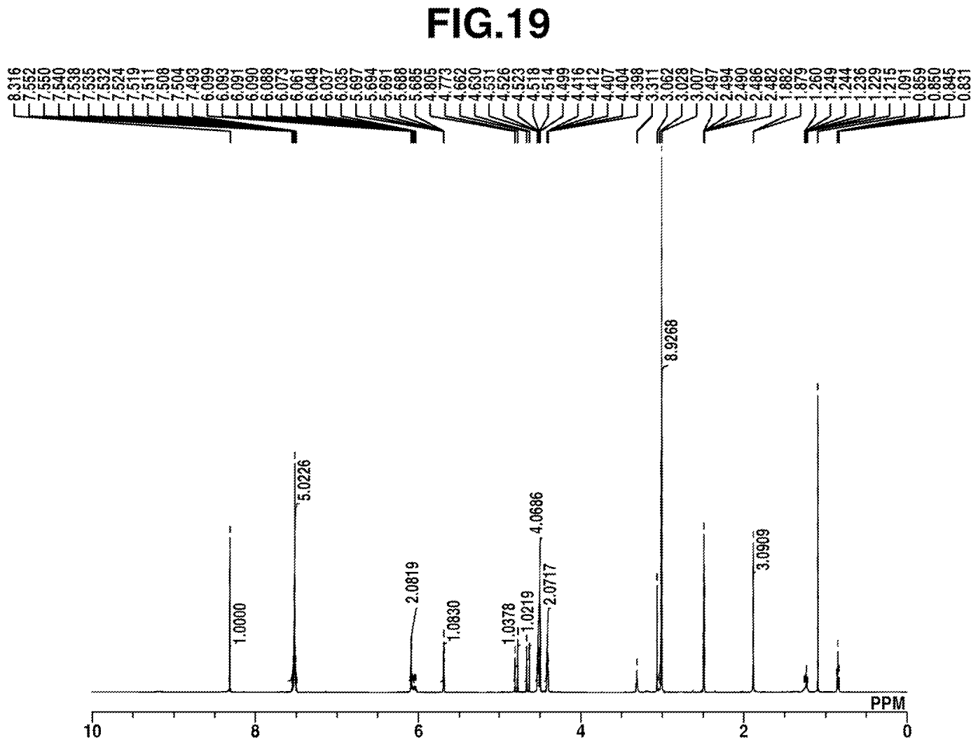

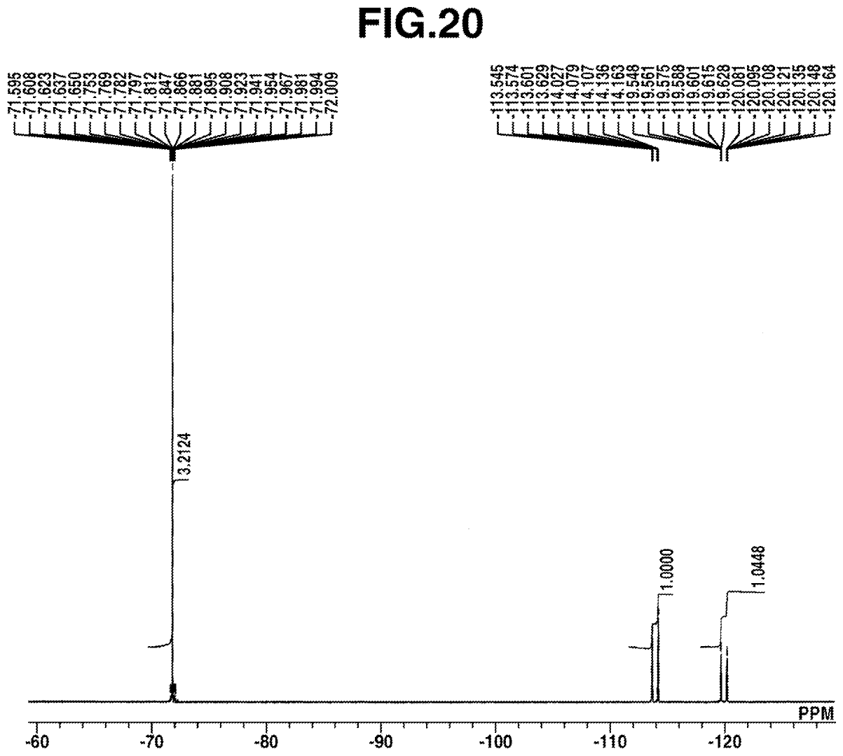

13 g of Compound C-19, 8.0 g of Compound C-6, 0.2 g of DMAP, 30 g of methylene chloride, and a polymerization inhibitor were mixed. To the solution kept at room temperature, 4.7 g of N-(3-dimethylaminopropyl)-N′-ethylcarbodiimide hydrochloride (WSC-HCl) was added, followed by 20 hours of stirring at room temperature. At the end of reaction, 20 g of deionized water was added to the reaction solution at room temperature, followed by 20 minutes of stirring. The organic layer was taken out and subjected to ordinary aqueous work-up. After the solvent was distilled off, 40 g of tert-butyl methyl ether was added to the residue and stirred. With the supernatant removed, 40 g of hexane was added to the remaining and stirred for 40 minutes. Crystallization was followed by filtration. The precipitate was dried under reduced pressure, obtaining 16 g of Compound C-20 as solids. The ¹H-NMR (500 MHz, DMSO-d₆) spectrum of Compound C-20 is shown in FIG. 19. The ¹⁹F-NMR (500 MHz, DMSO-d₆) spectrum of Compound C-20 is shown in FIG. 20.

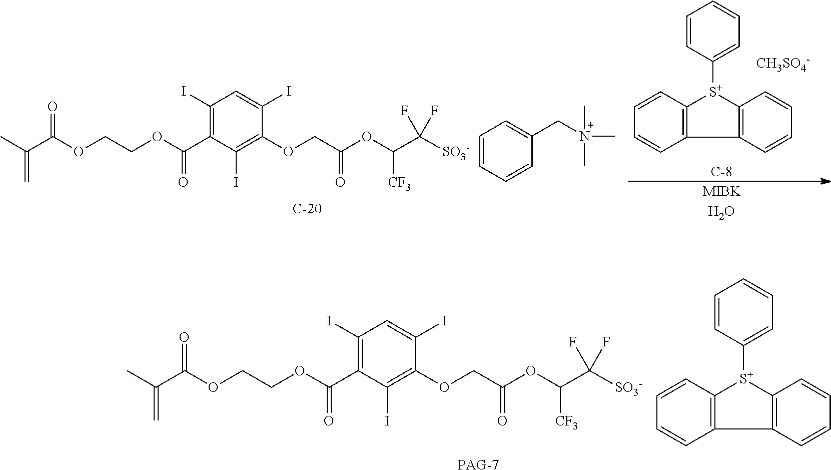

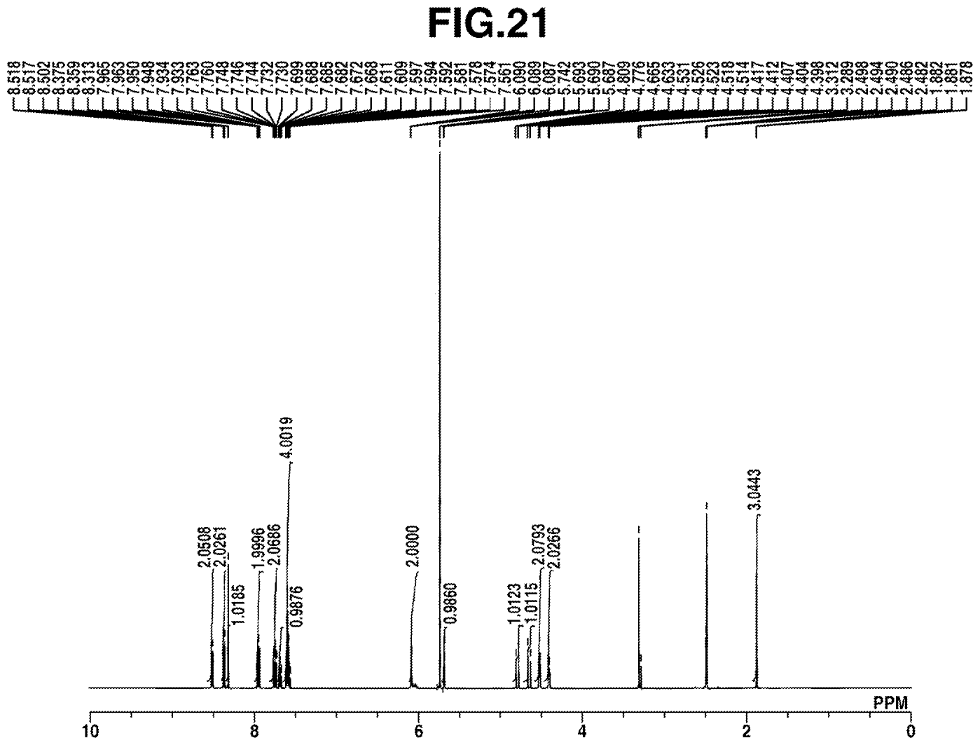

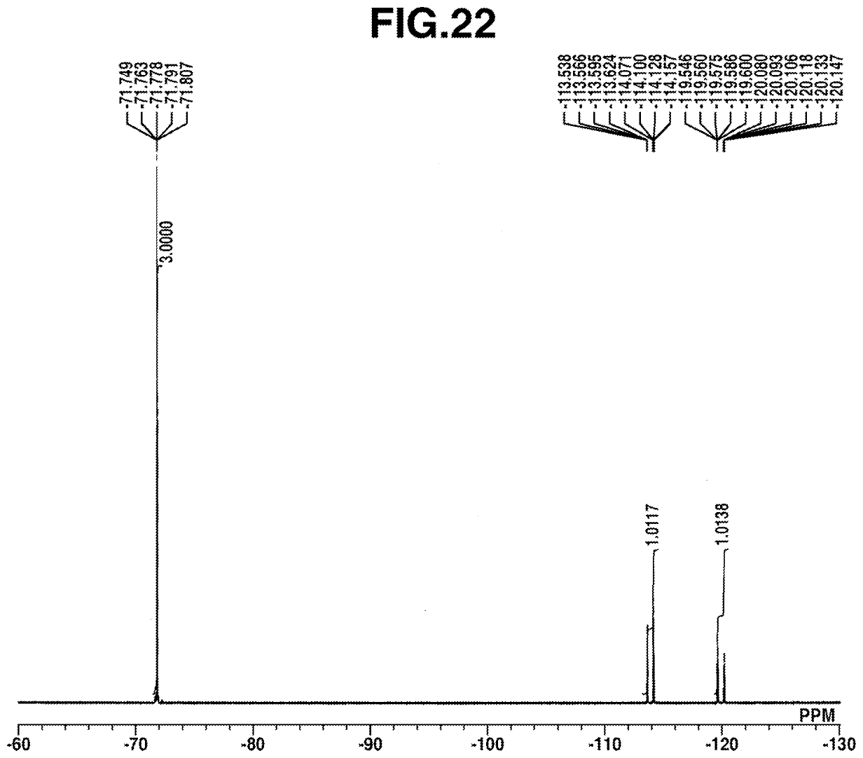

16 g of Compound C-20, 6.4 g of Compound C-8, 100 g of MIBK, 40 g of deionized water, and a polymerization inhibitor were mixed and stirred at room temperature for 2 hours. The organic layer was taken out and subjected to ordinary aqueous work-up. After the solvent was distilled off, the residue was purified by silica gel column chromatography, obtaining 13 g of onium salt compound PAG-7 as faintly yellow oily matter. The ¹H-NMR (500 MHz, DMSO-d₆) spectrum of onium salt compound PAG-7 is shown in FIG. 21. The ¹⁹F-NMR (500 MHz, DMSO-d₆) spectrum of onium salt compound PAG-7 is shown in FIG. 22.

### Examples 1-4 to 1-7: Synthesis of Onium Salt Compounds PAG-2, PAG-3, PAG-5, PAG-6 and PAG-8

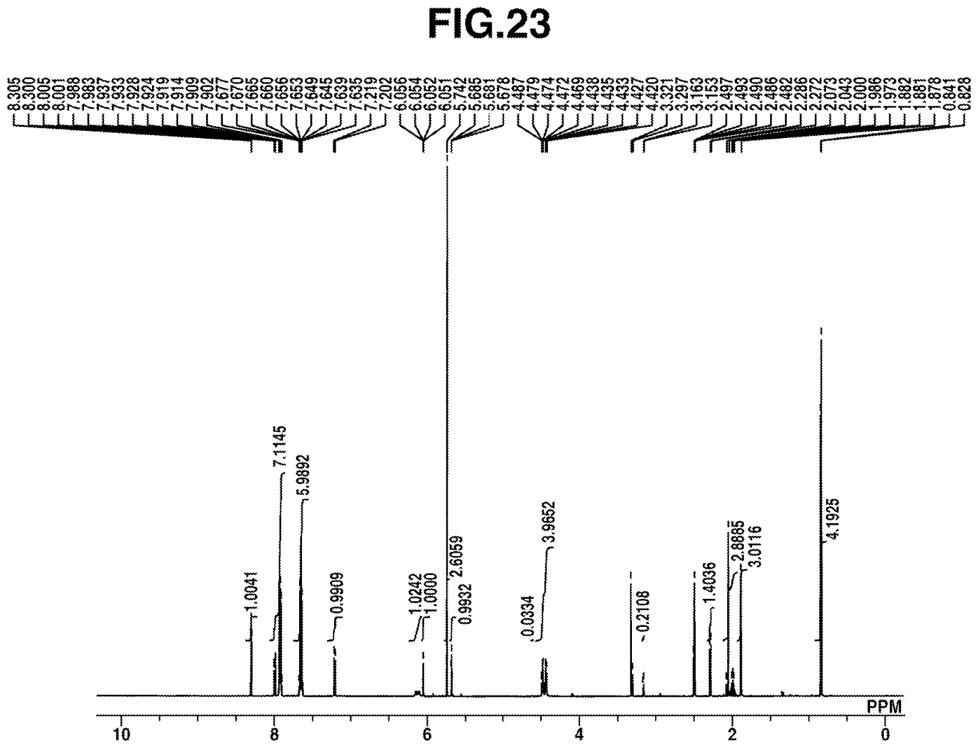

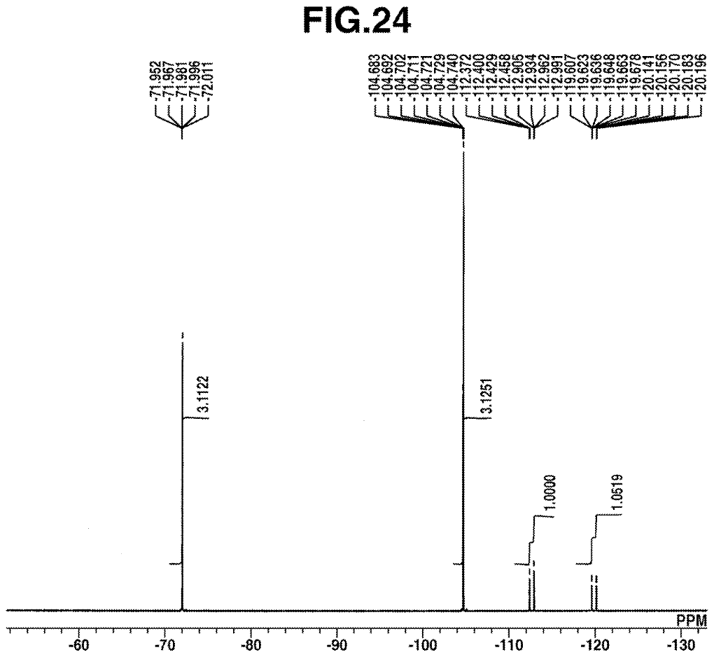

The following onium salt compounds PAG-2, PAG-3, PAG-5, PAG-6 and PAG-8 were synthesized by the same procedure as in Example 1-1 except that the reactant was changed. The ¹H-NMR (500 MHz, DMSO-d₆) spectrum of onium salt compound PAG-2 is shown in FIG. 23. The ¹⁹F-NMR (500 MHz, DMSO-d₆) spectrum of onium salt compound PAG-2 is shown in FIG. 24.

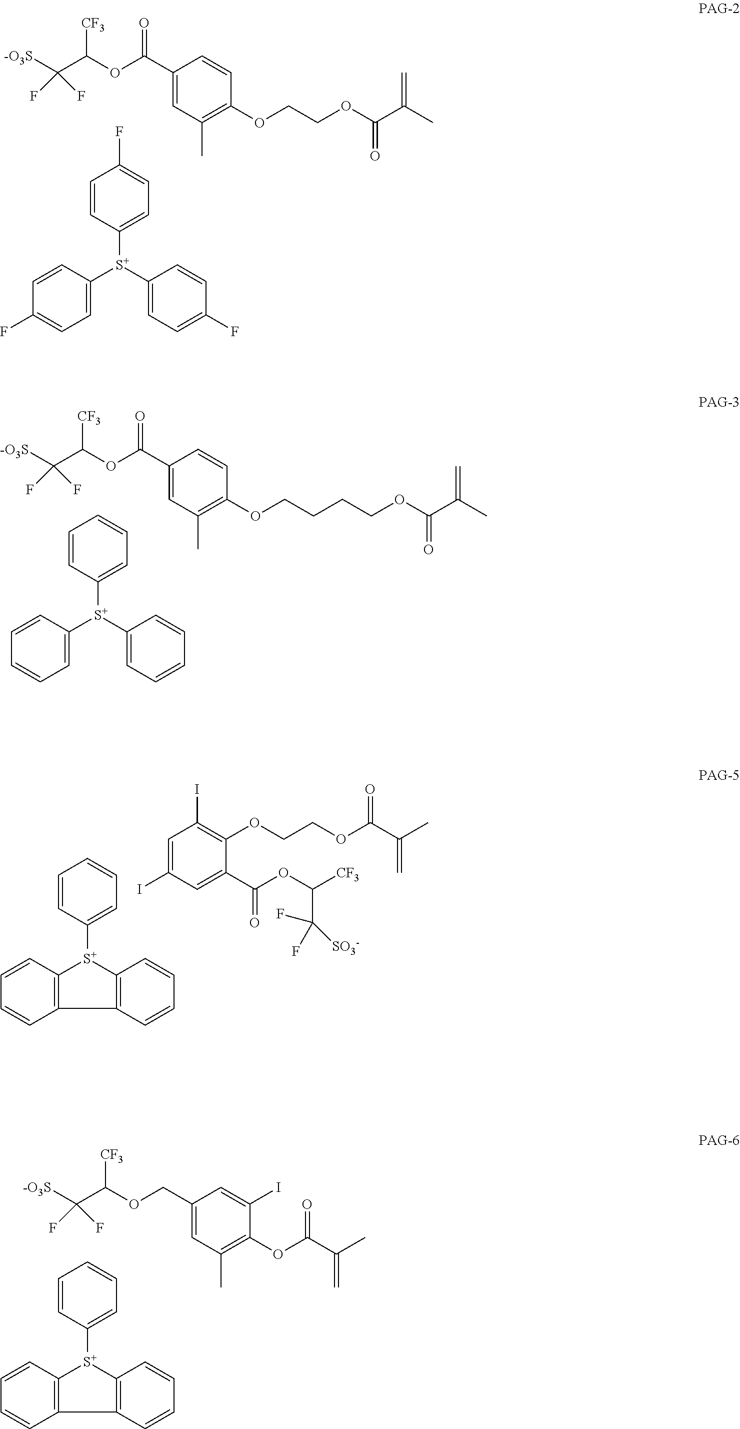

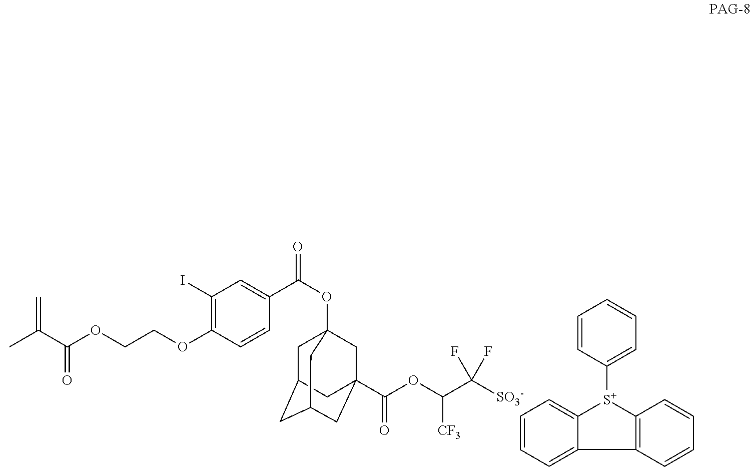

[2] Synthesis of Base Polymer

### Example 2-1: Synthesis of Polymer P-1

A flask under nitrogen atmosphere was charged with 25.1 g of PAG-1, 43.8 g of 1-tert-butylcyclopentyl methacrylate, 9.8 g of 3-hydroxyadamantyl-1-yl methacrylate, 21.3 g of oxotetrahydrofuran-3-yl methacrylate, 4.79 g of dimethyl 2,2′-azobis(isobutyrate), and 175 g of MEK to form a monomer solution. Another flask under nitrogen atmosphere was charged with 58 g of MEK, which was heated at 80° C. with stirring. The monomer solution was added dropwise to the MEK over 4 hours. At the end of addition, the polymerization solution was continuously stirred for 2 hours while maintaining the temperature at 80° C. After the solution was cooled to room temperature, it was added dropwise to a mixture of 100 g of MEK and 900 g of hexane. The solid precipitate was collected by filtration. The precipitate was washed twice with 600 g of hexane and vacuum dried at 50° C. for 20 hours, obtaining Polymer P-1 as white powder solids, having the composition shown in Table 1. Amount 91.2 g. yield 91%.

### Examples 2-2 to 2-18: Synthesis of Polymers P-2 to P-18

Polymers P-2 to P-12 and comparative Polymers P-13 to P-18, shown below, were synthesized by the same procedure as in Example 2-1 except that the type and amount (blending ratio) of monomers were changed.

The composition of Polymers P-1 to P-18 is shown in Table 1 wherein the incorporation ratio is a molar ratio.

TABLE 1

| | | Incorpo- | | Incorpo- | | Incorpo- | | Incorpo- | | |
|---|---|---|---|---|---|---|---|---|---|---|
| | | ration | | ration | | ration | | ration | | |
| Polymer | Unit 1 | ratio | Unit 2 | ratio | Unit 3 | ratio | Unit 4 | ratio | Mw | Mw/Ma |
| P-1 | PAG-1 | 0.12 | A-1 | 0.50 | B-2 | 0.38 | — | — | 10,900 | 1.91 |
| P-2 | PAG-1 | 0.15 | A-2 | 0.50 | B-2 | 0.35 | — | — | 11,600 | 1.90 |
| P-3 | PAG-1 | 0.20 | A-1 | 0.60 | B-3 | 0.20 | — | — | 11,000 | 1.81 |
| P-4 | PAG-2 | 0.15 | A-1 | 0.55 | B-2 | 0.30 | — | — | 10,100 | 1.78 |
| P-5 | PAG-2 | 0.20 | A-4 | 0.60 | B-3 | 0.20 | — | — | 10,300 | 1.92 |
| P-6 | PAG-3 | 0.10 | A-3 | 0.60 | B-1 | 0.30 | — | — | 10,000 | 1.82 |
| P-7 | PAG-4 | 0.15 | A-1 | 0.60 | B-2 | 0.25 | — | — | 9,900 | 1.75 |
| P-8 | PAG-5 | 0.20 | A-5 | 0.20 | B-2 | 0.60 | — | — | 10,200 | 1.89 |
| P-9 | PAG-6 | 0.15 | A-2 | 0.50 | B-2 | 0.35 | — | — | 10,600 | 1.92 |
| P-10 | PAG-6 | 0.20 | A-1 | 0.60 | B-3 | 0.20 | — | — | 11,000 | 1.81 |
| P-11 | PAG-7 | 0.10 | A-3 | 0.60 | B-1 | 0.30 | — | — | 10,800 | 1.88 |
| P-12 | PAG-8 | 0.15 | A-1 | 0.50 | B-2 | 0.35 | — | — | 10,200 | 1.90 |
| P-13 | C-1 | 0.20 | A-1 | 0.50 | A-4 | 0.20 | B-2 | 0.10 | 8,900 | 1.61 |
| P-14 | C-2 | 0.20 | A-3 | 0.20 | B-1 | 0.60 | — | — | 8,600 | 1.56 |
| P-15 | C-3 | 0.20 | A-1 | 0.60 | B-2 | 0.20 | — | — | 10,300 | 1.80 |
| P-16 | C-4 | 0.20 | A-3 | 0.40 | A-4 | 0.20 | B-2 | 0.20 | 9,100 | 1.62 |
| P-17 | A-1 | 0.30 | A-3 | 0.20 | A-4 | 0.30 | B-3 | 0.20 | 8,100 | 1.59 |
| P-18 | A-1 | 0.40 | A-4 | 0.10 | B-2 | 0.50 | — | — | 8,500 | 1.63 |

The structure of each unit in Table 1 is shown below.

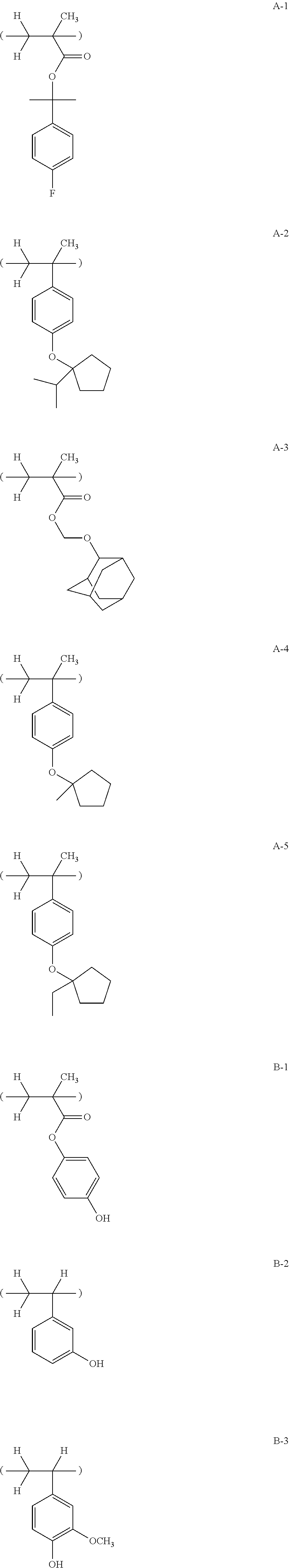

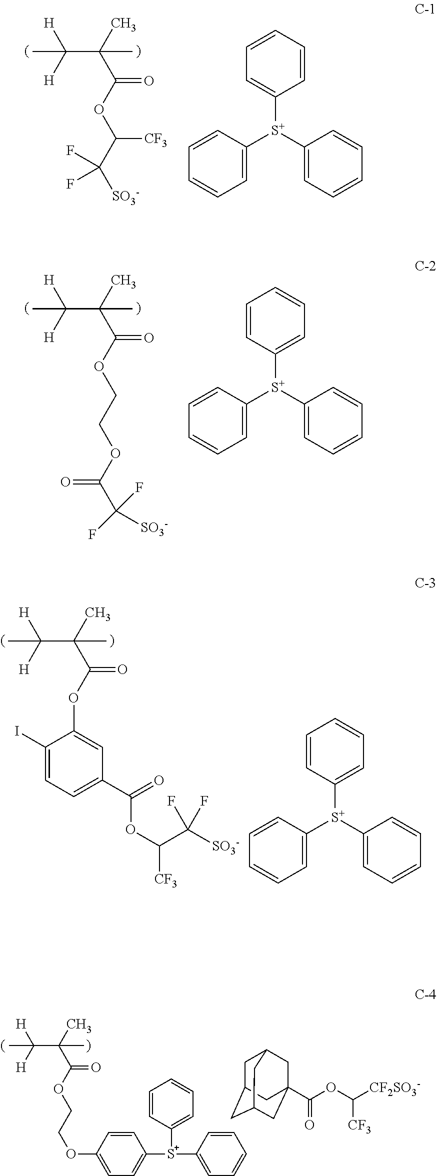

[3] Preparation of Resist Composition

### Examples 3-1 to 3-16 and Comparative Examples 3-1 to 3-7

A resist composition (R-01 to R-23) was prepared by dissolving a base polymer (Polymer P-1 to P-18), photoacid generator (PAG-A. PAG-B), quencher (AQ-1, SQ-1), and alkali-soluble surfactant (F-1) in a solvent containing 0.01% by weight of surfactant A in accordance with the formulation shown in Table 2, and filtering the solution through a Teflon® filter with a pore size of 0.2 μm.

The quencher (AQ-1, SQ-1), organic solvent, photoacid generator (PAG-A, PAG-B), and alkali-soluble surfactant (F-1) in Table 2 are identified below.

Organic Solvent
PGMEA: propylene glycol monomethyl ether acetate
CyHO: cyclohexanone
GBL: γ-butyrolactone

Quencher AQ-1: 2-(4-morpholinyl)ethyl octadecanoate

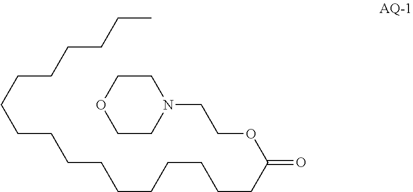 [SMILES: CCCCCCCCCCCCCCCCCC(=O)OCCN1CCOCC1]

Quencher SQ-1

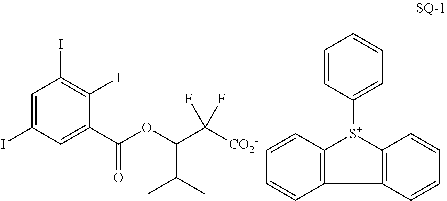

Photoacid Generator PAG-A

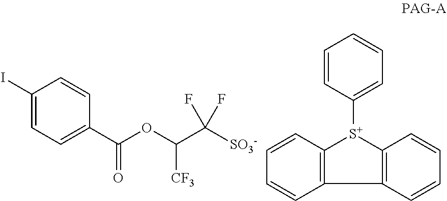

Photoacid Generator PAG-B

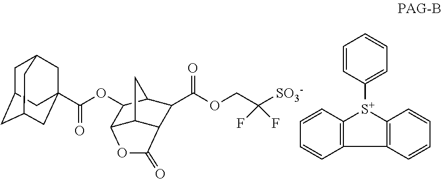

Photoacid Generator PAG-C

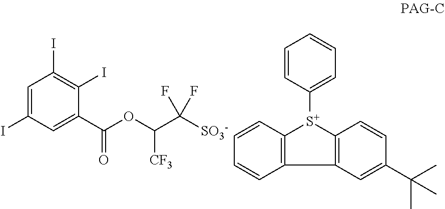

Alkali-soluble surfactant (F-1): poly(2,2,3,3,4,4,4-heptafluoro-1-isobutyl-1-butyl methacrylate/9-(2,2,2-trifluoro-1-trifluoromethylethyloxycarbonyl)-4-oxatricyclo[4.2.1.0^{3,7}]nonan-5-on-2-yl methacrylate)

Mw=7,700

Mw/Mn=1.82

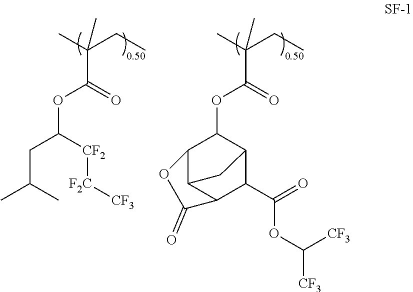

Surfactant A: 3-methyl-3-(2,2,2-trifluoroethoxymethyl)oxetane/tetrahydrofuran/2,2-dimethyl-1,3-propanediol copolymer (Omnova Solutions, Inc.)

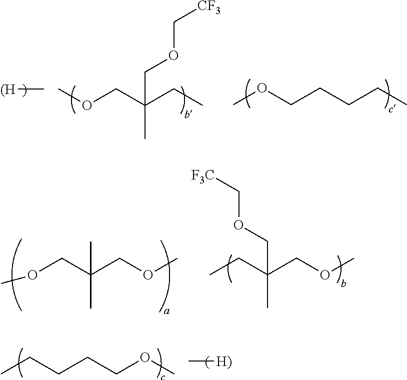

a:(b+b′):(c+c′)=1:4-7:0.01-1 (molar ratio)

Mw=1,500

TABLE 2

| | | | | Photoacid | | | | |
|---|---|---|---|---|---|---|---|---|
| | | Resist | Polymer | generator | Quencher | Surfactant | Solvent 1 | Solvent 2 |
| | | composition | (pbw) | (pbw) | (pbw) | (pbw) | (pbw) | (pbw) |
| Example | 3-1 | R-01 | P-1 | — | AQ-1 | — | PGMEA | CyHO |
| | | | (80) | | (0.6) | | (576) | (1728) |
| | 3-2 | R-02 | P-2 | — | AQ-1 | — | PGMEA | CyHO |
| | | | (80) | | (0.6) | | (576) | (1728) |
| | 3-3 | R-03 | P-3 | — | AQ-1 | — | PGMEA | CyHO |
| | | | (80) | | (0.6) | | (576) | (1728) |
| | 3-4 | R-04 | P-4 | — | AQ-1 | — | PGMEA | CyHO |
| | | | (80) | | (0.6) | | (576) | (1728) |
| | 3-5 | R-05 | P-5 | — | AQ-1 | — | PGMEA | CyHO |
| | | | (80) | | (0.6) | | (576) | (1728) |
| | 3-6 | R-06 | P-6 | — | AQ-1 | F-1 | PGMEA | CyHO |
| | | | (80) | | (0.6) | (5.0) | (576) | (1728) |
| | 3-7 | R-07 | P-7 | — | AQ-1 | F-1 | PGMEA | GBL |
| | | | (80) | | (0.6) | (5.0) | (1728) | (192) |
| | 3-8 | R-08 | P-8 | — | AQ-1 | F-1 | PGMEA | GBL |
| | | | (80) | | (0.6) | (5.0) | (1728) | (192) |
| | 3-9 | R-09 | P-9 | — | AQ-1 | F-1 | PGMEA | GBL |
| | | | (80) | | (0.6) | (5.0) | (1728) | (192) |
| | 3-10 | R-10 | P-10 | — | AQ-1 | F-1 | PGMEA | GBL |
| | | | (80) | | (0.6) | (5.0) | (1728) | (192) |
| | 3-11 | R-11 | P-11 | — | AQ-1 | F-1 | PGMEA | GBL |
| | | | (80) | | (0.6) | (5.0) | (1728) | (192) |
| | 3-12 | R-12 | P-12 | — | AQ-1 | F-1 | PGMEA | GBL |
| | | | (80) | | (0.6) | (5.0) | (1728) | (192) |
| | 3-13 | R-13 | P-1 | PAG-A (4.0) | SQ-1 | F-1 | PGMEA | CyHO |
| | | | (80) | | (0.6) | (5.0) | (576) | (1728) |
| | 3-14 | R-14 | P-1 | PAG-B (4.0) | SQ-1 | — | PGMEA | CyHO |
| | | | (80) | | (0.6) | | (576) | (1728) |
| | 3-15 | R-15 | P-2 | PAG-A (2.0) | SQ-1 | F-1 | PGMEA | CyHO |
| | | | (80) | PAG-B (1.0) | (0.6) | (5.0) | (1728) | (192) |
| | 3-16 | R-16 | P-9 | PAG-A (2.0) | SQ-1 | F-1 | PGMEA | CyHO |
| | | | (80) | PAG-B (1.0) | (0.6) | (5.0) | (1728) | (192) |
| Comparative | 3-1 | R-17 | P-13 | — | AQ-1 | — | PGMEA | CyHO |
| Example | | | (80) | | (0.6) | | (576) | (1728) |
| | 3-2 | R-18 | P-14 | — | AQ-1 | — | PGMEA | CyHO |
| | | | (80) | | (0.6) | | (576) | (1728) |
| | 3-3 | R-19 | P-15 | — | AQ-1 | F-1 | PGMEA | CyHO |
| | | | (80) | | (0.6) | (5.0) | (576) | (1728) |
| | 3-4 | R-20 | P-16 | — | AQ-1 | — | PGMEA | GBL |
| | | | (80) | | (0.6) | | (1728) | (192) |
| | 3-5 | R-21 | P-17 | PAG-A | AQ-1 | — | PGMEA | GBL |
| | | | (80) | (27) | (0.6) | | (1728) | (192) |
| | 3-6 | R-22 | P-17 | PAG-C | AQ-1 | — | PGMEA | CyHO |
| | | | (80) | (21) | (0.6) | | (576) | (1728) |
| | 3-7 | R-23 | P-18 | PAG-B | AQ-1 | F-1 | PGMEA | CyHO |
| | | | (80) | (13) | (0.6) | (5.0) | (576) | (1728) |

[4] EUV Lithography Test 1

### Examples 4-1 to 4-16 and Comparative Examples 4-1 to 4-7

Each of the resist compositions (R-01 to R-23) was spin coated on a silicon substrate having a 20-mu coating of silicon-containing spin-on hard mask SHB-A940 (Shin-Etsu Chemical Co., Ltd., silicon content 43 wt %) and prebaked on a hotplate at 100° C. for 60 seconds to form a resist film of 40 nm thick. Using an EUV scanner NXE3400 (ASML, NA 0.33, a 0.9, 90° dipole illumination), the resist film was exposed to EUV through a mask bearing a 22-nm 1:1 line-and-space (LS) pattern. The resist film was baked (PEB) on a hotplate at 90° C. for 60 seconds and developed in a 2.38 wt % TMAH aqueous solution for 30 seconds to form a LS pattern.

The LS pattern was observed under CD-SEM (CG-5000, Hitachi High-Technologies Corp.) and evaluated for sensitivity, MEF and LWR by the following methods. The results are shown in Table 3.

Evaluation of Sensitivity

The optimum dose (Eop, mJ/cm²) which provided an LS pattern with a space width of 26 am and a pitch of 52 nm was determined and reported as sensitivity.

Evaluation of Mask Error Factor (MEF)

An LS pattern was formed by exposure in the optimum dose (Eop) with the mask pitch fixed and the mask space width varied. MEF was calculated from variations of the mask space width and the pattern space width according to the following equation:

MEF=(pattern space width)/(mask space width)−b

wherein b is a constant. A value closer to unity (1) indicates better performance.

Evaluation of LWR

An LS pattern was formed by exposure in the optimum dose (Eop). The space width was measured at longitudinally spaced apart 10 points, from which a 3-fold value (3σ) of the standard deviation (σ) was determined and reported as LWR. A smaller value indicates a pattern having a lower roughness and more uniform space width.

TABLE 3

| | | Resist | Eop | | |
|---|---|---|---|---|---|
| | | composition | (mJ/cm²) | MEF | LWR (nm) |
| Example | 4-1 | R-01 | 28 | 2.2 | 2.8 |
| | 4-2 | R-02 | 26 | 2.6 | 2.5 |
| | 4-3 | R-03 | 25 | 2.2 | 2.4 |
| | 4-4 | R-04 | 27 | 2.5 | 2.8 |
| | 4-5 | R-05 | 28 | 2.5 | 2.8 |
| | 4-6 | R-06 | 28 | 2.4 | 2.6 |
| | 4-7 | R-07 | 27 | 2.3 | 2.6 |
| | 4-8 | R-08 | 26 | 2.6 | 2.4 |
| | 4-9 | R-09 | 24 | 2.1 | 2.3 |
| | 4-10 | R-10 | 25 | 2.4 | 2.2 |
| | 4-11 | R-11 | 23 | 2.3 | 2.4 |
| | 4-12 | R-12 | 24 | 2.6 | 2.7 |
| | 4-13 | R-13 | 24 | 2.2 | 2.2 |
| | 4-14 | R-14 | 27 | 2.4 | 2.8 |
| | 4-15 | R-15 | 26 | 2.4 | 2.3 |
| | 4-16 | R-16 | 25 | 2.3 | 2.5 |
| Comparative | 4-1 | R-17 | 37 | 4.0 | 4.6 |
| Example | 4-2 | R-18 | 36 | 3.8 | 4.4 |
| | 4-3 | R-19 | 30 | 4.1 | 3.6 |
| | 4-4 | R-20 | 37 | 3.8 | 4.5 |
| | 4-5 | R-21 | 36 | 4.3 | 4.2 |
| | 4-6 | R-22 | 37 | 3.6 | 3.9 |
| | 4-7 | R-23 | 37 | 4.0 | 4.0 |

[5] EUV Lithography Test 2

### Examples 5-1 to 5-16 and Comparative Examples 5-1 to 5-7

Each of the resist compositions (R-01 to R-23) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, σ0.9, quadrupole illumination), the resist film was exposed to EUV through a mask bearing a hole pattern with a pitch 40 mu (on-wafer size) and +20% bias. The resist film was baked (PEB) on a hotplate at 85° C. for 60 seconds and developed in a 2.38 wt % TMAH aqueous solution for 30 seconds to form a hole pattern.

The hole pattern was observed under CD-SEM (CG-6300, Hitachi High-Technologies Corp.) and evaluated for sensitivity, MEF and CDU by the following methods. The results are shown in Table 4.

Evaluation of Sensitivity

The optimum dose (Eop, mJ/cm²) which provided a hole pattern with a size of 40 nm was determined and reported as sensitivity.

Evaluation of MEF

Exposure was made through a mask having a fixed pitch and a varying dot size, scaled as on-wafer size at the optimum dose (Eop). The size of the hole pattern transferred to the wafer was measured. With respect to the hole size, the size of the transferred pattern is plotted relative to the mask design size, and a gradient is computed by linear approximation, and reported as MEF. A smaller value of MEF indicates reduced influence of a finish error of the mask pattern and is better.

Evaluation of CDU

The hole pattern printed at the optimum dose (Eop) was observed. The size of 50 holes was measured, from which a 3-fold value (3σ) of the standard deviation (σ) was computed and reported as CDU. A smaller value indicates a hole pattern having better CDU.

TABLE 4

| | | Resist | Eop | | |
|---|---|---|---|---|---|
| | | composition | (mJ/cm²) | MEF | CDU (nm) |
| Example | 5-1 | R-01 | 27 | 2.4 | 3.0 |
| | 5-2 | R-02 | 26 | 2.8 | 2.8 |
| | 5-3 | R-03 | 25 | 2.2 | 2.4 |
| | 5-4 | R-04 | 26 | 2.4 | 3.0 |
| | 5-5 | R-05 | 28 | 2.5 | 2.8 |
| | 5-6 | R-06 | 28 | 3.0 | 2.6 |
| | 5-7 | R-07 | 26 | 2.3 | 2.4 |
| | 5-8 | R-08 | 26 | 2.6 | 2.7 |
| | 5-9 | R-09 | 23 | 2.2 | 2.4 |
| | 5-10 | R-10 | 24 | 2.4 | 2.6 |
| | 5-11 | R-11 | 22 | 2.3 | 2.2 |
| | 5-12 | R-12 | 25 | 2.6 | 2.5 |
| | 5-13 | R-13 | 24 | 2.2 | 2.6 |
| | 5-14 | R-14 | 27 | 2.4 | 2.7 |
| | 5-15 | R-15 | 26 | 2.4 | 2.4 |
| | 5-16 | R-16 | 23 | 2.4 | 2.5 |
| Comparative | 5-1 | R-17 | 37 | 4.1 | 4.6 |
| Example | 5-2 | R-18 | 36 | 4.3 | 4.2 |
| | 5-3 | R-19 | 29 | 3.0 | 3.8 |
| | 5-4 | R-20 | 37 | 3.6 | 4.6 |
| | 5-5 | R-21 | 36 | 4.0 | 4.2 |
| | 5-6 | R-22 | 37 | 3.9 | 4.0 |
| | 5-7 | R-23 | 37 | 4.3 | 4.2 |

It is evident from Tables 3 and 4 that resist compositions comprising a polymer comprising repeat units derived from an onium salt compound within the scope of the invention exhibit a high sensitivity and satisfactory values of MEF, LWR and CDU and are suited as the EUV lithography material.

Japanese Patent Application No. 2022-084705 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An onium salt compound consisting of a sulfonate anion having the structure that a polymerizable unsaturated bond is linked to an aromatic group substituted with at least one iodine atom via a carbon chain having at least two carbon atoms and a sulfonium or iodonium cation.

2. The onium salt compound of claim 1, having the formula (1): 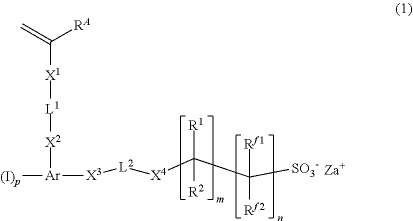
wherein m is an integer of 0 to 4, n is an integer of 1 to 4, p is an integer of 1 to 4,
R^{A} is hydrogen or methyl,
R¹ and R² are each independently hydrogen, fluorine, or a C₁-C₁₀ hydrocarbyl group which may contain a heteroatom, R¹ and R² may bond together to form a ring with the carbon atom to which they are attached,
R^{f1} and R^{f2} are each independently hydrogen, fluorine or trifluoromethyl, at least one of R^{f1} and R^{f2} is fluorine or trifluoromethyl,
X¹ to X⁴ are a single bond, ether bond, ester bond, sulfonic ester bond or carbonate bond,
L¹ is a C₂-C₁₅ hydrocarbylene group in which some or all hydrogen may be substituted by a heteroatom-containing moiety, and some constituent —CH₂— may be replaced by an ether bond, ester bond or lactone ring-containing moiety,
L² is a single bond or a C₁-C₁₅ hydrocarbylene group in which some or all hydrogen may be substituted by a heteroatom-containing moiety, and some constituent —CH₂— may be replaced by an ether bond, ester bond or lactone ring-containing moiety,
Ar is a C₆-C₁₅ (p+2)-valent aromatic group in which some or all hydrogen may be substituted by a substituent, and
Za⁺ is a sulfonium or iodonium cation.

3. The onium salt compound of claim 2 wherein the anion has the formula (1a): 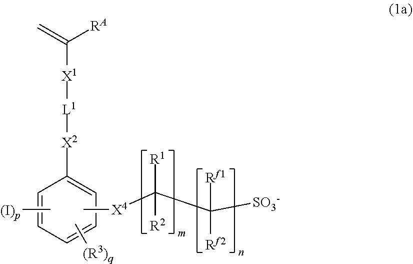
wherein m, n, p, R^{A}, R¹, R², R^{f1}, R^{f2}, X¹, X², X⁴ and L¹ are as defined above,
q is an integer of 0 to 3, q+p is from 1 to 4,
R³ is a hydroxy group, fluorine, amino group, sulfo group, or a C₁-C₁₅ hydrocarbyl group in which some or all hydrogen may be substituted by a heteroatom-containing moiety, and some constituent —CH₂— may be replaced by —O—, —C(═O)— or —N(R^{N})—, R^{N} is hydrogen or a C₁-C₁₀ hydrocarbyl group in which some or all hydrogen may be substituted by a heteroatom-containing moiety, and some constituent —CH₂— may be replaced by —O—, —C(═O)— or —S(═O)₂—.

4. The onium salt compound of claim 3 wherein the anion has the formula (1b): 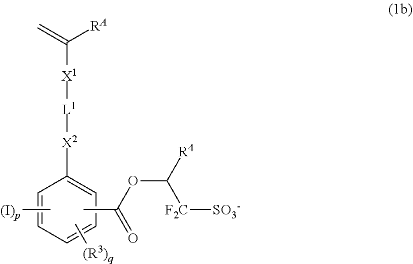
wherein p, q, R, R³, X¹, X², and L¹ are as defined above, and R⁴ is hydrogen or trifluoromethyl.

5. The onium salt compound of claim 1 wherein Za⁺ is a cation having the formula (Z-1) or (Z-2): 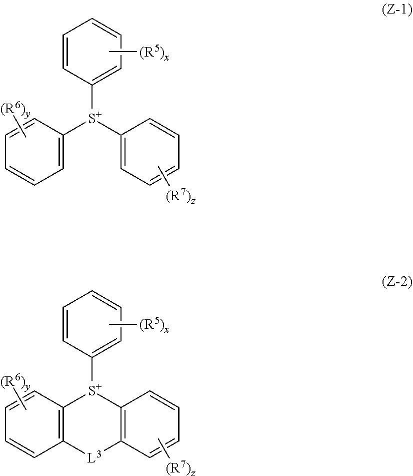
wherein R⁵, R⁶, and R⁷ are each independently halogen, hydroxy or a C₁-C₁₅ hydrocarbyl group in which some or all hydrogen may be substituted by a heteroatom-containing moiety, and some constituent —CH₂— may be replaced by —O—, —C(═O)—, —S—, —S(═O)—, —S(═O)₂— or —N(R^{N})—,
L³ is a single bond, —CH₂—, —O—, —C(═O)—, —S—, —S(═O)—, —S(═O)₂— or —N(R^{N})—,
R^{N} is hydrogen or a C₁-C₁₀ hydrocarbyl group in which some or all hydrogen may be substituted by a heteroatom-containing moiety, and some constituent —CH₂— may be replaced by —O—, —C(═O)— or —S(═O)₂—,
x, y and z are each independently an integer of 0 to 5, with the proviso that when x is 2 or more, a plurality of R⁵ may be identical or different, and two R⁵ may bond together to form a ring with the carbon atoms on the benzene ring to which they are attached, when y is 2 or more, a plurality of R⁶ may be identical or different, and two R⁶ may bond together to form a ring with the carbon atoms on the benzene ring to which they are attached, when z is 2 or more, a plurality of R⁷ may be identical or different, and two R⁷ may bond together to form a ring with the carbon atoms on the benzene ring to which they are attached.

6. A polymer comprising repeat units derived from the onium salt compound of claim 1.

7. A resist composition comprising a base polymer containing the polymer of claim 6 and an organic solvent.

8. The resist composition of claim 7 wherein the polymer further comprises repeat units having the formula (b1) or (b2): 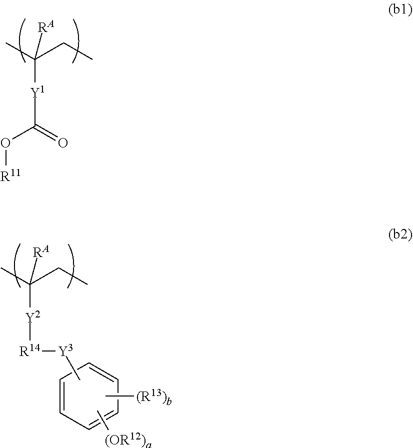
wherein R^{A} is as defined above,
Y¹ is a single bond, phenylene, naphthylene, or a C₁-C₁₂ linking group containing at least one moiety selected from ester bond and lactone ring,
Y² is a single bond or ester bond,
Y³ is a single bond, ether bond or ester bond,
R¹¹ and R¹² are each independently an acid labile group,
R¹³ is fluorine, trifluoromethyl, cyano or a C₁-C₆ saturated hydrocarbyl group,
R¹⁴ is a single bond or a C₁-C₆ alkanediyl group in which some —CH₂— may be replaced by an ether bond or ester bond, and
a is 1 or 2, b is an integer of 0 to 4, and a+b is from 1 to 5.

9. The resist composition of claim 7 wherein the polymer further comprises repeat units having the formula (c): 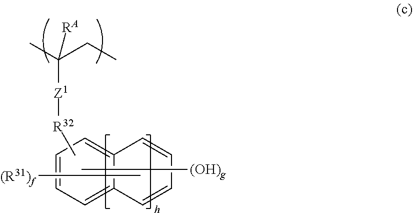
wherein R^{A} is as defined above,
Z¹ is a single bond, ether bond, ester bond, sulfonic ester bond, or carbonate bond,
R³¹ is fluorine, iodine or a C₁-C₁₀ hydrocarbyl group in which some —CH₂— may be replaced by —O— or —C(═O)—,
R³² is a single bond or a C₁-C₁₅ hydrocarbylene group,
f is an integer meeting 0≤f≤5+2h−g, g is an integer of 1 to 3, and h is an integer of 0 to 2.

10. The resist composition of claim 7, further comprising a quencher.

11. The resist composition of claim 7, further comprising a photoacid generator.

12. The resist composition of claim 7, further comprising a surfactant.

13. A pattern forming process comprising the steps of applying the resist composition of claim 7 onto a substrate to form a resist film thereon, exposing the resist film to high-energy radiation, and developing the exposed resist film in a developer.

14. The process of claim 13 wherein the high-energy radiation is ArF excimer laser of wavelength 193 nm, KrF excimer laser of wavelength 248 nm, EB, or EUV of wavelength 3 to 15 nm.
